(19) United States
(12) Patent Application Publication
(10) Pub. No.: US 2024/0301408 A1
(43) Pub. Date: Sep. 12, 2024

(54) MICRORNA 195 COMPOSITIONS AND METHODS FOR TREATING COGNITIVE IMPAIRMENT

(71) Applicant: UNITED STATES GOVERNMENT AS REPRESENTED BY THE DEPARTMENT OF VETERANS AFFAIR, Washington, DC (US)

(72) Inventor: Dongming Cai, Bronx, NY (US)

(21) Appl. No.: 18/272,844

(22) PCT Filed: Jan. 19, 2022
(86) PCT No.: PCT/US2022/012922

§ 371 Date: Jul. 18, 2023

Related U.S. Application Data
(60) Provisional application No. 63/139,083, filed on Jan. 19, 2021.

(51) Int. Cl. C12N 15/113; A61K 31/4422; A61P 25/28; C12Q 1/6883

(52) U.S. Cl. CPC C12N 15/113; A61K 31/4422; A61P 25/28; C12Q 1/6883; C12N 2310/141; C12Q 2600/158; C12Q 2600/178

(57) ABSTRACT

The disclosure relates to compositions and methods of treating mild cognitive impairment in a subject. The method also comprises administering to a subject in need of treatment an effective amount of miR-195, miR-195-5p, miR-195-3p, or fragments or variants thereof

### CROSS REFERENCE TO RELATED APPLICATIONS

This application claims the benefit of U.S. Provisional Application No. 63/139,083, filed Jan. 19, 2021. The content of this earlier filed application is hereby incorporated by reference herein in its entirety.

### STATEMENT REGARDING FEDERALLY FUNDED RESEARCH

This invention was made with government support under BX003380 awarded by the United States Department of Veterans Affairs. The government has certain rights in the invention.

### INCORPORATION OF THE SEQUENCE LISTING

The present application contains a sequence listing that is submitted via EFS-Web concurrent with the filing of this application, containing the file name “37759_0347P1_Sequence_Listing.txt” which is 4,096 bytes in size, created on Jan. 5, 2022, and is herein incorporated by reference in its entirety.

### BACKGROUND

Neurodegenerative disorders, such as Alzheimer's disease (AD), are a pervasive and growing problem. Neurodegenerative disorders relate to conditions that affect neurons, which can be damaged or destroyed in these disorders. Since neurons typically cannot regenerate, these conditions lead to often irreversible problems, resulting in problems with cognitive function, motor function, or both.

Alzheimer's disease, the most prevalent neurodegenerative disease of aging, affects one in eight older Americans. Recent evidence indicates that sporadic AD (which accounts for 90% of AD) is likely caused by an impaired Aβ clearance. Mild cognitive impairment (MCI) is a condition in which slight decreases are seen in cognitive function, such as memory (i.e., amnestic MCI) or thinking or language skills (or non-amnestic MCI). The changes seen in MCI are noticeable, but do not generally interfere with daily activities of the afflicted person nor require assisted living; because of this, MCI is distinguished from dementia.

Certain calcium channel blockers possess characteristics that suggest an approach to treating neurodegenerative disorders. Unfortunately, however, calcium channel blockers exhibit a number of side effects that would be disadvantageous in treating neurodegenerative diseases.

Therefore, effective and less toxic therapeutics are needed.

### SUMMARY

Disclosed herein are methods of treating cognitive impairment in a subject, the methods comprising: administering to the subject a therapeutically effective amount of a composition comprising miR-195-5p or a fragment or variant thereof.

Disclosed herein are methods of treating cognitive impairment in a subject, the methods comprising: administering a composition comprising miR-195-5p to the subject, wherein the subject has been diagnosed with a cognitive impairment by: i) determining, in a sample obtained from the subject, the expression level of a miR-195-5p, and ii) comparing the expression level of the miR-195-5p in the sample obtained from the subject with the expression level of the miR-195-5p in a reference sample, wherein a lower expression level of the miR-195-5p in the sample obtained from the subject indicates a cognitive impairment in the subject.

Disclosed herein are methods of ameliorating one or more symptoms of cognitive impairment in a subject, the methods comprising administering to the subject a therapeutically effective amount of a composition comprising miR-195-5p or a fragment or variant thereof.

Disclosed herein are methods of reducing synaptojanin 1 (synj1) activity or expression in a subject, the methods comprising administering to the subject a therapeutically effective amount of a composition comprising miR-195-5p or a fragment or variant thereof.

Disclosed herein are methods of inhibiting synaptojanin 1 (synj1) activity or expression in a subject, the methods comprising administering to the subject a therapeutically effective amount of a composition comprising miR-195-5p or a fragment or variant thereof.

Disclosed herein are methods of increasing amyloid β-protein (Aβ) clearance in a subject, the methods comprising administering to the subject a therapeutically effective amount of a composition comprising miR-195-5p or a fragment or variant thereof.

Disclosed herein are methods of reducing traumatic brain injury (TBI)-induced elevation in tau hyper-phosphorylation in a subject, the methods comprising administering to the subject a therapeutically effective amount of a composition comprising miR-195-5p or a fragment or variant thereof.

Disclosed herein are methods of reducing amyloid plaque burden in a subject, the methods comprising administering to the subject a therapeutically effective amount of a composition comprising miR-195-5p or a fragment or variant thereof.

Disclosed herein are methods of reducing tau hyper-phosphorylation in a subject, the methods comprising administering to the subject a therapeutically effective amount of a composition comprising miR-195-5p or a fragment or variant thereof.

Disclosed herein are methods of reducing IL-6 or TNFα release in a subject, the methods comprising administering to the subject a therapeutically effective amount of a composition comprising miR-195-5p or a fragment or variant thereof.

Disclosed herein are methods of decreasing phosphorylated tau production in a subject, the methods comprising administering to the subject a therapeutically effective amount of a composition comprising miR-195-5p or a fragment or variant thereof.

Disclosed herein are methods of treating ischemia induced microglial dysfunction and neuronal injury in a subject, the methods comprising administering to the subject a therapeutically effective amount of a composition comprising miR-195-5p or a fragment or variant thereof.

Disclosed herein are methods of rescuing Alzheimer's disease-related lysosomal defects in a subject, the methods comprising administering to the subject a therapeutically effective amount of a composition comprising miR-195-5p or a fragment or variant thereof.

Disclosed herein are methods comprising: (a) obtaining or having obtained a cerebrospinal fluid sample from a subject; (b) measuring the expression level of miR-195-5p in the cerebrospinal fluid sample; (c) identifying the subject as being in need for treatment with a composition comprising miR-195-5p when the level of miR-195-5p is lower than a level of miR-195-5p in a control sample; and (d) administering a composition comprising miR-195-5p or a fragment or variant thereof to the subject identified as in need of treatment.

Disclosed herein are methods comprising: (a) obtaining or having obtained a plasma or serum sample from a subject; (b) measuring the expression level of miR-195-5p in the plasma or serum sample; (c) identifying the subject as being in need for treatment with a composition comprising miR-195-5p when the level of miR-195-5p is lower than a level of miR-195-5p in a control sample; and (d) administering a composition comprising miR-195-5p or a fragment or variant thereof to the subject identified as in need of treatment.

Disclosed herein are methods of diagnosing a subject with a cognitive impairment, the methods comprising: a) measuring the expression level of miR-195-5p in a sample obtained from the subject; b) determining the subject has said cognitive impairment if the expression level of miR-195-5p is lower than the expression level of miR-195-5p of a reference sample, wherein the corresponding reference value is the average value of the expression level of miR-195-5p in healthy subjects; and c) treating the subject for said cognitive impairment.

Disclosed herein are methods of determining whether a subject has a cognitive impairment, the methods comprising: a) detecting the expression level of miR-195-5p in a sample obtained from the subject; b) comparing the expression level of miR-195-5p in the sample from the subject to the expression level of miR-195-5p from a reference sample; and c) determining the subject does not have a cognitive impairment when the expression level of miR-195-5p in the subject's sample is the same or higher than the expression level of miR-195-5p from a reference sample or determining the subject does have a cognitive impairment when the expression level of miR-195-5p in the subject's sample is lower than the level of miR-195-5p from the reference sample.

Other features and advantages of the present compositions and methods are illustrated in the description below, the drawings, and the claims.

### BRIEF DESCRIPTION OF THE DRAWINGS

FIGS. 1A-C show that miR-195 is identified as a top miRNA candidate involved in APOE-regulated synj1 expression. FIG. TA shows a Venn diagram showing that miR-195 as the miRNA in common shared among 4 groups. miRNAs differentially expressed between ApoE4⁺ and ApoE4⁻ carriers in the human ROSMAP dataset, miRNAs differentially expressed between ApoE4⁺ and ApoE4⁻ in the mouse miRNA array studies, miRNAs negatively correlated with synj1 mRNA in ROSMAP, and miRNAs predicted to target at synj1 mRNA by multiMiR database. Numbers of miRNAs overlapping among subgroups are indicated (red numbers). FIG. 1B shows Log Fold of changes (Log FC) and p values of differences in miR-195 levels between ApoE4⁺ and ApoE4⁻ carriers, between female ApoE4⁺ and ApoE4⁻ carriers in ROSMAP dataset, as well as differences in miR-195 levels between mouse ApoE4⁺ and ApoE4⁻ treated neurons. FIG. 1C shows the analysis of correlation between miR-195 and synj1 mRNA in human subjects of the ROSMAP database.

FIGS. 2A-D show that reduction of brain miR-195 levels in human brain and CSF samples is associated with ApoE4 genotype, disease progression, and cognitive decline. FIG. 2A shows that the amounts of miR-195 (presented as Log₂ fold changes) in human parietal cortex tissue of ApoE4^{+/-} subjects (CDR0.5-1) were lower than those in ApoE4^{-/-} subjects. N=17-18/group; log₂FC fold of changes: ApoE4^{-/-} 0.054±0.113 versus ApoE4^{+/-} 0.570±0.178, **p<0.01 with independent-samples t-tests. FIG. 2B shows the pattern of reduction in miR-195 levels (presented as Log₂ fold changes) along with AD disease progression from normal aging to MCI and early AD. N=12-19/group; log₂FC: 1.626±0.696 in CDR 0 subjects versus 0.242±0.104 in CDR 0.5 MCI patients; versus −0.663±0.135 in CDR 1 AD subjects; *p<0.05, ****p<0.0001 with ANOVA tests. FIG. 2C shows a positive correlation between CSF miR-195 levels and MMSE scores (r=0.455, p=0.029; N=23). FIG. 2D shows a negative correlation between CSF miR-195 and total tau levels (r=−0.408, p=0.04; N=23).

FIGS. 3A-D show that miR-195 expression is reduced in hippocampal brain tissue and cultured primary neurons of ApoE4 mice; modulating miR-195 levels regulates synaptojanin 1 expression. FIG. 3A shows that levels of miR-195 were reduced in 12-month old ApoE4 hippocampal brain tissue (log₂FC: −0.283±0.069) when compared to those in ApoE3 mice (log₂FC: −0.036±0.034). N=11-13/group with both males and females; ** p=0.0096 with ANOVA tests. A nominal reduction in miR-195 levels was seen in ApoE^{-/-} brains with no statistical significance (log₂FC: −0.12510.067, p=0.48). FIG. 3B shows that levels of miR-195 in ApoE^{-/-} neurons treated with ApoE4-CM were reduced (log₂FC=−0.314±0.073,) when compared to levels of those treated with ApoE3-CM (log₂FC: 0.184±0.094). N=5/group; **p=0.003 with independent-samples t-tests. FIG. 3C shows that differences in miR-195 expression levels between ApoE3-CM and ApoE4-CM treated neurons were abolished in the presence of RAP. The treatment of RAP in the presence of ApoE3-CM led to a reduction in miR-195 levels (log₂FC: −1.648±0.125; p<0.0001), whereas in ApoE4-CM treated conditions, miR-195 levels were much lower at baseline with a trend of improvement in the presence of RAP treatment (ApoE4 CM+BSA log₂FC: −3.193±0.144 versus ApoE4 CM+RAP log₂FC: −2.678±0.054; p=0.052). N=3/group; ****p<0.0001 by One-Way ANOVA tests. FIG. 3D shows that synj1 protein levels were reduced with miR-195 over-expression but not miR-374 over-expression in ApoE^{-/-} hippocampal neurons in the presence of ApoE4-CM. N=4/group; synj1 levels with miR-195: 62.87±4.48% of controls, **p=0.001; with miR-374: 102.4±7.77% of controls, p=0.93.

FIGS. 4A-F shows that over-expression of miR-195 rescues cognitive deficits and ameliorates AD-associated pathologies in ApoE4 mouse models. FIG. 4A shows a preference index=(time exploring novel object)/(time exploring novel object+time exploring familiar object) and discrimination index=(time exploring novel object-time exploring familiar object)/(time exploring novel object+time exploring familiar object) in 4 groups of mice: ApoE4^{+/-} scramble injection, ApoE4^{+/+} miR-195 injection, ApoE3^{+/+} scramble injection, and ApoE3^{+/+} miR-195 injection. N=19-23/group with both males and females; *p<0.05 with ANOVA tests. FIG. 4B shows the levels of pTau in KI mouse hippocampus. N=8/group with both males and females; *p<0.05 ** p<0.01 with ANOVA tests. FIG. 4C shows a preference index and discrimination index in 8 groups of mice: ApoE4^{+/+} FAD male scramble injection, ApoE4^{+/+} FAD male miR-195 injection, ApoE4^{+/+} FAD female scramble injection, ApoE4^{+/+} FAD female miR-195 injection, ApoE3^{+/+} FAD male scramble injection, ApoE3^{+/+} FAD male miR-195 injection, ApoE3^{+/+} FAD female scramble injection, and ApoE3^{+/+} FAD female miR-195 injection. N=6-10/group; *p<0.05 with ANOVA tests. FIGS. 4D and 4E show the levels of pTau (FIG. 4D) and oligomer Aβ₄₂ (FIG. 4E) in EFAD mouse hippocampus. N=6/group with both males and females; *p<0.05 ***p<0.001 ****p<0.0001 with ANOVA tests. FIG. 4F shows amyloid plaque burden in EFAD mouse hippocampus. Amyloid plaque load is quantified by density measured as area of plaques per mm² of brain region, as well as total numbers of plaques/μm² in 4 groups of mice: ApoE4^{+/+} FAD scramble injection, ApoE4^{+/+} FAD miR-195 injection, ApoE3^{+/+} FAD scramble injection, and ApoE3^{+/+} FAD miR-195 injection. N=3/group; *p<0.05 **p<0.01 with ANOVA tests.

FIGS. 5A-C show that over-expression of miR-195 rescues lysosomal defects in ApoE4 iPSC-derived brain cells. FIG. 5A shows the quantification of the lysosomes by size of iPSC-derived neuron and astrocyte co-culture of ApoE4^{+/+} normal aging (NA) and ApoE4^{+/+} AD subjects with various conditions: scramble control (ctrl), miR-195, and miR-195 inhibitor (miR inh). Alternatively, ApoE3^{+/+} and ApoE4^{+/+} N=3-6/conditions. FIG. 5B shows the quantification of the average size of the lysosomes of neurons in each experimental condition, the distribution of lysosome sizes/cell (measured by diameters; 0-10 μm, 10-20 μm, 20-30 μm and >30 μm), as well as the number of lysosomes in each cell (grouped by 1-5, 6-10, 11-15, 16-20 and >20 lysosomes/cell). FIG. 5C shows the quantification of the lysosomes by size (measured by areas; μm²) of 60-90 neurons (MAP-2⁺) in each experimental condition, the distribution of lysosome sizes/cell (measured by diameters; 0-10 μm, 10-20 μm, 20-30 μm and >30 μm), as well as the number of lysosomes in each cell (grouped by 1-5, 6-10, 11-15, 16-20 and >20 lysosomes/cell). ****p<0.00001 with ANOVA tests.

FIGS. 6A-B shows miR195 is identified as a top miRNA candidate involved in APOE-regulated synj1 expression. FIG. 6A shows that among 30 differentially expressed miRNAs between ApoE3 or ApoE4-conditioned media (CM) treated ApoE^{-/-} hippocampal neurons in microarray analysis, 15 are down-regulated in ApoE4 conditions. FIG. 6B shows the predicting scores of miR-195 targeted at synj1 mRNA using human and mouse multiMiR database.

FIGS. 7A-D show that reduction of brain miR-195 levels in human brain and CSF samples is associated with ApoE4 genotype, disease progression, and cognitive decline. FIG. 7A shows significant reduction in miR-195 levels in female subjects were seen compared to male subjects (log₂FC: 0.284±0.141 in male subjects versus −0.343±0.123 in female subjects, p=0.008), with differences also noted between male ApoE4^{-/-} subjects versus female ApoE4^{+/-} subjects (log₂FC: 0.340±0.120 in male ApoE4^{-/-} subjects versus −0.598±0.175 in female ApoE4^{+/-} subjects, p=0.02). FIG. 7B shows that reciprocal elevation of synj1 mRNA levels was seen in ApoE4^{+/-} subjects when compared to levels in ApoE4^{-/-} subjects (log₂FC: ApoE4^{-/-} 1.073±0.286 versus ApoE4^{+/-} 2.093±0.310, p=0.02). FIG. 7C shows a positive correlation between brain miR-195 and PIP₂ levels in ApoE4^{-/-} carriers with CDR 0.5-1 (r=0.472 p=0.048; N=18) with a positive correlation trend in CDR 0-1 subjects regardless of ApoE genotypes (r=0.283 p=0.06). FIG. 7D shows a negative correlation between brain miR195 and BACE-1 expression in the CDR 0.5-1 cohort (r=−0.52 p=0.004).

FIGS. 8A-C show that reduction of brain miR-195 levels is associated with ApoE4 genotype, disease progression and cognitive decline in human brain and CSF samples. FIG. 8A shows that amounts of miR-374 in human parietal cortex tissue of ApoE4^{+/-} subjects (CDR0.5-1) were lower than those in ApoE4^{-/-} subjects. N=17-18/group; log₂FC: ApoE4^{-/-} 0.285±0.105 versus ApoE4^{+/-} −0.306±0.171, **p<0.01 with independent-samples t-tests. However, along the disease progression, there was a transient elevation in miR-374 levels at MCI stage but no significant differences were seen between CDR 0 (normal aging) and CDR1 (early AD) cohorts (log₂FC: −1.160±0.830 in CDR0 subjects versus 0.299±0.098 in CDR 0.5 MCI patients, p=0.04). FIG. 8B shows that higher miR-155 levels seen in ApoE4^{+/-} subjects (log₂FC: ApoE4^{+/-} −2.275±0.280 versus ApoE4^{+/-} −1.107±0.451, p=0.035). FIG. 8C shows a positive correlation between CSF miR-195 and cardiolipin levels (r=0.684, p=0.0003; N=23).

FIGS. 9A-D show that miR-195 expression is reduced in hippocampal brain tissue and cultured primary neurons of ApoE4 mice; modulating miR-195 levels regulates synaptojanin 1 expression. FIG. 9A shows that levels of miR-374 were reduced in 12-month old ApoE4 hippocampal brain tissue (Log₂FC −0.455±0.098) when compared to those in ApoE3 mice (Log 2FC −0.026±0.050, p=0.02). A nominal reduction in miR-374 levels were seen in ApoE−/− mouse brains but without statistical significance (Log 2FC −0.197±0.124, p=0.40). N=8-10/group. A nominal difference was noted in miR-374 levels in neurons treated with ApoE4 CM (log₂FC: −0.037±0.245) when compared to those treated with ApoE3 CM (log₂FC: 0.328±0.254; p=0.33) but without statistical significance due to large variations among samples. N=5/group. FIG. 9B shows no changes in dyn protein levels in neurons over-expressing miR-195 or miR-374 (miR-195 over-expression 97.7% of controls; miR-374 over-expression 107.2% of controls). N=4/group. A representative example of western blot studies is shown. FIG. 9C shows that over-expression of miR-195 in ApoE3+/+ or ApoE4+/+ neurons reduced synj1 mRNA levels. ApoE4+/+ neurons exhibited more dramatic changes in synj1 mRNA levels with over-expression of miR-195 when compared to ApoE3+/+ (ApoE3+/+ w miR-195 log₂FC: −1.084±0.035 versus ApoE4+/+ w miR-195 log₂FC: −7.751±0.043). ****p<0.0001 with independent-samples t-tests. FIG. 9D shows that over-expression of miR-195 in ApoE3+/+ or ApoE4+/+ neurons reduced synj1 protein levels. Again, ApoE4+/+ neurons exhibited more dramatic changes in synj1 protein levels with over-expression of miR-195 when compared to changes in ApoE3+/+ neurons (ApoE3+/+ w miR-195 70.9±21.2% versus ApoE4+/+ w miR-195 48.0±9.84% of controls). **p=0.01 with independent-samples t-tests.

FIGS. 10A-F show that over-expression of miR-195 rescues cognitive deficits and ameliorates AD-associated pathologies in ApoE4 mouse models. FIG. 10A shows that levels of synj1 mRNA and protein levels are reduced in ApoE4+/+ mouse brains with miR-195 over-expression. ApoE4+/+ scramble controls versus ApoE4+/+ miR-195: synj1 mRNA Log 2FC 0.52 versus −1.29; **p=0.0005. synj1 protein 77.7 versus 56.4% of control; **p=0.006. Trends of reduction with lesser degrees in synj1 mRNA and protein levels were seen in ApoE3+/+ mouse brains with miR-195 over-expression. FIG. 10B shows that no significant changes in endogenous mouse Aβ40 or Aβ42 levels with over-expression of miR-195 in ApoE4+/+ or ApoE3+/+ brains were observed. FIG. 10C shows that no significant changes in ApoE levels with over-expression of miR-195 in ApoE4+/+ or ApoE3+/+ mouse brains were present. However, ApoE levels are much higher in ApoE3+/+ mouse brains after miR-195 over-expression when compared to those in ApoE4+/+ control or miR-195 injection mice. *p<0.05 with ANOVA tests. FIG. 10D shows that elevated miR-195 levels in both ApoE4+/+ and ApoE3+/+ mouse brains after viral manipulations are confirmed by qPCR. *p<0.05, ****p<0.00001 with ANOVA tests. FIG. 10E shows a representative example of western blot analysis of pTau, total Tau, synj1 protein, and R-actin in E4FAD and E3FAD mouse brains without or with miR-195 manipulation is shown. FIG. 10F shows that no significant changes are seen in soluble Aβ40 and Aβ42 levels in E4FAD and E3FAD mouse brains without or with miR-195 manipulation. However, E4FAD mouse brains exhibit higher levels of soluble Aβ40 when compared to levels in E3FAD mice regardless of miR-195 manipulation.

FIGS. 11A-F show that over-expression of miR-195 rescues lysosomal defects in ApoE4 iPSC-derived brain cells. FIG. 11A shows a representative example of pTau staining (AT8) in iPSC-derived neuron and astrocyte co-culture after control, miR-195 or miR-195 inhibitor transfection. Quantification of immunofluorescence intensity shown in bottom panels. *p<0.05 with ANOVA tests. FIG. 11B shows a Western blot analysis of pTau and synj1 protein levels of iPSC-derived brain cell culture from ApoE3+/+ normal aging (NA) and ApoE4+/+ AD subjects with scramble control (ctrl) or miR-195 transfection. FIG. 10C shows miR-195 levels in cultured iPSC-derived astrocytes from ApoE3+/+ normal aging (NA) and ApoE4+/+ AD subjects. **p<0.01 with independent-samples t-tests. FIG. 11D shows representative examples of immunofluorescence co-staining of a neuronal marker MAP-2 (red fluorescence), an astrocyte marker GFAP (green fluorescence) and DAPI (blue fluorescence), as well as immunofluorescence co-staining of an astrocyte marker GFAP (green fluorescence), lysosomes (Lysotracker: red fluorescence) and DAPI (blue fluorescence) of iPSC-derived neuron and astrocyte co-culture. Quantification of the lysosomes by size (measured by areas; μm²) of 60-90 astrocytes (GFAP+) in each experimental condition. *p<0.05, ***p<0.001, ****p<0.00001 with ANOVA tests. FIG. 11E shows quantification of the distribution of lysosome sizes per astrocyte (measured by diameters; 0-10 μm, 10-20 μm, 20-30 μm and >30 μm), as well as the number of lysosomes in each astrocyte (grouped by 1-5, 6-10, 11-15, 16-20 and >20 lysosomes/cell). FIG. 11F shows the Quantification of the lysosomes by size (measured by areas; μm²) of 70-100 neurons (MAP2+) in each experimental condition: synj1+/++control, synj1+/++miR-195; synj1−/−+control, and synj1−/−+miR-195. ****p<0.00001 with ANOVA tests.

FIGS. 12A-C show ischemia induced changes in miR-195 in exosomes derived from astrocytes. FIG. 12A shows that there were trends of reduction in miR-195 levels in ApoE4/4-derived exosomes, more so in ischemic conditions when compared to levels in ApoE3/3-derived exosomes. Levels of (FIG. 12B) pTau and (FIG. 12C) synj1 in exosomes of ApoE4/4 astrocytes trended higher than those in ApoE3/3 derived exosomes, more so with ischemic conditions. N=3.

FIGS. 13A-E show over-expression of miR-195 in microglia. FIG. 13A shows overexpression of miR-195 in microglia inhibits LPS-induced increases in expression of pdcd4 and smad7. *p<0.05, ***p<0.001, ****p<0.0001, N=8. FIG. 13B shows overexpression of miR-195 in microglia attenuates LPS-induced IL-6 and TNFα release. *p<0.05. N=4. FIG. 13C shows that overexpression of miR-195 in microglia augments anti-inflammatory responses with increased il10a gene expression. *p<0.05, ***p<0.001, N=8. FIG. 13D shows exosome miR-195 levels. FIG. 13E shows IL-6 and TNFα levels in LPS-treated BV2 cells in the presence of various exosomes.*p<0.05. N=4.

FIGS. 14A-B show exosomal miR-195 can rescue ischemia induced changes in microglia and neurons. FIG. 14A shows that levels of IL-6 was higher in ischemic ApoE4/4/microglia exposed to ApoE4/4-derived exosomes, but reduced significantly when exposed to ApoE4/4 over-expressing miR-195 astrocyte-derived exosomes. FIG. 14B shows that levels of pTau in ischemic ApoE4/4 neurons were reduced significantly in the presence of exosomes derived from miR-195-over-expressing astrocytes. N=4-5.*p<0.05; **p<0.01.

FIGS. 15A-E show changes in microglia-specific gene profiles with miR-195 over-expression in E4FAD mouse brain. FIG. 15A shows tSNE (t-distributed stochastic neighbor embedding) plot of brain cells sorted from EFAD with control and miR-195 (treatment), colored by cluster assignment based on cluster gene marker expression (N=4 pooled mice per condition). Cluster 0 is microglia-enriched cluster. FIG. 15B shows the top GO pathways enriched with cluster-specific DEGs through Fisher's exact test of gene set overlaps. FIG. 15C shows that sub clustering of the microglial cluster (C0) identified three major subsets. FIG. 15D shows that GO pathway enrichment analysis suggest different gene signatures in each microglia sub-cluster. FIG. 15E shows that studies of top GO pathways enriched with sub-cluster DEGs suggest that miR-195 over-expression down-regulates innate immune system and effector responses in Mic.C0 and Mic.C2 sub-clusters, as well as translation and ribosome activities in Mic.C1 sub-cluster, and up-regulates genes involved in oxidative phosphorylation and ATP metabolic processes in three sub-clusters.

FIGS. 16A-C show APOE4⁺ microglia with reduced miR-195 and increased synj1 expression manifest with impaired phagocytic activities and lysosomal enlargement that can be rescued by synj1 haploinsufficiency. FIG. 16A show the miR-195 levels in APOE4^{+/+} vs APOE3^{+/+} microglia; results presented as log₂FC, ***p=0.0005, N=3. FIG. 16B show the synj1 protein expression in APOE4^{+/+} vs APOE3^{+/+} synj1^{+/+} and synj1^{+/-} microglia; results presented as % of control with 100% as synj1 levels in APOE3^{+/+} synj1^{+/+} cells. *p<0.05. N=3. FIG. 16C show myelin uptake assays in APOE4^{+/+} (circle and square curves) vs APOE3^{+/+} (triangle curves) synj1^{+/+} (solid curves) and synj1^{+/-} (dashed curves) microglia. X-axis represents the incubation time (in hours) and Y-axis represents the amount of fluorescence signals (myelin taken up into the cells). Impaired myelin uptake in APOE4^{+/+} synj1^{+/+} microglia is rescued by synj1 haploinsufficiency (APOE4^{+/+} synj1^{+/+}). Note that APOE4^{+/-} synj1^{+/+} signals statistically lower than other groups (*p<0.05). Data are representative of more than two independent experiments.

FIGS. 17A-C show that over-expression of miR-195 in microglia (FIG. 17A) inhibits LPS-induced increases in expression of pdcd4 and smad7, *p<0.05, ***p<0.001, ****p<0.0001, N=8; (FIG. 18A) attenuates LPS-induced IL-6 and TNFα release, *p<0.05. N=4; and (FIG. 17C) augments anti-inflammatory responses with increased il10a gene expression. *p<0.05, ***p<0.001, N=8.

FIGS. 18A-B show that exosomal miR-195 uptake into microglia modulates inflammatory responses. FIG. 18A shows that exosome miR-195 is increased with over-expression of miR-195 in E4 astrocytes. FIG. 18B shows IL-6 and TNFα levels in LPS-treated microglia with exosomes (ADE from cultured APOE3/3 or 4/4 astrocytes without or with miR-195 over-expression). *p<0.05. N=4.

FIGS. 19A-C show the results of the evaluation of brain and serum exosomal miR-195 levels. FIG. 19A show the levels of miR-195 in brain and serum exosomes of 9-10 months old ApoE3 synj1^{+/+} and synj1^{+/-} and ApoE4 synj1^{+/+} and synj1^{+/-} mice. *p<0.05, ***p<0.001. N=3. FIG. 19B shows that the levels of serum exosomal miR-195 were significantly elevated in Cpd #6 or Cpd #9-treated 5×FAD mice when compared to vehicle controls. *p<0.05, **p<0.01. N=6-8/group. FIG. 19C shows that in the drug treatment cohorts, serum exosomal miR-195 levels were positively correlated with brain exosomal miR-195 as well as cognitive performance as measured by NOR preference index (0-1) and Y maze SAP scores (0-1), and reversely correlated with brain insoluble pTau and synj1 protein levels. *p<0.05.

### DETAILED DESCRIPTION

The present disclosure can be understood more readily by reference to the following detailed description of the invention, the figures and the examples included herein.

Before the present compositions and methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

As used in the specification and the appended claims, the singular forms “a,” “an” and “the” include plural referents unless the context clearly dictates otherwise.

The word “or” as used herein means any one member of a particular list and also includes any combination of members of that list.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, the subject is a human. The term does not denote a particular age or sex. Thus, adult, child, adolescent and newborn subjects, as well as fetuses, whether male or female, are intended to be covered.

The terms “subject” or “subject in need thereof” or “patient” are used interchangeably herein. These terms refer to a patient who has been diagnosed with the underlying disease or disorder to be treated. The subject may currently be experiencing symptoms associated with the disease or disorder or may have experienced symptoms in the past. Additionally, a “subject in need thereof” may be a patient at risk of developing a particular disease, or to a patient reporting one or more of the physiological systems of a disease, even though a diagnosis of this disease may not have been made. As a non-limiting example, a “subject in need thereof”, for purposes of this application, may include a patient who is currently diagnosed with cognitive impairment or was diagnosed with a cognitive impairment in the past, regardless of current symptomatology. A “subject in need thereof” can also include a patient who is showing cognitive deficits, but has not been diagnosed with a particular disease or disorder.

As used herein, the term “patient” refers to a subject afflicted with a disease or disorder. The term “patient” includes human and veterinary subjects. In some aspects of the disclosed methods, the “patient” has been diagnosed with a need for treatment for cognitive impairment, such as, for example, prior to the administering step.

As used herein, the term “treating” or “treatment” refers to partially or completely alleviating, ameliorating, relieving, delaying onset of, inhibiting or slowing progression of, reducing severity of, and/or reducing incidence of one or more symptoms or features of, or otherwise prevent, hinder, retard, or reverse the progression of a particular disease, disorder, and/or condition or other undesirable symptom(s). Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cognitive impairment.

The terms “administer”, “administering” or “administration” in reference to a dosage form of the invention refers to the act of introducing the dosage form into the system of subject in need of treatment. When a dosage form of the invention is given in combination with one or more other active agents (in their respective dosage forms), “administration” and its variants are each understood to include concurrent and/or sequential introduction of the dosage form and the other active agents. Administration of any of the described dosage forms includes parallel administration, co-administration or sequential administration. In some situations, the therapies are administered at approximately the same time, e.g., within about a few seconds to a few hours of one another.

A “therapeutically effective” amount of the compounds described herein is typically one which is sufficient to achieve the desired effect and may vary according to the nature and severity of the disease condition, and the potency of the compound. It will be appreciated that different concentrations may be employed for prophylaxis than for treatment of an active disease. A therapeutic benefit is achieved with the amelioration of one or more of the physiological symptoms associated with the underlying disorder such that an improvement is observed in the patient, notwithstanding that the patient may still be afflicted with the underlying disorder. The therapeutically effective amount can be the amount of the composition administered to a subject that leads to a full resolution of the symptoms of the condition or disease, a reduction in the severity of the symptoms of the condition or disease, or a slowing of the progression of symptoms of the condition or disease. The methods described herein can also include a monitoring step to optimize dosing. The compositions described herein can be administered as a preventive treatment or to delay or slow the progression of the condition or disease (e.g., cognitive impairment).

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also relate to a distemper, ailing, ailment, malady, disorder, sickness, illness, complaint, affection.

As used herein, the term “normal” refers to an individual, a sample or a subject that does not have a cognitive impairment.

The terms “vector” or “construct” refer to a nucleic acid sequence capable of transporting into a cell another nucleic acid to which the vector sequence has been linked.

The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

As used herein, the term “synergistic composition” refers to the application of the combination of miR-195, a fragment of miR-195, a variant of miR-195; miR-195-5p or a fragment or variant thereof; or miR-195-3p or a fragment or variant thereof, and an additional therapeutic agent. The synergistically effective amount refers to the amount of each component which, in combination, is effective, for example, in reducing or inhibiting synaptojanin 1 (synj1) activity or expression, increasing Aβ clearance, reducing traumatic brain injury (TBI)-induced elevation in tau hyper-phosphorylation, reducing amyloid plaque burden, reducing tau hyper-phosphorylation, or rescuing Alzheimer's disease-related lysosomal defects, and which produces a response greater than either component alone.

“Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels. As used herein, promoting can also mean enhancing.

As used herein, the terms “inhibit” or “inhibiting” or “reducing” refer to a reduction or decrease in an activity, response, condition, disease, or other biological parameter. This can include but is not limited to the initiation of the activity, response, condition, or disease. This may also include, for example, a 10% decrease in the activity, response, condition, or disease as compared to the native or control level. Thus, in some aspects, the decrease or reduction can be a 10, 20, 30, 40, 50, 60, 70, 80, 90, 100%, or more, or any amount of promotion in between compared to native or control levels. In some aspects, the decrease or reduction is 10-20, 20-30, 30-40, 40-50, 50-60, 60-70, 70-80, 80-90, or 90-100% as compared to native or control levels. In some aspects, the decrease or reduction is 0-25, 25-50, 50-75, or 75-100%, or more, such as 200, 300, 500, or 1000% more as compared to native or control levels. In some aspects, the decrease or reduction can be greater than 100 percent as compared to native or control levels, such as 100, 150, 200, 250, 300, 350, 400, 450, 500% or more as compared to the native or control levels.

The prefix “mir” followed by a hyphen and a number is often used to refer to microRNAs. It is common to differentiate the pre-miRNA from the mature form by capital letters, so that the abbreviation “mir-” corresponds to the pre-miRNA, while the abbreviation “miR-” indicates that a mature microRNA is referred to. An abbreviation making reference to the species is often used in the front; thus, for example “hsa” refers to human microRNAs, of Homo sapiens.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the D- or L-configuration. If non-naturally occurring side chains are used, non-amino acid substituents may be used, for example to prevent or retard in vivo degradation.

The term “fragment” can refer to a portion (e.g., at least 4, 5, 6, 7, 8, 9, 10, 11, 12, 13, 14, 15, 16, 17, etc. amino acids) of a peptide that is substantially identical to a reference peptide and retains the biological activity of the reference. In some aspects, the fragment or portion retains at least 50%, 75%, 80%, 85%, 90%, 95% or 99% of the biological activity of the reference peptide described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

Although the foregoing disclosure has been described in some detail by way of illustration and example for purposes of clarity of understanding, certain changes and modifications may be practiced within the scope of the appended claims.

The compounds described herein can be used to treat cognitive impairment (also referred to herein as “neurodegenerative disorder” or “neurodegenerative disease”), such as that found in Alzheimer's disease, other dementias (such as vascular dementia, frontotemporal dementia (FTD), Lewy body dementia (LDB), or mixed dementia), mild cognitive impairment (MCI), ischemic conditions (e.g., vascular dementia, cerebrovascular disease, and other ischemic processes) and Down Syndrome. The compounds described herein can be used to treat traumatic brain injury (TBI). There is a relationship between TBI, Alzheimer's disease (AD), and dementia. People who suffer a TBI are two- to four-times more likely to develop late-onset neurodegeneration and AD.

Alzheimer's disease (AD) is characterized neuropathologically by senile plaques containing β-amyloid peptides (Aβ), as well as neurofibrillary tangles consisting of hyperphosphorylated tau. Either overproduction or impaired clearance of Aβ can lead to Aβ accumulation. Recent evidence suggests that late-onset AD cases (accounting for 90% of AD cases) are correlated with an overall impairment in Aβ clearance. Furthermore, several studies report pathological changes of the endosomal/lysosomal network, which develop in neurons as Alzheimer's disease progresses, and include dysregulation of endocytosis and progressive failure of lysosomal clearance mechanisms. A close connection between lysosomal protein clearance failure and mechanisms of neurodegeneration is also well documented.

Endosomal anomalies are considered one of the earliest AD pathologies, and increased function of synj1 is linked to enlargement of early endosomes. Synaptojanin 1 (synj1) is the main phosphoinositol bisphosphate (PIP₂) degrading enzyme in the brain and synapses. Most importantly, it has been found that down-regulation of synj1 increases Aβ uptake and lysosomal trafficking, thereby stimulating AD clearance. Furthermore, reduction of synj1 attenuates amyloid-induced neuropathologic changes and behavior deficits in an AD transgenic mouse model.

The Alzheimer's Association estimates that MCI afflicts 15-20% of people 65 years of age or older. It is thought that the risk factors that lead to MCI are similar to those for dementia and Alzheimer's disease (AD); these include advancing age, cardiovascular disease (or risk factors leading to it), and/or familial history of dementia or AD. Additionally, individuals who have MCI are at an increased risk for developing AD or dementia than non-MCI-afflicted individuals. People who are carriers of the ApoE4 gene are also thought to be at a higher risk of developing MCI and/or AD. The compounds of the invention can be used to treat patients who carry the ApoE4 gene, patients with MCI, and/or patients with preclinical or active AD.

Down syndrome (DS) occurs once in approximately every 700 births in the United States and is caused by an extra copy of at least a portion of chromosome 21. Synaptojanin 1 has been implicated as being involved in Down syndrome, perhaps because its gene, SYNJ1, is believed to be located on human chromosome 21. Individuals affected with DS commonly develop Alzheimer's disease.

Traumatic brain injury (TBI) can also be treated by any of compounds disclosed herein. According to the Centers for Disease Control, TBI sufferers were seen in U.S. emergency rooms 2.5 million times in 2010. Neuropathological studies of human TBI cases have described the development of neurofibrillary tangles and amyloid plaques associated with neurodegenerative processes.

Without being held to any one theory, data suggest that ApoE proteins regulate changes in brain phospholipid homeostasis in response to blast TBI and that the ApoE4 isoform is dysfunctional in this process. Down-regulation of synj1 has been shown to rescue blast-induced phospholipid dysregulation and prevent development of Tau hyper-phosphorylation in ApoE4 carriers.

### Compositions

Disclosed herein are compositions for the treatment of cognitive impairment. In some aspects, the target gene can be the synaptojanin 1 (synj1) gene. In some aspects, the sample can express increased levels of synaptojanin 1 (synj1). In some aspects, the sample can express decreased levels of miR-195, miR-195-5p or miR-195-3p. In some aspects, the sample can express decreased levels of miR-195, miR-195-5p or miR-195-3p variants. In some aspects, the compositions can comprise a miR-195 and any of the compounds disclosed in Tables 1 and 2. In some aspects, the composition can be a synergistic composition.

In some aspects, the cognitive impairment can be Alzheimer's disease, mild cognitive impairment, Lewy body dementia (LBD), frontotemporal dementia (FTD), ischemic conditions (e.g., vascular dementia, cerebrovascular disease, and other ischemic processes), mixed dementia, or Down Syndrome. In some aspects, the cognitive impairment can be related to a traumatic brain injury, MicroRNAs (miRNAs) are monocatenary RNA molecules, normally of about 20-25 nucleotides, which have the ability to regulate the expression of specific genes by means of their post-transcriptional silencing, as a result of the miRNA joining the messenger RNA in a region in which they are complementary, the pairing leading to the degradation of the messenger RNA. The microRNAs are coded in the genome and are initially formed, as is known, as pri-miRNA, which is a long molecule of bicatenary RNA with the ability to form hairpins by complementarity between internal regions of the molecule. The so-called precursor microRNA, pre-miRNA, is formed when the pri-miRNA is processed by the drosha enzyme, which cuts off or eliminates the bases of the hairpins, that is, the unpaired ends. The pre-miRNA is transported from the nucleus to the cytoplasm, where it is fragmented by the dicer enzyme, which cuts it to the final length of 20-25 nucleotides, after which the resulting duplex is separated, resulting in two monocatenary RNAs, one of which is the mature microRNA, which performs its silencing action integrated in the RISC complex.

miR-195 is one of the miR-15/107 family members, which are stress inducible. Members of the miR-15/107 group have a similar sequence, AGCAGC, near the 5′ end of the mature miRNA, named AGCx2 miRNA. The human miR-195 gene originates from intron 7, which is located on chromosome 17p13.1 and on the reverse strand of the mRNA gene AK098506, encoding an unknown functional protein. The predicted stem-loop structure of miR-195 determined using the miRBase (http://mirbase.org/) is shown in Yu et al, Onco Targets Ther. 2018; 11: 7109-7123, which is hereby incorporated by reference for the sequence and structure of the predicted stem-loop structure of miR-195. The miR-195 (also referred to as ‘stem loop’) sequence is AGCUUCCCUGGCUCUAGCAGCACAGAAAUAUUGGCACAGGGAAGCGAGUCUGC CAAUAUUGGCUGUGCUGCUCCAGGCAGGGUGGUG (SEQ ID NO: 4). The miR-195 hairpin gives rise to the “guide strand” miR-195-5p (having the sequence UAGCAGCACAGAAAUAUUGGC; SEQ ID NO: 1) and the sister “passenger” strand miR-195-3p (having the sequence CCAAUAUUGGCUGUGCUGCUCC; SEQ ID NO: 2).

In some aspects, the miR-195 can be hsa-miR-195. In some aspects, the miR-195-5p can be hsa-miR-195-5p. In some aspects, the miR-195-5p can comprise the nucleotide sequence UAGCAGCACAGAAAUAUUGGC (SEQ ID NO: 1). In some aspects, the miR-195-3p can be hsa-miR-195-3p. In some aspects, the miR-195-3p can comprise the nucleotide sequence CCAAUAUUGGCUGUGCUGCUCC (SEQ ID NO: 2).

In some aspects, miR-195-5p can consist of the nucleotide sequence UAGCAGCACAGAAAUAUUGGC (SEQ ID NO: 1), In some aspects, miR-195-3p can consist of the nucleotide sequence CCAAUAUUGGCUGUGCUGCUCC (SEQ ID NO: 2). In some aspects, the composition can consist of a sequence derived from miR-195. In some aspects, the composition can consist of a sequence derived from miR-195, wherein the sequence derived from miR-195 has increased stability as compared to miR-195.

Disclosed herein are fragments of miR-195, miR-195-5p and miR-195-3p. As used herein, the term “fragment” refers to a portion of the full-length miR-195, miR-195-5p (SEQ ID NO: 1) or miR-195-3p (SEQ ID NO: 2). The size of the fragment can vary and must include a functional fragment, that is, the fragment must be able to modulate the activity or expression of synj1 or and have therapeutic utility against synj1 expressing cells as described herein. Typically, the fragment can comprise at least the seed region sequence AGCAGCA (SEQ ID NO: 3). In some aspects, the fragment can comprise at least the seed region sequence AGCAGCA (SEQ ID NO: 3).

Disclosed herein are also variants of miR-195, miR-195-5p and miR-195-3p. Variants of miR-195 (also referred to herein as miR-195 variants), miR-195-5p and miR-195-3p can include nucleotide sequences that are substantially similar to sequences of miR-195, miR-195-5p or miR-195-3p, respectively, as well as precursors or sequences derived thereof. miR-195, miR-195-5p and miR-195-3p variants must include a functional fragment, that is, the miR-195, miR-195-5p and miR-195-3p variant must be able to modulate the activity or expression of synj1 or and have therapeutic utility against synj1 expressing cells as described herein. Typically, the miR-195, miR-195-5p and miR-195-3p variant can comprise at least the seed region sequence of miR-195 AGCAGCA (SEQ ID NO: 3). In some aspects, the miR-195, miR-195-5p and miR-195-3p variants can comprise at least the seed region sequence AGCAGCA (SEQ ID NO: 3).

In some aspects, miR-195, miR-195-5p or miR-195-3p variants include nucleotide sequences that are substantially similar to the miR-195 sequence or fragments thereof, the miR-195-5p sequence or fragments thereof, or the miR-195-3p sequence or fragments thereof including the miR-195 seed sequence. miR-195, miR-195-5p and miR-195-3p variants can also include nucleotide sequences that are substantially similar to sequences of miRNA disclosed herein. A “variant” can mean a difference in some way from the reference sequence other than just a simple deletion of an N- and/or C-terminal nucleotide. Variants can also or alternatively include at least one substitution and/or at least one addition, there may also be at least one deletion. In some aspects, the variant miRNA to be administered can comprise a sequence displaying at least 80% sequence identity to the sequence of miR-195-5p (SEQ ID NO: 1), miR-195-3p (SEQ ID NO: 2), or miR-195 (SEQ ID NO: 4). In some aspects, the miRNA to be administered can comprise a sequence displaying at least 90% sequence identity to SEQ ID NO: 1, SEQ ID NO: 2 or SEQ ID NO: 4). In some aspects, the miRNA to be administered can comprise a sequence displaying at least 91%, 92%, 93%, 94%, 95%, 96%, 97%, 98% or 99% sequence identity to SEQ ID NO: 1, SEQ ID NO: 2 or SEQ ID NO: 4). Alternatively or in addition, variants can comprise modifications, such as non-natural residues at one or more positions with respect to the miR-195 sequence, the miR-195-5p sequence or the miR-195-3p sequence. In some aspects, the variant can be a sequence wherein the last nucleotide of the miRNA is changed. In some aspects, the variant can be a sequence comprising at least one, at least two, or at least three substitutions at the 5′ end of the miR-195. miR-195-5p, or miR-195-3p. In some aspects, nucleotide substitutions can include nucleotide substitutions to the reference sequence which increase stability of the miR-195, miR-195-5p, miR-195-3p or a variant thereof. In some aspects, nucleotide substitutions can be those which permit conjugation of the miR-195, miR-195-5p, miR-195-3p or a variant thereof to a polymer or copolymer for forming a nanoparticle. Nucleotide substitutions can be substitutions of one or two bases. In some aspects, nucleotide substitutions can be substitutions of three bases. Deletions and insertions can include from one (1) to about three (3) bases.

Substitutions, deletions, insertions or any combination thereof may be used to arrive at a final derivative or variant. Generally, these changes are done on a few nucleotides to minimize the alteration of the molecule. However, larger changes may be tolerated in certain circumstances.

Generally, the nucleotide identity between individual variant sequences can be at least 90%, 91%, 92%, 93%, 94%, 95%, 96%, 97%, 98%, 99%, or 100%. Thus, a “variant sequence” can be one with the specified identity to the parent or reference sequence of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence. For example, a “variant sequence” can be a sequence that contains 1, 2, 3, or 4 nucleotide base changes as compared to the parent or reference sequence of the invention, and shares or improves biological function, specificity and/or activity of the parent sequence. In some aspects, the parent or reference sequence can be 195.

In some aspects, any of sequences disclosed herein can include a single nucleotide change as compared to the parent or reference sequence. In some aspects, any of the sequences disclosed herein can include at least two nucleotide changes as compared to the parent or reference sequence. The nucleotide identity between individual variant sequences can be at least 60%, 65%, 70%, 75%, 80%, 85%, 90%, 91%, 92%, 93%, 94%, 95%, 96%, 97%, 98%, 99% or 100%. Thus, a “variant sequence” can be one with the specified identity to the parent sequence of the invention, and shares biological function, including, but not limited to, at least 80%, 81%, 82%, 83%, 84%, 85%, 86%, 87%, 88%, 89%, 90%. 91%, 92%. 93%, 94%, 95%, 96%, 97%, 98%, or 99% of the specificity and/or activity of the parent sequence. The variant sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent sequence.

In some aspects, the amino acid sequence of miR-195-5p, miR-195-3p or miR-195 described herein can include a peptide sequence that has some degree of identity or homology to any of sequences of miR-195-5p, miR-195-3p or miR-195 sequences disclosed herein. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The decoy peptides or polypeptides described herein can have at least or about 25%, 50%, 65%, 75%, 80%, 85%, 90%, 95%, 96%, 97%, 98%, or 99% identity or homology to the decoy peptide or polypeptide, wherein the decoy peptide or polypeptide is one or more of SEQ ID NOs: 1, 2 or 4.

Also disclosed herein are the compounds set forth in Tables 1 and 2. In some aspects, the compositions described herein can further include one or more of the compounds disclosed in Table 1 or Table 2. The compounds in Table 1 and 2 can be useful in any of the methods described herein.

TABLE 1
Compounds.

| Compound No. | Structure |
|---|---|
| 1 | 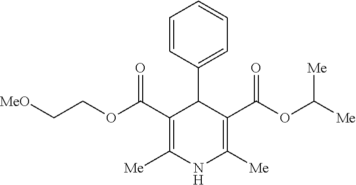 [SMILES: COCCOC(=O)C1=C(C)NC(C)=C(C(=O)OC(C)C)C1c1ccccc1] |
| 2 | 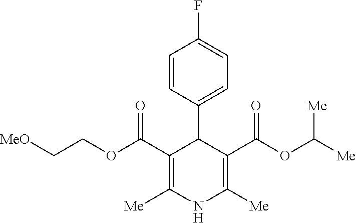 [SMILES: COCCOC(=O)C1=C(C)NC(C)=C(C(=O)OC(C)C)C1c1ccc(F)cc1] |
| 3 | 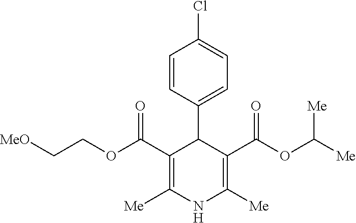 [SMILES: COCCOC(=O)C1=C(C)NC(C)=C(C(=O)OC(C)C)C1c1ccc(Cl)cc1] |
| 4 | 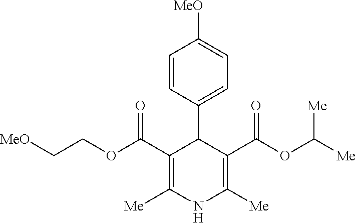 [SMILES: COCCOC(=O)C1=C(C)NC(C)=C(C(=O)OC(C)C)C1c1ccc(OC)cc1] |
| 5 | 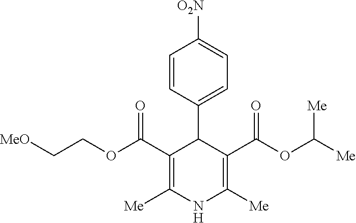 [SMILES: COCCOC(=O)C1=C(C)NC(C)=C(C(=O)OC(C)C)C1c1ccc([N+](=O)[O-])cc1] |
| 6 | 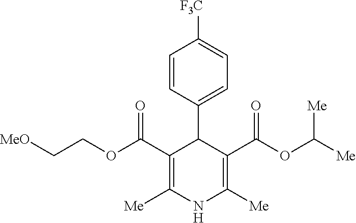 [SMILES: COCCOC(=O)C1=C(C)NC(C)=C(C(=O)OC(C)C)C1c1ccc(C(F)(F)F)cc1] |
| 7 | 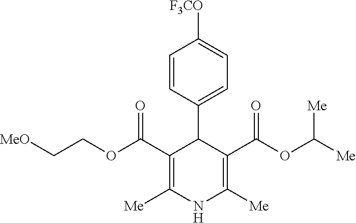 [SMILES: COCCOC(=O)C1=C(C)NC(C)=C(C(=O)OC(C)C)C1c1ccc(OC(F)(F)F)cc1] |
| 8 | 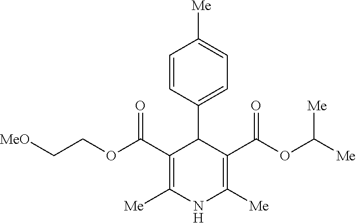 [SMILES: COCCOC(=O)C1=C(C)NC(C)=C(C(=O)OC(C)C)C1c1ccc(C)cc1] |
| 9 | 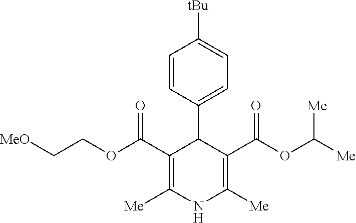 [SMILES: COCCOC(=O)C1=C(C)NC(C)=C(C(=O)OC(C)C)C1c1ccc(C(C)(C)C)cc1] |

Additional compounds useful in any of the methods described herein are disclosed in Table 2.

TABLE 2
Compounds.

| Compound ID | Structure |
|---|---|
| 20 | 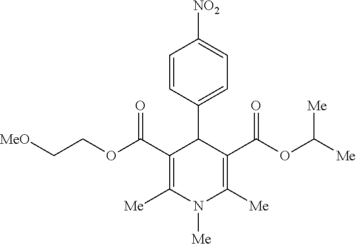 [SMILES: COCCOC(=O)C1=C(C)N(C)C(C)=C(C(=O)OC(C)C)C1c1ccc([N+](=O)[O-])cc1] |
| 21 | 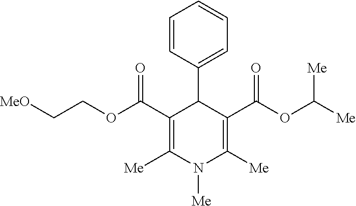 [SMILES: COCCOC(=O)C1=C(C)N(C)C(C)=C(C(=O)OC(C)C)C1c1ccccc1] |
| 22 | 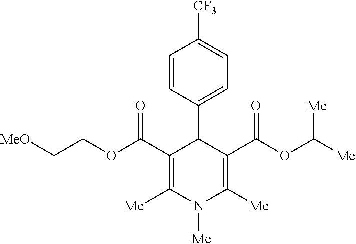 [SMILES: COCCOC(=O)C1=C(C)N(C)C(C)=C(C(=O)OC(C)C)C1c1ccc(C(F)(F)F)cc1] |
| 23 | 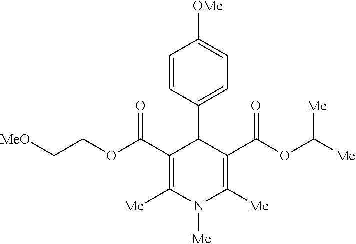 [SMILES: COCCOC(=O)C1=C(C)N(C)C(C)=C(C(=O)OC(C)C)C1c1ccc(OC)cc1] |
| 24 | 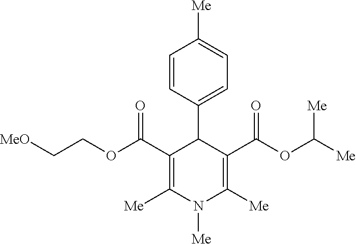 [SMILES: COCCOC(=O)C1=C(C)N(C)C(C)=C(C(=O)OC(C)C)C1c1ccc(C)cc1] |
| 25 | 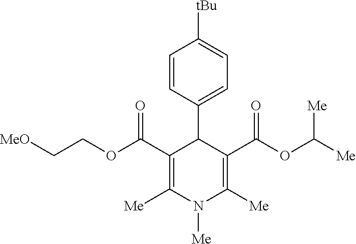 [SMILES: COCCOC(=O)C1=C(C)N(C)C(C)=C(C(=O)OC(C)C)C1c1ccc(C(C)(C)C)cc1] |
| 26 | 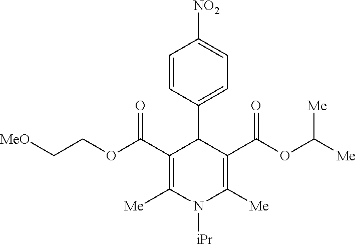 [SMILES: COCCOC(=O)C1=C(C)N(C(C)C)C(C)=C(C(=O)OC(C)C)C1c1ccc([N+](=O)[O-])cc1] |
| 27 | 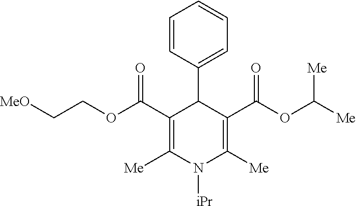 [SMILES: COCCOC(=O)C1=C(C)N(C(C)C)C(C)=C(C(=O)OC(C)C)C1c1ccccc1] |
| 28 | 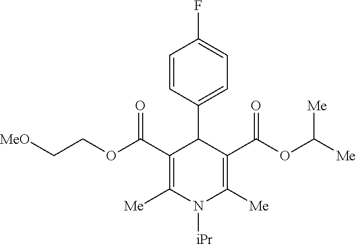 [SMILES: COCCOC(=O)C1=C(C)N(C(C)C)C(C)=C(C(=O)OC(C)C)C1c1ccc(F)cc1] |
| 29 | 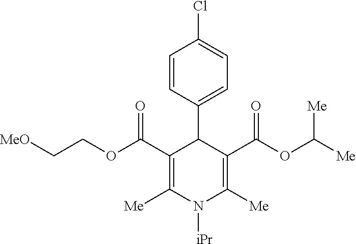 [SMILES: COCCOC(=O)C1=C(C)N(C(C)C)C(C)=C(C(=O)OC(C)C)C1c1ccc(Cl)cc1] |
| 30 | 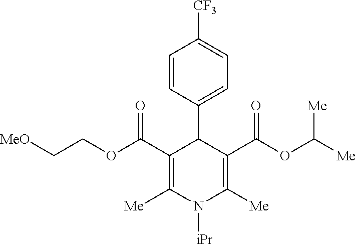 [SMILES: COCCOC(=O)C1=C(C)N(C(C)C)C(C)=C(C(=O)OC(C)C)C1c1ccc(C(F)(F)F)cc1] |
| 31 | 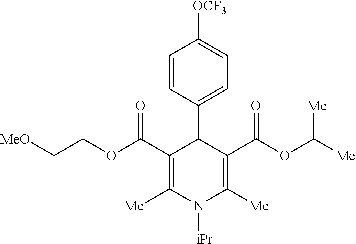 [SMILES: COCCOC(=O)C1=C(C)N(C(C)C)C(C)=C(C(=O)OC(C)C)C1c1ccc(OC(F)(F)F)cc1] |
| 32 | 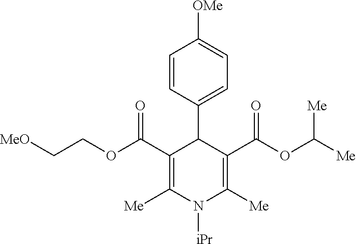 [SMILES: COCCOC(=O)C1=C(C)N(C(C)C)C(C)=C(C(=O)OC(C)C)C1c1ccc(OC)cc1] |
| 33 | 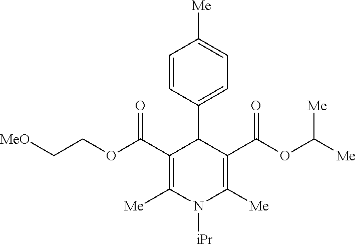 [SMILES: COCCOC(=O)C1=C(C)N(C(C)C)C(C)=C(C(=O)OC(C)C)C1c1ccc(C)cc1] |
| 34 | 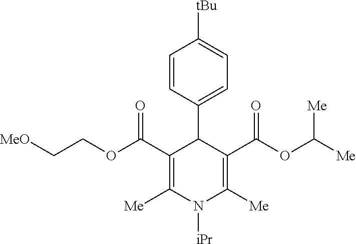 [SMILES: COCCOC(=O)C1=C(C)N(C(C)C)C(C)=C(C(=O)OC(C)C)C1c1ccc(C(C)(C)C)cc1] |
| 35 | 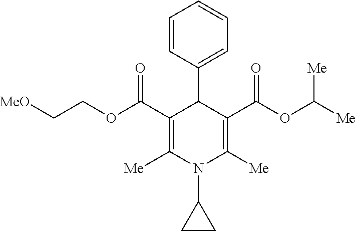 [SMILES: COCCOC(=O)C1=C(C)N(C2CC2)C(C)=C(C(=O)OC(C)C)C1c1ccccc1] |
| 36 | 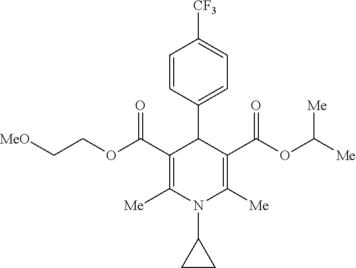 [SMILES: COCCOC(=O)C1=C(C)N(C2CC2)C(C)=C(C(=O)OC(C)C)C1c1ccc(C(F)(F)F)cc1] |
| 37 | 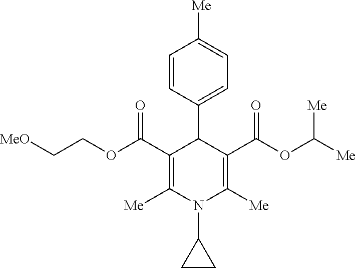 [SMILES: COCCOC(=O)C1=C(C)N(C2CC2)C(C)=C(C(=O)OC(C)C)C1c1ccc(C)cc1] |

### Methods

Disclosed herein, are methods of diagnosing a subject with a cognitive impairment. Also, disclosed herein, are methods of diagnosing and treating a subject with a cognitive impairment. In some aspects, the methods can comprise measuring the expression level of miR-195, miR-195-5p or miR-195-3p in a sample obtained from the subject. In some aspects, the methods can comprise measuring the expression level of miR-195, miR-195-5p or miR-195-3p variants in a sample obtained from the subject. In some aspects, the methods can comprise determining the subject has said cognitive impairment if the expression level of miR-195, miR-195-5p or miR-195-3p is lower than the expression level of miR-195, miR-195-5p or miR-195-3p of a reference sample, wherein the corresponding reference value is the average value of the expression level of miR-195, miR-195-5p or miR-195-3p in healthy subjects. In some aspects, the methods can comprise treating the subject for said cognitive impairment. In some aspects, the step of treating the subject for said cognitive impairment a comprise administering to the subject a therapeutically effective amount of a composition comprising miR-195, miR-195-5p or miR-195-3p. In some aspects, the step of treating the subject for said cognitive impairment a comprise administering to the subject a therapeutically effective amount of a composition comprising miR-195, miR-195-5p or miR-195-3p variants. In some aspects, the composition can comprise a polynucleotide comprising a sequence at least 90% sequence identity to miR-195, miR-195-5p, miR-195-3p.

Disclosed herein are methods of determining whether a subject has a cognitive impairment. In some aspects, the methods can comprise detecting the expression level of miR-195, miR-195-5p or miR-195-3p in a sample obtained from the subject. In some aspects, the methods can comprise comparing the expression level of miR-195, miR-195-5p or miR-195-3p in the sample from the subject to the expression level of miR-195, miR-195-5p or miR-195-3p from a reference sample. In some aspects, the methods can comprise detecting the expression level of miR-195, miR-195-5p or miR-195-3p variants in a sample obtained from the subject. In some aspects, the methods can comprise comparing the expression level of miR-195, miR-195-5p or miR-195-3p variants in the sample from the subject to the expression level of miR-195, miR-195-5p or miR-195-3p variants from a reference sample. In some aspects, the methods can comprise determining the subject does not have a cognitive impairment when the expression level of miR-195, miR-195-5p or miR-195-3p in the subject's sample is the same or higher than the expression level of miR-195, miR-195-5p or miR-195-3p from a reference sample or determining the subject does have a cognitive impairment when the expression level of miR-195, miR-195-5p or miR-195-3p in the subject's sample is lower than the level of miR-195, miR-195-5p or miR-195-3p from the reference sample. In some aspects, the methods can further comprise administering to the subject diagnosed with said cognitive impairment a therapeutically effective amount of a composition comprising miR-195, miR-195-5p or miR-195-3p. In some aspects, the methods can further comprise administering to the subject diagnosed with said cognitive impairment a therapeutically effective amount of a composition comprising miR-195, miR-195-5p or miR-195-3p variants. In some aspects, the composition can comprise a polynucleotide comprising a sequence at least 90% sequence identity to miR-195, miR-195-5p or miR-195-3p.

Further disclosed herein are methods comprising. (a) obtaining or having obtained a cerebrospinal fluid sample from a subject; (b) measuring the expression level of miR-195, miR-195-5p or miR-195-3p in the cerebrospinal fluid sample; (c) identifying the subject as being in need for treatment with a composition comprising miR-195, miR-195-5p or miR-195-3p when the level of miR-195, miR-195-5p or miR-195-3p is lower than a level of miR-195, miR-195-5p or miR-195-3p in a control sample; and (d) administering a composition comprising miR-195, miR-195-5p, miR-195-3p to the subject identified as in need of treatment. In some aspects, the subject has a cognitive impairment.

Also disclosed herein are methods comprising: (a) obtaining or having obtained a plasma or serum sample from a subject; (b) measuring the expression level of miR-195, miR-195-5p or miR-195-3p in the plasma or serum sample; (c) identifying the subject as being in need for treatment with a composition comprising miR-195, miR-195-5p or miR-195-3p when the level of miR-195, miR-195-5p or miR-195-3p is lower than a level of miR-195, miR-195-5p or miR-195-3p in a control sample; and (d) administering a composition comprising miR-195, miR-195-5p, miR-195-3p to the subject identified as in need of treatment. In some aspects, the subject has a cognitive impairment.

Also disclosed herein are methods of treating cognitive impairment in a subject. In some aspects, the methods can comprise administering a composition comprising miR-195, miR-195-5p or miR-195-3p to the subject, wherein the subject has been diagnosed with a cognitive impairment by: i) determining, in a sample obtained from the subject, the expression level of a miR-195, miR-195-5p or miR-195-3p ii) comparing the expression level of miR-195, miR-195-5p or miR-195-3p in the sample obtained from the subject with the expression level of miR-195, miR-195-5p or miR-195-3p from a reference sample, wherein a lower expression level of the miR-195, miR-195-5p or miR-195-3p in the sample obtained from the subject indicates a cognitive impairment in the subject. In some aspects, the reference sample can be obtained from a subject that does not have or has not been diagnosed as having said cognitive impairment.

Disclosed herein are methods of down-regulating innate immune system or effector responses in a subject comprising: administering to the subject a therapeutically effective amount of a composition comprising miR-195, miR-195-5p, miR-195-3p or a fragment or variant thereof.

Disclosed herein are methods of treating cognitive impairment in a subject, the methods comprising: administering to the subject a therapeutically effective amount of a composition comprising miR-195, miR-195-5p or miR-195-3p. In some aspects, the methods can further comprise determining the expression level of miR-195, miR-195-5p or miR-195-3p in a sample obtained from the subject before the administration of the composition comprising miR-195, wherein the expression level of the miR-195, miR-195-5p or miR-195-3p is lower when compared to a reference sample. In some aspects, the reference sample can be obtained from a subject that does not have or has not been diagnosed as having cognitive impairment.

The determination that a subject suffers from a cognitive impairment can be made based on the lower expression level of miR-195, miR-195-5p or miR-195-3p compared to a reference value. The determination that a subject suffers from a cognitive impairment can be made based on the lower expression level of miR-195, miR-195-5p or miR-195-3p variants compared to a reference value.

In some aspects, to establish a strict comparison between the relative levels of miRNAs measured from subjects who suffer from a cognitive impairment and the control subjects, it is important to establish a reference value or control value with which to compare the expression levels of the miRNAs of the measured samples. As used herein, the terms reference value and control value are understood as being synonymous. Said control value can be obtained in various ways, for example from previous studies that can be a comparison of cases and controls. In some aspects, the reference value is considered to be the average value of the levels of expression of said miRNA obtained in a statistical study in healthy individuals. In some aspects, miR-195, miR-195-5p or miR-195-3p can be considered to have a value lower than the reference value when the value of its level of expression is one half or less of the average levels of expression in healthy individuals. In some aspects, miR-195, miR-195-5p or miR-195-3p can be considered to have a value lower than the reference value when the value of its level of expression is one half or less of the average levels of expression in age-matched individuals.

In some aspects, the determination of the average level of expression of miR-195, miR-195-5p or miR-195-3p can be achieved using any known method available to a person skilled in the art, for example by calculating the arithmetic mean. In some aspects, the determination of the average level of expression of miR-195, miR-195-5p or miR-195-3p can be achieved by calculating by comparing the averages of the valid duplicates obtained in reference to their average experimental value in a hybridization study with complementary probes that form part of a microarray.

In some aspects, it can also be determined that miR-195, miR-195-5p or miR-195-3p can have a value lower than its reference value when its level of expression is at least four times below the reference value. Other values can also be chosen as reference value, such as for example the value of the 75th percentile of the levels of miR-195, miR-195-5p or miR-195-3p from healthy individuals.

In some aspects, the expression level of miR-195, miR-195-5p or miR-195-3p can be determined by quantitative polymerase chain reaction (PCR).

Disclosed herein are methods of ameliorating one or more symptoms of cognitive impairment in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of reducing synaptojanin 1 (synj1) activity or expression in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of inhibiting synaptojanin 1 (synj1) activity or expression in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are method of increasing amyloid β-protein (Aβ) clearance in a subject, the method comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of reducing traumatic brain injury (TBI)-induced elevation in tau hyper-phosphorylation in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of reducing amyloid plaque burden in a subject. In some aspects, the methods can comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of reducing tau hyper-phosphorylation in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of reducing IL-6 or TNFα release in a subject. Disclosed herein are methods of reducing IL-6 or TNFα release in a subject, the methods comprising administering to the subject a therapeutically effective amount of a composition comprising miR-195-5p or a fragment or variant thereof. Disclosed herein are methods of reducing IL-6 or TNFα release in a subject, the methods comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of decreased phosphorylated tau production in a subject. Disclosed herein are methods of decreasing phosphorylated tau production in a subject, the methods comprising administering to the subject a therapeutically effective amount of a composition comprising miR-195-5p or a fragment or variant thereof. Disclosed herein are methods of decreasing phosphorylated tau production in a subject, the methods comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of treating ischemia induced microglial dysfunction and neuronal injury in a subject. Disclosed herein are methods of treating ischemia induced microglial dysfunction and neuronal injury in a subject, the methods comprising administering to the subject a therapeutically effective amount of a composition comprising miR-195-5p or a fragment or variant thereof. Disclosed herein are methods of treating ischemia induced microglial dysfunction and neuronal injury in a subject, the methods comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of rescuing Alzheimer's disease-related lysosomal defects in a subject. Disclosed herein are methods of rescuing Alzheimer's disease-related lysosomal defects in a subject, the methods comprising administering to the subject a therapeutically effective amount of a composition comprising miR-195-5p or a fragment or variant thereof. Disclosed herein are methods of rescuing Alzheimer's disease-related lysosomal defects in a subject, the methods comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of decreasing expression of pdcd4 and smad7 in a subject, the method comprising administering to the subject a therapeutically effective amount of a composition comprising miR-195-5p or a fragment or variant thereof. Disclosed herein are methods of decreasing expression of pdcd4 and smad7 in a subject, the method comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of decreasing expression of pdcd4 and smad7 and increasing il10a expression in a subject, the method comprising administering to the subject a therapeutically effective amount of a composition comprising miR-195-5p or a fragment or variant thereof. Disclosed herein are methods of decreasing expression of pdcd4 and smad7 and increasing il10a expression in a subject, the method comprising administering to the subject a therapeutically effective amount of a composition comprising miR-195 or a polynucleotide comprising a sequence at least 80% sequence identity to miR-195, miR-195-5p, miR-195-3p or variants and fragments thereof.

Disclosed herein are methods of preventing LPS-induced increased expression of pdcd4 and smad7 in a subject, the method comprising administering to the subject a therapeutically effective amount of a composition comprising miR-195-5p or a fragment or variant thereof. Disclosed herein are methods of preventing LPS-induced increased expression of pdcd4 and smad7 in a subject, the method comprising administering to the subject a therapeutically effective amount of a composition comprising miR-195-5p or a polynucleotide comprising a sequence at least 80% sequence identity to miR-195, miR-195-5p, miR-195-3p or variants and fragments thereof.

Disclosed herein are methods of preventing LPS-induced increased expression of pdcd4 and smad7 and increasing il10a expression in a subject, the method comprising administering to the subject a therapeutically effective amount of a composition comprising miR-195-5p or a fragment or variant thereof, Disclosed herein are methods of preventing LPS-induced increased expression of pdcd4 and smad7 and increasing il10a expression in a subject, the method comprising administering to the subject a therapeutically effective amount of a composition comprising miR-195-5p or a polynucleotide comprising a sequence at least 80% sequence identity to miR-195, miR-195-5p, miR-195-3p or variants and fragments thereof.

Disclosed herein are methods of attenuating lipopolysaccharide-induced proinflammatory cytokine release in a subject, the method comprising administering to the subject a therapeutically effective amount of a composition comprising miR-195-5p or a fragment or variant thereof. Disclosed herein are methods of attenuating Lipopolysaccharide-induced proinflammatory cytokine release in a subject, the method comprising administering to the subject a therapeutically effective amount of a composition comprising miR-195-5p or a polynucleotide comprising a sequence at least 80% sequence identity to miR-195, miR-195-5p, miR-195-3p or variants and fragments thereof.

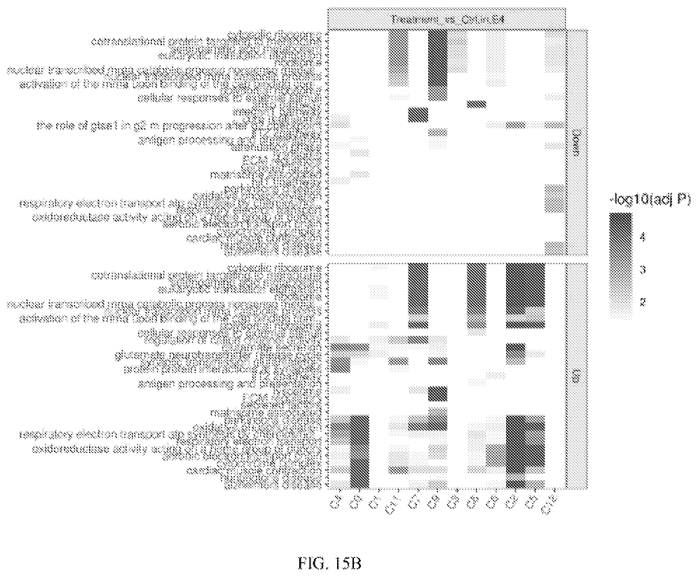

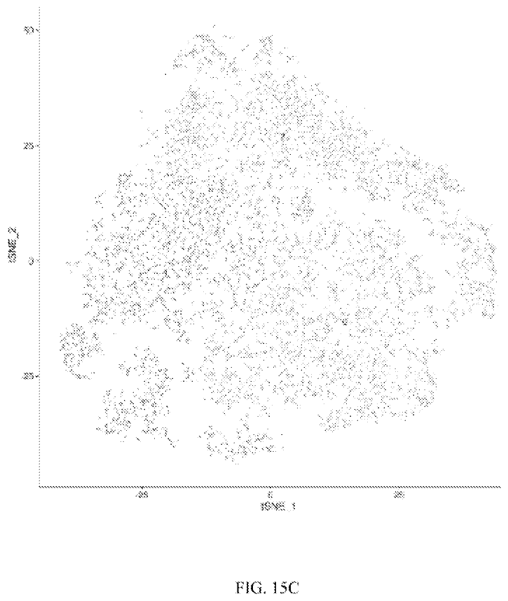

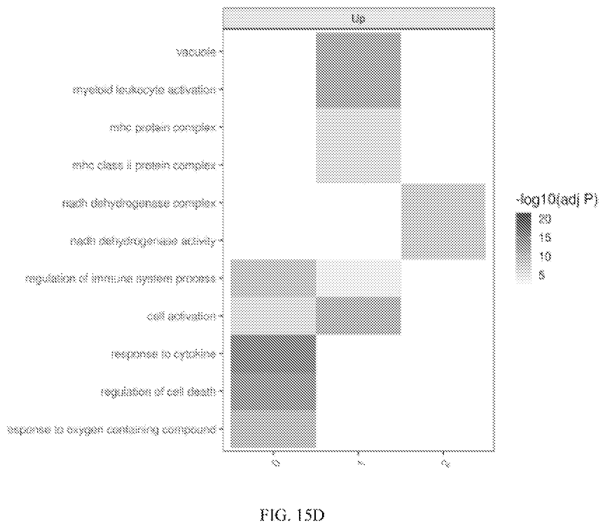

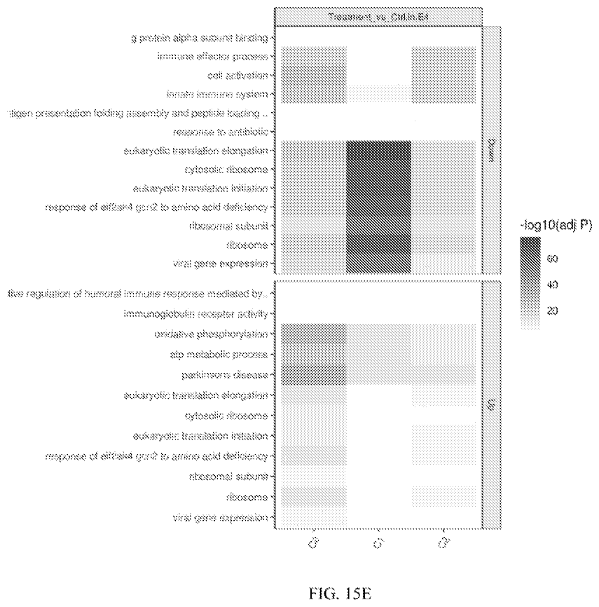

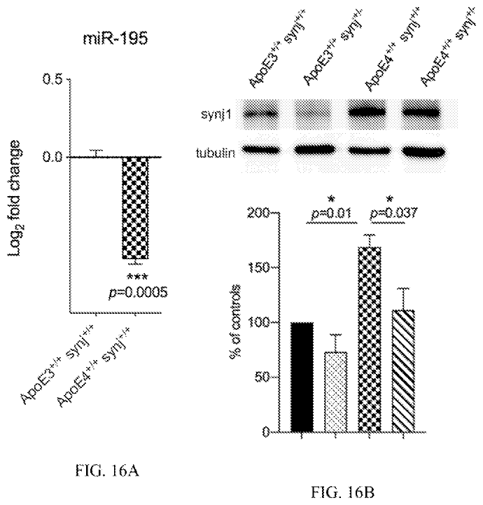

Disclosed herein are methods of down-regulating one or more genes involved innate immune system or effector responses in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof. In some aspects, the one or more genes involved innate immune system or effector responses can be involved in one or more of the pathways in any of FIGS. 15A-E.

Disclosed herein are methods of up-regulating genes involved in mitochondrial and synaptic function within microglial cluster in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof. In some aspects, the one or more genes involved in in mitochondrial and synaptic function within microglial cluster can be involved in one or more of the pathways in any of FIGS. 15A-E.

Disclosed herein are methods of upregulating one or more genes involved in oxidative phosphorylation or ATP metabolic processes in a subject. In some aspects, the methods can comprise administering to the subject a therapeutically effective amount of a composition comprising miR-195 or a polynucleotide comprising a sequence at least 80% sequence identity to miR-195, miR-195-5p, miR-195-3p or variants and fragments thereof. In some aspects, the one or more genes involved in oxidative phosphorylation or ATP metabolic processes can be are involved in one or more of the pathways in any of FIGS. 15A-E.

Disclosed herein are methods of evaluating or monitoring the effectiveness or efficacy of a cognitive impairment treatment in a subject having cognitive impairment or diagnosing cognitive impairment in a subject suspected of having cognitive impairment. The methods can comprise measuring the level of miR-195, miR-195-5p, or miR-195-3p in a sample. In some aspects, the methods can determine the level of miR-195, miR-195-5p, or miR-195-3p before initial treatment with any of the compositions disclosed herein in a sample. In some aspects, the methods can determine the level of miR-195, miR-195-5p, or miR-195-3p any time after a treatment with any of the compositions disclosed herein in a sample. In some aspects, the methods can comprise identifying the cognitive impairment treatment as being effective when the level of miR-195, miR-195-5p, or miR-195-3p is higher after any treatment with any of the compositions disclosed herein than the level of miR-195, miR-195-5p, or miR-195-3p before any of the treatments with any of the compositions disclosed herein. In some aspects, the methods can comprise identifying the cognitive impairment treatment as being not effective when the level of miR-195, miR-195-5p, or miR-195-3p is lower after any treatment with any of the compositions disclosed herein than the level of miR-195, miR-195-5p, or miR-195-3p before any of the treatments with any of the compositions disclosed herein.

In some aspects, the methods include administering a therapeutically effective amount of a composition comprising miR-195, miR-195-5p or miR-195-3p and administering to the subject a therapeutically effective amount of any of the compounds in Table 1 or Table 2 before after or during administration of the composition comprising miR-195, miR-195-5p or miR-195-3p. In some aspects, the methods can also include the administration of a standard of care therapy.

In some aspects, the cognitive impairment can be Alzheimer's disease, mild cognitive impairment, Lewy body dementia (LBD), frontotemporal dementia (FTD), ischemic conditions (e.g., vascular dementia, cerebrovascular disease, and other ischemic processes), mixed dementia, or Down Syndrome. In some aspects, the cognitive impairment can be a traumatic brain injury.

In some aspects, the subject in need of treatment has been diagnosed with a cognitive impairment prior to or before the administering step.

In some aspects, the subject can be a human.

In some aspects, in any of the methods disclosed herein, the miR-195, miR-195-5p or miR-195-3p expression level that is detected can be a sequence comprising at least 90% sequence identity to miR-195, miR-195-5p or miR-195-3p.

In some aspects, in any of the methods disclosed herein the miR-195 that is administered can comprise a sequence comprising at least 80% sequence identity to miR-195.

In some aspects, the miR-195 or the polynucleotide comprising a sequence at least 80% sequence identity to miR-195 can be administered systemically or intrathecally.

In some aspects, the miR-195 or the polynucleotide comprising a sequence at least 80% sequence identity to miR-195 can be administered intranasally.

In some aspects, in any of the methods disclosed herein the miR-195-5p that is administered can comprise a sequence comprising at least 80% sequence identity to miR-195-5p or a fragment or variant thereof.

In some aspects, the miR-195-5p or the polynucleotide comprising a sequence at least 90% sequence identity to miR-195-5p can be administered systemically or intrathecally.

In some aspects, the miR-195-5p or the polynucleotide comprising a sequence at least 90% sequence identity to miR-195-5p can be administered intranasally.

In some aspects, in any of the methods disclosed herein the miR-195-3p that is administered can comprise a sequence comprising at least 80% sequence identity to miR-195-3p or a fragment or variant thereof.

In some aspects, the miR-195-3p or the polynucleotide comprising a sequence at least 80% sequence identity to miR-195-3p can be administered systemically or intrathecally.

In some aspects, the miR-195-3p or the polynucleotide comprising a sequence at least 80% sequence identity to miR-195-3p can be administered intranasally.

In some aspects, the miR-195-5p can be hsa-miR-195-5p comprising the nucleotide sequence set forth in SEQ ID NO: 1. In some aspects, the miR-195-3p can be hsa-miR-195-3p comprising the nucleotide sequence set forth in SEQ ID NO: 2. In some aspects, the miR-195 can be hsa-miR-195 comprising the nucleotide sequence set forth in SEQ ID NO: 5.

In some aspects, the methods described herein can include the administration of miR-195, miR-195-5p, miR-195-3p or variants thereof. In some aspects, the methods described herein can include the administration of miR-195, miR-195-5p, miR-195-3p or fragments thereof.

In some aspects, the sample can be cerebrospinal fluid, brain tissue, serum or plasma.

In some aspects, the sample can have reduced expression of miR-195, miR-195-5p, miR-195-3p when compared to a reference sample before the administration of a composition comprising miR-195, miR-195-5p, miR-195-3p.

In some aspects, a sample can be obtained from a subject (e.g., a cerebrospinal fluid sample from the subject) and the level of expression of miR-195, miR-195-5p, miR-195-3p in the sample can be compared to a reference sample. The reference sample or control sample or reference cell can be from a subject that does not have a cognitive impairment and can be generally of the same type as the cell or sample. In some aspects, the reference sample can be a normal sample or cell from the subject from whom the tissue or a cell that is suspected of having a cognitive impairment is obtained. The reference sample can be, but is not required to be from the same subject. In some aspects, the reference sample can be provided from a different subject who is established or known to not have the specific cognitive impairment being compared. In some aspects, the reference sample can be sex-matched, age-matched and/or race-matched to the subject whose sample is being compared.

In some aspects, the reference sample can be the mean expression level (as a measure of the number of samples or cells used) obtained from the expression levels of miR-195, miR-195-5p, miR-195-3p from a number of individuals, wherein the same histological type as the cell being assayed is used to measure the miR-195, miR-195-5p, miR-195-3p expression level, and wherein all of the individuals used to obtain the mean expression or miR-195, miR-195-5p, miR-195-3p expression level do not have a cognitive impairment. In this case, the individuals can be from different age groups, races and sexes. Alternatively, the individuals may be from the same age groups, race and/or sex.

As used herein, the term “expression,” when used in the context of determining or detecting the expression or expression level of one or more genes, can refer to determining or detecting transcription of the gene (i.e., determining mRNA levels) and/or determining or detecting translation of the gene (e.g., determining or detecting the protein produced). To determine the expression level of a gene means to determine whether or not a gene is expressed, and if expressed, to what relative degree. The expression level of one or more genes disclosed herein can be determined directly (e.g., immunoassays, mass spectrometry) or indirectly (e.g., determining the mRNA expression of a protein or peptide). Examples of mass spectrometry include ionization sources such as EI, CI, MALDI, ESI, and analysis such as Quad, ion trap, TOF, FT or combinations thereof, spectrometry, isotope ratio mass spectrometry (IRMS), thermal ionization mass spectrometry (TIMS), spark source mass spectrometry, Multiple Reaction Monitoring (MRM) or SRM. Any of these techniques can be carried out in combination with prefractionation or enrichment methods. Examples of immunoassays include immunoblots, Western blots, Enzyme linked Immunosorbant Assay (ELISA), Enzyme immunoassay (EIA), radioimmune assay. Immunoassay methods use antibodies for detection and determination of levels of an antigen are known in the art. The antibody can be immobilized on a solid support such as a stick, plate, bead, microbead or array.

Expression levels of one or more of the genes described herein can be also be determined indirectly by determining the mRNA expression for the one or more genes in a tissue sample. RNA expression methods include but are not limited to extraction of cellular mRNA and Northern blotting using labeled probes that hybridize to transcripts encoding all or part of the gene, amplification of mRNA using gene-specific primers, polymerase chain reaction (PCR), and reverse transcriptase-polymerase chain reaction (RT-PCR), followed by quantitative detection of the gene product by a variety of methods; extraction of RNA from cells, followed by labeling, and then used to probe cDNA or oligonucleotides encoding the gene, in situ hybridization; RNA-sequencing; and detection of a reporter gene.

Methods to measure protein expression levels include but are not limited to Western blot, immunoblot, ELISA, radioimmunoassay, immunoprecipitation, surface plasmon resonance, chemiluminescence, fluorescent polarization, phosphorescence, immunohistochemical analysis, microcytometry, microarray, microscopy, fluorescence activated cell sorting (FACS), and flow cytometry. The method can also include specific protein property-based assays based including but not limited to enzymatic activity or interaction with other protein partners. Binding assays can also be used, and are well known in the art. For instance, a BIAcore machine can be used to determine the binding constant of a complex between two proteins. Other suitable assays for determining or detecting the binding of one protein to another include, immunoassays, such as ELISA and radioimmunoassays. Determining binding by monitoring the change in the spectroscopic can be used or optical properties of the proteins can be determined via fluorescence, UV absorption, circular dichroism, or nuclear magnetic resonance (NMR). Alternatively, immunoassays using specific antibody can be used to detect the expression on of a particular protein on a tumor cell.

As used herein, the term “reference,” “reference expression,” “reference sample,” “reference value,” “control,” “control sample” and the like, when used in the context of a sample or expression level of one or more genes or proteins or microRNAs refers to a reference standard wherein the reference is expressed at a constant level among different (i.e., not the same tissue, but multiple tissues) tissues, and is unaffected by the experimental conditions, and is indicative of the level in a sample of a predetermined disease status (e.g., not suffering from cognitive impairment). The reference value can be a predetermined standard value or a range of predetermined standard values, representing no illness, or a predetermined type or severity of illness.

Reference expression can be the level of the one or more genes or proteins or microRNAs described herein in a reference sample from a subject, or a pool of subjects, not suffering from cognitive impairment or from a predetermined severity or type of cognitive impairment. In some aspects, the reference value can be the level of one or more genes or proteins or microRNAs disclosed herein in the tissue of a subject, or subjects, wherein the subject or subjects is not suffering from cognitive impairment.

Determining the expression level of one or more genes or proteins or microRNAs disclosed herein can include determining whether the gene or proteins or microRNAs is upregulated or increased as compared to a control or reference sample, downregulated or decreased (e.g., low) compared to a control or reference sample, or unchanged compared to a control or reference sample. As used herein, the terms, “upregulated: and “increased expression level” or “increased level of expression” refers to a sequence corresponding to one or more genes or proteins or microRNAs disclosed herein that is expressed wherein the measure of the quantity of the sequence exhibits an increased level of expression (e.g., high) when compared to a reference sample or “normal” control. For example, the terms, “upregulated” and “increased expression level” or “increased level of expression” refers to a sequence corresponding to one or more genes disclosed herein that is expressed wherein the measure of the quantity of the sequence exhibits an increased level of expression of one or more of protein(s) and/or mRNA when compared to the expression of the same mRNA(s) from a reference sample or “normal” control. An “increased expression level” refers to an increase in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more. As used herein, the terms “downregulated,” “decreased level of expression,” or “decreased expression level” refers to a sequence corresponding to one or more genes or proteins or microRNAs disclosed herein that is expressed wherein the measure of the quantity of the sequence exhibits a decreased level of expression when compared to a reference sample or “normal” control. For example, the terms “downregulated,” “decreased level of expression,” or “decreased expression level” refers to a sequence corresponding to one or more genes disclosed herein that is expressed wherein the measure of the quantity of the sequence exhibits a decreased level of expression of one or more protein(s) and/or mRNA when compared to the expression of the same mRNA(s) from a reference sample or “normal” control. A “decreased level of expression” refers to a decrease in expression of at least 1%, 2%, 3%, 4%, 5%, 6%, 7%, 8%, 9%, 10% or more, for example, 20%, 30%, 40%, or 50%, 60%, 70%, 80%, 90% or more, or greater than 1-fold, up to 2-fold, 3-fold, 4-fold, 5-fold, 10-fold, 50-fold, 100-fold or more.

In some aspects, the efficacy of the cognitive impairment treatments can be determined. The levels of miR-195, miR-195-5p, or miR-195-3p can serve as a biomarker for the efficacy of the cognitive impairment treatment. For example, the levels of miR-195-5p in the post-treatment cells can be higher than the levels of miR-195-5p in the pre-treatment cells, then the treatment can be deemed to be effective. In some aspects, the methods can further comprise measuring the levels of miR-195, miR-195-5p, or miR-195-3p after administering to a subject a composition comprising miR-195-5p to the subject.

In some aspects, a relative level of synaptojanin 1 can be measured. High or increased levels of synaptojanin 1 correspond to low levels of miR-195, miR-195-5p, miR-195-3p.

In some aspects, the efficacy of the treatments for cognitive impairment can be determined. The levels of miR-195, miR-195-5p, miR-195-3p can serve as a biomarker for the efficacy of the treatment for cognitive impairment. For example, the levels of miR-195, miR-195-5p, miR-195-3p in a sample can be higher than the levels of miR-195, miR-195-5p, miR-195-3p, respectively in the pre-treatment sample, then the treatment can be deemed to be effective.

In some aspects, the miR-195 can be hsa-miR-195. In some aspects, the miR-195 can comprise the nucleotide sequence UAGCAGCACAGAAAUAUUGGC (SEQ ID NO: 5). In some aspects, the composition can comprise a sequence derived from miR-195. In some aspects, the miR-195-5p can be hsa-miR-195-5p. In some aspects, the miR-195-5p can comprise the nucleotide sequence UAGCAGCACAGAAAUAUUGGC (SEQ ID NO: 1). In some aspects, the miR-195-3p can be hsa-miR-195-3p. In some aspects, the miR-195-3p can comprise the nucleotide sequence CCAAUAUUGGCUGUGCUGCUCC (SEQ ID NO: 2).

Disclosed herein, are methods of suppressing expression of the synaptojanin 1 gene in a cell. In some aspects, the methods can comprise contacting a cell with miR-195, a fragment thereof or a variant thereof. In some aspects, the methods can comprise contacting a cell with miR-195-5p, a fragment thereof or a variant thereof. In some aspects, the methods can comprise contacting a cell with miR-195-3p, a fragment thereof or a variant thereof. In some aspects, the synaptojanin 1 gene encodes phosphoinositide phosphatase that regulates levels of membrane phosphatidylinositol-4,5-bisphosphate; thereby suppressing the expression of the synaptojanin 1 gene in cell when compared to a reference sample. In some aspects, the cell can be a brain cell. The methods can include contacting a cell with a therapeutically effective amount of miR-195, miR-195-5p or miR-195-3p.

Contacting a cell with a miR-195, a fragment or a variant thereof, miR-195-5p, or a fragment or variant thereof, miR-195-3p, a fragment or a variant thereof or molecule capable of stimulating or enhancing the expression or activity of a miR-195, miR-195-5p or miR-195-3p can be achieved by any method known in the art. In some aspects, contacting the cell and the miR-195, miR-195-5p or miR-195-3p occur in vivo. The miR-195, miR-195-5p or miR-195-3p or molecule capable of stimulating or enhancing the expression or activity of a miR-195, miR-195-5p or miR-195-3p or molecule may be contacted with the cell directly, for example, applied directly to a cell that is associated one or more symptoms of cognitive impairment or alternatively can be combined with the cell indirectly, e.g. by injecting the molecule into the bloodstream of a subject, which then carries the molecule to the cell that is associated one or more symptoms of cognitive impairment. Further, a sample can be removed from a subject and combined with miR-195, miR-195-5p or miR-195-3p or molecule capable of stimulating or enhancing the expression or activity of a miR-195, miR-195-5p or miR-195-3p in vitro prior to returning at least a portion of the sample back to the subject. For example, the sample can be a blood, serum, plasma or cerebrospinal fluid sample which can be removed from a subject and combined with the miR-195, miR-195-5p or miR-195-3p prior to injecting at least a portion of the blood, serum, plasma or cerebrospinal fluid back into the subject.

The compositions described herein can be formulated to include a therapeutically effective amount of miR-195, a fragment or a variant thereof, miR-195-5p, or a fragment or variant thereof, or miR-195-3p, a fragment or a variant thereof described herein. Therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to a type of cognitive impairment.

The compositions described herein can be formulation in a variety of combinations. The particular combination of miR-195, a fragment or a variant thereof, miR-195-5p, or a fragment or variant thereof, or miR-195-3p, a fragment or a variant thereof with any of the compounds in Table 1 and Table 2 can vary according to many factors, for example, the particular the type and severity of the cognitive impairment.

The compositions described herein can be administered to the subject (e.g., a human patient) in an amount sufficient to delay, reduce, or preferably prevent the onset of clinical disease. Accordingly, in some aspects, the patient can be a human patient. In some aspects, the human subject or patient can be a child or an adult. In therapeutic applications, compositions are administered to a subject (e.g., a human patient) already with or diagnosed with cognitive impairment in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the cognitive impairment is delayed, hindered, or prevented, or the cognitive impairment or a symptom of the cognitive impairment is ameliorated. One or more of the symptoms can be less severe. Recovery can be accelerated in an individual who has been treated.

The compositions described herein can be formulated to include a therapeutically effective amount of miR-195, a fragment or a variant thereof, miR-195-5p, or a fragment or variant thereof, or miR-195-3p, a fragment or a variant thereof alone or in combination with one or more of the compounds disclosed in Table 1 and Table 2. In some aspects, miR-195, a fragment or a variant thereof, miR-195-5p, or a fragment or variant thereof, or miR-195-3p, a fragment or a variant thereof can be contained within a pharmaceutical formulation. In some aspects, the pharmaceutical formulation can be a unit dosage formulation.

The therapeutically effective amount or dosage of the miR-195, a fragment or a variant thereof, miR-195-5p, or a fragment or variant thereof, or miR-195-3p, a fragment or a variant thereof and any of the compounds described in Table 1 and Table 2 used in the methods as disclosed herein applied to mammals (e.g., humans) can be determined by one of ordinary skill in the art with consideration of individual differences in age, weight, sex, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cognitive impairment symptoms), the age and physical characteristics of the subject and other considerations known to those of ordinary skill in the art. Dosages can be established using clinical approaches known to one of ordinary skill in the art.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Compositions comprising miR-195, miR-195-5p or miR-195-3p (including fragments and variants thereof) can be administered to a subject in a dose or doses of about or of at least about 0.5, 1, 5, 10, 15, 20, 25, 30, 35, 40, 45, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1500, 2000, 2500, 3000 g or mg, or any range between 0.5 μg or mg and 3000 μg or mg. The amount specified can be the amount administered as the average daily, average weekly, or average monthly dose, or it may be expressed in terms of mg/kg, where kg refers to the weight of the patient and the mg is specified above. In other embodiments, the amount specified is any number discussed above but expressed as mg/m² (with respect to tumor size or patient surface area). A clinician can readily determine the effective amount of a miR-195, miR-195-5p or miR-195-3p—i.e. the amount of miR-195, miR-195-5p, miR-195-3p, or fragments or variant thereof needed to increase endogenous miR-195, miR-195-5p or miR-195-3p expression levels, decrease synaptojanin 1 activity or expression, increasing amyloid β-protein clearance, decrease or reduce amyloid plaque burden, decrease or reduce tau hyper-phosphorylation, or rescue Alzheimer's disease-related lysosomal defects in a subject or in a cell, by taking into account factors, such as the size and weight of the subject; the extent of disease penetration; the age, health and sex of the subject; the route of administration; and whether the administration is regional or systemic.

In some aspects, the dosages of miR-195, miR-195-5p, miR-195-3p (including fragments and variants thereof) can be less when combined with one or more of the compounds disclosed in Table 1 and Table 2.

The total effective amount of the compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The compositions described herein can be administered in conjunction with other therapeutic modalities to a subject in need of therapy. For example, in the methods disclosed herein, the compositions described herein can be administered in conjunction with other therapeutic modalities to a subject in need of therapy. The miR-195, miR-195-5p, miR-195-3p, or fragments or variants thereof can be given prior to, simultaneously with or after treatment with other agents or regimes. In some aspects, miR-195, miR-195-5p, miR-195-3p, or fragments or variants thereof can be given prior to, simultaneously or during, or after administration of one or more of the compounds described in Table 1 and Table 2 or one or more drugs that increase miR-195 expression levels or a combination of both. For example, miR-195, or a variant thereof alone or with any of the compounds disclosed in Table 1 and Table 2 can be administered in conjunction with standard therapies used to treat cognitive impairment. In some aspects, miR-195, miR-195-5p, miR-195-3p, or fragments or variants thereof can be administered or used together with one or more of the compounds described in Table 1 and Table 2 or a combination thereof.

In some aspects, miR-195, miR-195-5p, miR-195-3p, or fragments or variants thereof and one or more of the compounds disclosed in Table 1 and Table 2 can be co-formulated. The compositions described herein can be formulated to include a therapeutically effective amount of miR-195, miR-195-5p, or miR-195-3p in combination with one or more of the compounds disclosed in Table 1 and Table 2. In some aspects, miR-195, miR-195-5p, miR-195-3p, or fragments or variants thereof and one or more compounds disclosed in Table 1 and Table 2 can be co-formulated inside a nanoparticle. In some aspects, miR-195, miR-195-5p, miR-195-3p, or fragments or variants thereof can be contained within a pharmaceutical formulation. In some aspects, the pharmaceutical formulation can be a unit dosage formulation.

In some aspects, the methods of treatment disclosed herein can also include the administration of a therapeutically effective amount of immunotherapy, stem cell transplantation or a combination thereof. The combination therapies disclosed can be administered as one or more pharmaceutical compositions and, if separately, can be administered simultaneously or sequentially in any order.

In some aspects, any of the compositions disclosed herein can be administered with one or more immunotherapeutic or immune modulating agents. As used herein, the terms “immunomodulatory” and “immune modulating agents” refer to a component (e.g., a protein, peptide, pharmacological and/or immunological agent) that modifies (e.g., potentiates) the immune system response toward a desired immune system response. An immunomodulator can also be an adjuvant. The immunomodulator can be a therapeutic agent that specifically or nonspecifically augments an immune system response. Examples of immunomodulators or immune modulating agents include but are not limited to cytokines, interleukins, chemokines or any protein, peptide, pharmacological or immunological agent that provides an increase in an immune system response. Examples of immunotherapeutic agents can include but are not limited antibody therapy, cytokine therapy, and combination immunotherapy. In some aspects, the immune modulating agent can be an anti-amyloid antibody or anti-tau antibody. The compositions described herein can be a combination therapy for a disease.

The miR-195, miR-195-5p, miR-195-3p, or fragments or variants thereof can be administered as “combination” therapy. It is to be understood that, for example, miR-195, miR-195-5p, miR-195-3p, or fragments or variants thereof can be provided to the subject in need, either prior to administration of any of the compounds disclosed in Table 1 and Table 2 or any combination thereof, concomitant with administration of said any of the compounds disclosed in Table 1 and Table 2 or any combination thereof (co-administration) or shortly thereafter.

### Pharmaceutical Compositions

As disclosed herein, are pharmaceutical compositions, comprising miR-195 and a pharmaceutical acceptable carrier described herein. In some aspects, miR-195 can be formulated for oral or parental administration. As disclosed herein, are pharmaceutical compositions, comprising miR-195-5p and a pharmaceutical acceptable carrier described herein. In some aspects, miR-195-3p can be formulated for oral or parental administration. In some aspects, the parental administration is intravenous, subcutaneous, intranasal, intramuscular or direct injection. The compositions can be formulated for administration by any of a variety of routes of administration, and can include one or more physiologically acceptable excipients, which can vary depending on the route of administration. As used herein, the term “excipient” means any compound or substance, including those that can also be referred to as “carriers” or “diluents.” Preparing pharmaceutical and physiologically acceptable compositions is considered routine in the art, and thus, one of ordinary skill in the art can consult numerous authorities for guidance if needed.

The compositions can be administered directly to a subject. Generally, the compositions can be suspended in a pharmaceutically acceptable carrier (e.g., physiological saline or a buffered saline solution) to facilitate their delivery. Encapsulation of the compositions in a suitable delivery vehicle (e.g., polymeric microparticles or implantable devices) may increase the efficiency of delivery.

The compositions can be formulated in various ways for parenteral or nonparenteral administration. Where suitable, oral formulations can take the form of tablets, pills, capsules, or powders, which may be enterically coated or otherwise protected. Sustained release formulations, suspensions, elixirs, aerosols, and the like can also be used.

Pharmaceutically acceptable carriers and excipients can be incorporated (e.g., water, saline, aqueous dextrose, and glycols, oils (including those of petroleum, animal, vegetable or synthetic origin), starch, cellulose, talc, glucose, lactose, sucrose, gelatin, malt, rice, flour, chalk, silica gel, magnesium stearate, sodium stearate, glycerol monosterate, sodium chloride, dried skim milk, glycerol, propylene glycol, ethanol, and the like). The compositions may be subjected to conventional pharmaceutical expedients such as sterilization and may contain conventional pharmaceutical additives such as preservatives, stabilizing agents, wetting or emulsifying agents, salts for adjusting osmotic pressure, buffers, and the like. Suitable pharmaceutical carriers and their formulations are described in “Remington's Pharmaceutical Sciences” by E.W. Martin, which is herein incorporated by reference. Such compositions will, in any event, contain an effective amount of the compositions together with a suitable amount of carrier so as to prepare the proper dosage form for proper administration to the patient.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used. Thus, compositions can be prepared for parenteral administration that includes miR-195 dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like).

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules.

In some aspects, miR-195, miR-195-5p, miR-195-3p or a polynucleotide comprising a sequence at least 80% sequence identity to miR-195, miR-195-5p, or miR-195-3p, respectively can be administered systemically. In some aspect, miR-195, miR-195-5p, miR-195-3p or a polynucleotide comprising a sequence at least 80% sequence identity to miR-195, miR-195-5p, or miR-195-3p, respectively can be administered intravenously. In some aspect, miR-195, miR-195-5p, miR-195-3p or a polynucleotide comprising a sequence at least 80% sequence identity to miR-195, miR-195-5p, or miR-195-3p, respectively can be administered intrathecally. In some aspect, miR-195, miR-195-5p, miR-195-3p or a polynucleotide comprising a sequence at least 80% sequence identity to miR-195, miR-195-5p, or miR-195-3p, respectively can be administered intranasally. In some aspects, the pharmaceutical composition can be formulated for systemic, intravenous, intranasal or intrathecal administration. In some aspects, the composition can be formulated in a lipid emulsion. In some aspects, the miR-195, miR-195-5p, miR-195-3p or a polynucleotide comprising a sequence at least 80% sequence identity to miR-195, miR-195-5p, or miR-195-3p, respectively can be formulated for delivery in a lipid emulsion, a liposome, a nanoparticle, an exosome, or in a viral vector. The liposome can be a unilamellar, multilamellar, or multivesicular liposome. A wide variety of liposomes and exosomes can be used. For example, in some aspects, a silicone nanoparticle can be used to deliver a miR-195, miR-195-5p, or miR-195-3p to a cell. In some aspects, a nanovector can be used to deliver a miR-195, miR-195-5p, miR-195-3p or a polynucleotide comprising a sequence at least 80% sequence identity to miR-195, miR-195-5p, or miR-195-3p, respectively to a subject. In some aspects, compositions comprising miR-195, miR-195-5p, miR-195-3p or a polynucleotide comprising a sequence at least 80% sequence identity to miR-195, miR-195-5p, or miR-195-3p, respectively can be administered into the cerebrospinal fluid by injection into the subarachnoid space of the spinal cord to bypass the blood-brain barrier.

In some aspects, miR-195, miR-195-5p, miR-195-3p or a polynucleotide comprising a sequence at least 80% sequence identity to miR-195, miR-195-5p, or miR-195-3p, respectively can be encoded by a nucleic acid. The nucleic acid can be transfected into one or more cells. The transfection can comprise electroporation or incubation with a viral vector. In some aspects, the nucleic acid can be located in a vector. In some aspects, the vector can be plasmid, cosmid, phagemid or a viral vector. In some aspects, the vector can comprise a lipid, lipid emulsion, liposome, nanoparticle or exosomes. In some aspects, nucleic acid can be comprised in a lipid, lipid emulsion, liposome, nanoparticle or exosome. In some aspects, the viral vector can be an adenovirus, an adeno-associated virus, a lentivirus or a herpes simplex virus. In some aspects, the vector can comprise a lipid, lipid emulsion, liposome, nanoparticle or exosomes.

Nanoparticles. The compositions described herein can comprise one or more nanoparticles. The nanoparticle compositions disclosed herein can be used to enhance delivery of conjugated or entrapped miR-195, miR-195-5p, miR-195-3p or a polynucleotide comprising a sequence at least 90% sequence identity to miR-195, miR-195-5p, or miR-195-3p, respectively across the blood brain barrier. Examples of nanoparticles (used interchangeably with the term “nanocarrier”) can be found, for example, in U.S. Patent Publication No. 2010-0233251. Examples of nanocarriers include, but are not limited to nanocarriers comprising one or more polymers. In some aspects, the one or more polymers can be a water soluble, non-adhesive polymer. In some aspects, the polymer can be polyethylene glycol (PEG) or polyethylene oxide (PEO). In some aspects, the polymer can be a polyalkylene glycol or polyalkylene oxide. In some aspects, the one or more polymers can be a biodegradable polymer. In some aspects, the one or more polymers can be a biocompatible polymer that can be a conjugate of a water soluble, non-adhesive polymer and a biodegradable polymer. In some aspects, the biodegradable polymer can be polylactic acid (PLA), poly(glycolic acid) (PGA), or poly(lactic acid/glycolic acid) (PLGA). In some aspects, the nanocarrier can be composed of PEG-PLGA polymers.

In some aspects, the nanocarrier can be formed by self-assembly. Self-assembly refers to the process of the formation of a nanocarrier using components that will orient themselves in a predictable manner forming nanocarriers predictably and reproducibly. In some aspects, the nanocarriers can be formed using amphiphillic biomaterials which orient themselves with respect to one another to form nanocarriers of predictable dimension, constituents, and placement of constituents. In some aspects, the nanocarrier can be a microparticle, nanoparticle, or picoparticle. In some aspects, the microparticle, nanoparticle, or picoparticle can be self-assembled.

In some aspects, the nanocarrier can have a positive zeta potential. In some aspects, the nanocarrier can have a net positive charge at neutral pH. In some aspects, the nanocarrier can comprise one or more amine moieties at its surface. In some aspects, the amine moiety can be a primary, secondary, tertiary, or quaternary amine. In some aspects, the amine moiety can be an aliphatic amine. In some aspects, the nanocarrier can comprise an amine-containing polymer. In some aspects, the nanocarrier can comprise an amine-containing lipid.

In some aspects, the nanocarrier can comprise a protein or a peptide that can be positively charged at neutral pH. In some aspects, the nanocarrier can be a latex particle. In some aspects, the nanocarrier with the one or more amine moieties on its surface can have a net positive charge at neutral pH.

Nanoparticles can aid the delivery of the miR-195, miR-195-5p, miR-195-3p or a polynucleotide comprising a sequence at least 80% sequence identity to miR-195, miR-195-5p, miR-195-3p, respectively. Delivery can be to a particular site of interest, e.g., the brain cells, frontotemporal cortical cells. In some aspects, the nanoparticle can create a timed release of the miR-195, miR-195-5p, miR-195-3p or a polynucleotide comprising a sequence at least 80% sequence identity to miR-195, miR-195-5p, or miR-195-3p, respectively to enhance and/or extend the therapeutic response. In some aspects, the nanoparticle can be associated with the miR-195, miR-195-5p, miR-195-3p or a polynucleotide comprising a sequence at least 80% sequence identity to miR-195, miR-195-5p, or miR-195-3p, respectively. The association can be, for example, wherein the nanoparticle can be coupled or conjugated with the miR-195, miR-195-5p, miR-195-3p or a polynucleotide comprising a sequence at least 80% sequence identity to miR-195, miR-195-5p, or miR-195-3p, respectively. The terms “coupled” and “conjugated” are meant that there is a chemical linkage between the nanoparticle and the miR-195, miR-195-5p, miR-195-3p or a polynucleotide comprising a sequence at least 80% sequence identity to miR-195, miR-195-5p, or miR-195-3p, respectively. In some aspects, the miR-195, miR-195-5p, miR-195-3p or a polynucleotide comprising a sequence at least 80% sequence identity to miR-195, miR-195-5p, or miR-195-3p, respectively can be entrapped or encapsulated within the nanoparticle. In some aspects, the miR-195, miR-195-5p, miR-195-3p or a polynucleotide comprising a sequence at least 80% sequence identity to miR-195, miR-195-5p, miR-195-3p, respectively, can be entrapped within the nanoparticle by a water/oil/water emulsion method. In some aspects, the nanoparticle can be poly(lactide co-glycolide) (PLGA). Depending on the ratio of lactide to glycolide used for the polymerization, different forms of PLGA can be obtained and utilized. These forms are typically identified in regard to the monomers' ratio used (e.g., PLGA 75:25 identifies a copolymer whose composition can be 75% lactic acid and 25% glycolic acid). Different ratios can be used in this invention, e.g., 90:10, 80:20, 70:30, 60:40, 50:50, 40:60, 30:70, 20:80, 10:90, and numbers above and in between these ratios. Additional examples of suitable nanoparticles include chitosin, calcium phosphate, lipids of various bacteria like E. coil, mycobacteria, leptospira and mixtures thereof. In one example, the composition can be derived mixing about 180 mg of PLGA to about 5 mg of miR-195, miR-195-5p, or miR-195-3p (or about 36 mg PLGA to 1 mg miR-195, miR-195-5p, or miR-195-3p, respectively). The entrapment (encapsulation) efficiency of miR-195, miR-195-5p, or miR-195-3p can vary. In some aspects, the nanoparticle can be 50-55% entrapped/encapsulated, calculated based on amount of total miR-195, miR-195-5p, miR-195-3p or a polynucleotide comprising a sequence at least 90% sequence identity to miR-195, miR-195-5p, or miR-195-3p, respectively used in the entrapment. Entrapped miR-195, miR-195-5p, miR-195-3p or a polynucleotide comprising a sequence at least 90% sequence identity to miR-195, miR-195-5p, or miR-195-3p, respectively can be administered as mixtures of entrapped/encapsulated and unentrapped/unencapsulated miR-195, miR-195-5p, miR-195-3p or a polynucleotide comprising a sequence at least 80% sequence identity to miR-195, miR-195-5p, or miR-195-3p, respectively, or the entrapped/encapsulated miR-195, miR-195-5p, miR-195-3p or a polynucleotide comprising a sequence at least 80% sequence identity to miR-195, miR-195-5p, or miR-195-3p, respectively can be further purified.

In some aspects, miR-195, miR-195-5p, miR-195-3p or a polynucleotide comprising a sequence at least 80% sequence identity to miR-195, miR-195-5p, or miR-195-3p, respectively can be conjugated to copolymer. Traditional copolymers have been used in numerous laboratories worldwide and also in several clinical trials. (See U.S. Pat. No. 5,037,883, which is hereby incorporated by reference in its entirety). For example, N-(2-hydroxypropyl)methacrylamide) (HPMA) copolymers are: (1) biocompatible and have a well-established safety profile; (2) water-soluble and have favorable pharmacokinetics when compared to low molecular weight (free, non-attached) drugs; and (3) possess excellent chemistry flexibility (i.e., monomers containing different side chains can be easily synthesized and incorporated into their structure). However, HPMA polymers are not degradable and the molecular weight of HPMA polymers should be kept below the renal threshold to sustain biocompatibility. This limits the intravascular half-life and accumulation of HPMA polymers in solid tumor via the EPR (enhanced permeability and retention) effect.

A backbone degradable HPMA copolymer carrier can be used to overcome limitations associated with HPMA. The copolymer carrier can contain enzymatically degradable sequences (i.e., by Cathepsin B, matrix matalloproteinases, etc.) in the main chain (i.e., the polymer backbone) and enzymatically degradable side chains (i.e., for drug release). (See, e.g., U.S. patent application Ser. No. 13/583,270, which is hereby incorporated by reference in its entirety). Upon reaching the lysosomal compartment of cells, the drug can be released and concomitantly the polymer carrier can be degraded into molecules that are below the renal threshold and can be eliminated from the subject. Thus, diblock or multiblock biodegradable copolymers with increased molecular weight can be produced. This can further enhance the blood circulation time of the copolymer-miR-195, -miR-195-5p, or -miR-195-3p therapeutic conjugate disclosed herein. Furthermore, U.S. Pat. No. 4,062,831 describes a range of water-soluble polymers and U.S. Pat. No. 5,037,883 describes a variety of peptide sequences, both of which are hereby incorporated by reference in their entireties.

In some instances, the miR-195, miR-195-5p, miR-195-3p or a polynucleotide comprising a sequence at least 90% sequence identity to miR-195, miR-195-5p, or miR-195-3p, respectively can be conjugated to HPMA copolymers administered in the disclosed methods can comprise 2, 3, 4, 5, 6, 7, 8, 9, or 10 HPMA copolymers. In some instances, each HPMA copolymer can be connected via enzymatically degradable peptides.

In some aspects, the miR-195, miR-195-5p, miR-195-3p or a polynucleotide comprising a sequence at least 80% sequence identity to miR-195, miR-195-5p, or miR-195-3p, respectively can be conjugated to HPMA copolymers administered in the disclosed methods can also comprise a linker. In some aspects, the linker can be a peptide linker.

Vectors can include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In some aspects, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

### Articles of Manufacture

The composition described herein can be packaged in a suitable container labeled, for example, for use as a therapy to treat cognitive impairment or any of the methods disclosed herein. Accordingly, packaged products (e.g., sterile containers containing the composition described herein and packaged for storage, shipment, or sale at concentrated or ready-to-use concentrations) and kits, including at least miR-195, miR-195-5p, miR-195-3p or a polynucleotide comprising a sequence at least 80% sequence identity to miR-195, miR-195-5p, or miR-195-3p, respectively as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, syringes, buffers or other control reagents for treating or monitoring the condition for which prophylaxis or treatment is required. The product may also include a legend (e.g., a printed label or insert or other medium describing the product's use (e.g., an audio- or videotape)). The legend can be associated with the container (e.g., affixed to the container) and can describe the manner in which the compound therein should be administered (e.g., the frequency and route of administration), indications therefor, and other uses. The compounds can be ready for administration (e.g., present in dose-appropriate units), and may include a pharmaceutically acceptable adjuvant, carrier or other diluent. Alternatively, the compounds can be provided in a concentrated form with a diluent and instructions for dilution.

In some aspects, the kits can include one or more of miR-195, miR-195-5p, or miR-195-3p or molecules derived from miR-195, miR-195-5p, or miR-195-3p or a polynucleotide comprising a sequence at least 80% sequence identity to miR-195, miR-195-5p, or miR-195-3p, respectively; expression vectors comprising nucleic acid sequences encoding miR-195, miR-195-5p, or miR-195-3p or one or more molecules derived from miR-195, miR-195-5p, or miR-195-3p or a polynucleotide comprising a sequence at least 80% sequence identity to miR-195, miR-195-5p, or miR-195-3p, respectively; reagents for preparing samples from cerebrospinal fluid samples. The kit can include one or more pharmaceutically acceptable carriers. In addition, devices or materials for administration of the miR-195, miR-195-5p, miR-195-3p or a polynucleotide comprising a sequence at least 80% sequence identity to miR-195, miR-195-5p, or miR-195-3p, respectively (e.g., syringes (pre-filled with miR-195, miR-195-5p, miR-195-3p or a polynucleotide comprising a sequence at least 80% sequence identity to miR-195, miR-195-5p, or miR-195-3p, respectively), needles, liposomes, etc.) can also be included.

### EXAMPLES

### Example 1: MicroRNA-195-5p Rescues ApoE4-Induced Cognitive Deficits and Lysosomal Defects in Alzheimer's Disease Pathogenesis

Abstract. Described herein is a link between apolipoprotein E4 (ApoE4)-specific changes in brain phosphoinositol biphosphate (PIP₂) homeostasis to the susceptibility of developing Alzheimer's Disease (AD). miR-195-5p was identified as atop micro-RNA candidate involved in the ApoE/PIP₂ pathway using miRNA profiles in human ROSMAP datasets and mouse microarray studies. Further validation studies have demonstrated that levels of miR-195-5p are significantly lower in human brain tissue of ApoE4^{+/-} patients with clinical diagnosis of mild cognitive impairment (MCI) or early AD when compared to ApoE4^{-/-} subjects. In addition, brain miR-195-5p levels are reduced along with disease progression from normal aging to early AD, and cerebrospinal fluid (CSF) miR-195-5p levels of MCI subjects are positively correlated with cognitive performances as measured by mini-mental status examination (MMSE) and negatively correlated with CSF tau levels, suggesting the involvement of miR-195-5p in early development of AD with a potential impact on cognition. Similar differences in miR-195-5p levels are seen in ApoE4^{+/+} mouse hippocampal brain tissue and cultured neurons when compared to ApoE3^{+/+} counterparts. Over-expressing miR-195-5p reduces expression levels of its top predicted target synaptojanin 1 (synj1), a brain PIP₂-degrading enzyme. Furthermore, elevating miR-195-5p ameliorates cognitive deficits, amyloid plaque burden, and tau hyper-phosphorylation in ApoE4^{-/+} mice. In addition, elevating miR-195-5p rescues AD-related lysosomal defects in inducible pluripotent stem cells (iPSCs)-derived brain cells of ApoE4^{+/+} AD subjects while inhibiting miR-195-5p exacerbates these phenotypes. Together, the data described herein provides a regulatory mechanism of miR-195-5p targeted at ApoE4-associated brain PIP₂ dyshomeostasis, cognitive deficits, and AD pathology.

Introduction. The apolipoprotein E4 (ApoE4) allele has been identified as a major risk factor for Alzheimer's Disease (AD) (Mayeux R., Annu Rev Neurosci 2003). Numerous studies suggest that ApoE4 effects Aβ clearance (Fagan A M et al., Neurobiology of disease 2002, Jiang Y et al., Acta Neurochir Suppl 2008, Jordan J et al. The Journal of neuroscience 1998, Ma J et al., Nature 1994, Sharman M J et al., Journal of Alzheimer's disease 2010, Yang D S et al., Journal of neurochemistry 1997, and Fagan A M et al., Neurobiology of disease 2002), neurofibrillary tangle burden (Oyama F et al., Brain research Molecular brain research 1995, Shi Y et al., Nature 2017, Tiraboschi P et al., Neurology 2004, and Wang Y et al., Nat Rev Neurosci 2016), synaptogenesis and synaptic plasticity (Love S et al., Neurobiology of aging 2006, Nathan B P et al., Science 1994, Teter B et al., Journal of neurochemistry 1999, and Trommer B L et al., Neuroreport 2004), glial activation and neuro-inflammation (Keren-Shaul H et al., Cell 2017, Yeh F L et al., Neuron 2016, and Zhu Y et al., Glia 2012). Moreover, ApoE proteins play important roles in lipid metabolism and neuronal homeostasis (Huang Y et al., Neurobiology of disease 2014). Prior studies reveal distinct alterations in brain membrane phospholipid composition, metabolism, and selected enzyme activities in postmortem AD brains (Pettegrew J W et al., Neurochemical research 2001, Mandal P K et al., Neurochemical research 2004, Kanfer J N et al., Neurochemical research 1993, Kanfer J N et al., J Lipid Mediat Cell Signal 1996, and Chan R B et al., J Biol Chem 2012), that can be exacerbated by ApoE4 (Klunk W E et al., Neurobiology of aging 1998). It has been reported that ApoE proteins are important determinants of brain phosphoinositol biphosphate (PIP₂) homeostasis, and the ApoE4 isoform is dysfunctional in this process contributing to the increased susceptibility of cognitive decline in AD (Zhu L et al., Proc Natl Acad Sci USA 2015). It has also been shown that brain PIP₂ levels are lower in ApoE4 brains and neurons due to the increased expression of a PIP₂-degrading enzyme, synaptojanin 1 (synj1) (Zhu L et al., Proc Natl Acad Sci USA 2015).

The functional roles of PIP₂ and synj1 is implicated in AD pathogenesis (Zhu L et al., Proc Natl Acad Sci USA 2015, Berman D E et al., Nat Neurosci 2008, Voronov S V et al., Proc Natl Acad Sci USA 2008, McIntire L B et al., The Journal of neuroscience 2012,

Zhu L et al., The Journal of biological chemistry 2013, Cao J et al., Sci Rep 2017, and Miranda A M et al., Cell Rep 2018). For example, increased expression of synj1 is linked to early endosome enlargement (Cossec J C et al., Human molecular genetics 2012), and ApoE4-associated cognitive deficits in AD (Zhu L et al., Proc Natl Acad Sci USA 2015). The reduction of synj1 provides several beneficial effects in AD such as accelerating Aβ clearance via the lysosomal degradation pathway (Zhu L et al., The Journal of biological chemistry 2013), ameliorating mild traumatic brain injury (TBI)-induced elevation in tau hyper-phosphorylation (Cao J et al., Sci Rep 2017), and rescuing ApoE4-associated cognitive impairments (Zhu L et al., Proc Natl Acad Sci USA 2015). However, molecular signaling mechanisms that link ApoE4 with brain PIP₂/synj1 pathways and impact on cognitive function remain elusive. Increased synj1 levels in ApoE4 brains are partially due to a failure in efficient synj1 mRNA degradation (Zhu L et al., Proc Natl Acad Sci USA 2015). mRNA stability is often regulated by micro-RNA (miRNA) binding to 3′-UTR regions of mRNA (Fabian M R et al., Annu Rev Biochem 2010). It was assessed whether brain synj1 expression may be differentially regulated by ApoE isoforms through miRNA modulation.

Several miRNAs have been previously implicated in various AD processes (Wang M et al., Frontiers in genetics 2019). Moreover, changes in various brain miRNA levels between AD subjects and normal-aged controls have been reported (Goodall E F et al., Frontiers in cellular neuroscience 2013, and Lukiw W J et al., Neuroreport 2007). The existing miRNA datasets from the Religious Orders Study and the Rush Memory and Aging Project (ROSMAP) (Bennett D A et al., Neuroepidemiology 2005, and A Bennett D et al., Current Alzheimer Research 2012) were used in combination with miRNA array studies that were performed in ApoE4⁺ and ApoE4⁻ cortical neurons. A miRNA, miR-195, was identified being differentially expressed between ApoE4⁺ and ApoE4⁻ carriers and targeted at synj1 mRNA as predicted by multiple bioinformatics databases including mirDB (Wong N et al., Nucleic acids research 2015, and Wang X, Bioinformatics 2016). The changes in miR-195 levels are further validated using postmortem human and mouse brain tissue as well as cultured neurons. Furthermore, a regulatory role of miR-195 was characterized in ApoE4-associated brain PIP₂ dyshomeostasis, cognitive deficits and AD pathology.

Materials and Methods. Human miRNA expression profile and data preprocessing. The miRNA expression profile was downloaded from the ROSMAP study (Synapse doi:10.7303/syn3388564). miRNAs that had a call rate less than 95% and an absolute value of lower than 15 in less than 50% of the samples were removed. miRNA expression values were normalized using a variance stabilization normalization method. Cartridges were specified as batches and were corrected with the Combat function in the R package sva (V3.20.0). The data pre-processing resulted in 309 miRNAs in 511 samples. Pre-processed RNA-seq FPKM gene expression abundance data were also downloaded from the ROSMAP study (Synapse doi:10.7303/syn3388564). Genes with at least 1 FPKM in at least 10% of the samples were selected, and data were then corrected for confounding factors including batch, PMI and RIN scores. The pre-processed gene expression profile contains 16,235 genes and 619 samples.

Differential expression and miRNA-gene correlation analysis. Differential expression analysis was performed on the miRNAs between 111 APOE4^{+/+} (ε3/3) and 24 APOE4^{+/-} (ε3/4) carriers using the R package limma (V3.34.0) (Smyth G K, Stat Appl Genet Mol Biol 2004). Multiple tests were adjusted using the Benjamini-Hochberg's (BH) FDR method. Correlation analysis was performed between miRNAs and genes using spearman's correlation test. The miRNA-gene correlation was also examined in each of the subgroups of AD diagnosis, sex and APOE genotype.

miRNA array studies of mouse primary neuron samples. Embryonic 17 days old ApoE^{-/-} cortical neurons were cultured for 7 days in vitro in the presence of conditioned media derived from ApoE3 and ApoE4 primary astrocyte cultures (Zhu L et al., Proc Natl Acad Sci USA 2015, and Zhu L et al., J Biol Chem 2013) (N=3/group). miRNAs were extracted using miRCURY extraction kits (Exiqon Inc.) and then labeled using miRCURY LNA microRNA Hi-Power Labeling kit, Hy3/Hy5 and hybridized on the miRCURY LNA microRNA Array. The quality assessment using control spike-in oligo nucleotides produced signals in expected range indicated successful labeling. Following normalization of quantified signals after background correction using global Lowess regression algorithm, unsupervised and supervised data analysis were performed. The microRNA profiling identified a subset of microRNAs that are differentially expressed in the ApoE3 versus ApoE4-treated neurons.

miRNA target prediction. Targets of the miRNAs were predicted with the R package multiMiR (V2.2.0), which is a miRNA-target interaction database complying nearly 50 million human and mouse data from 14 different databases (Ru Y et al., Nucleic acids research 2014).

Human brain and CSF sample preparation. Equal amounts of postmortem human parietal cortex brain tissues (50 μg by net weight) from the NIH brain and tissue repository (NBTR), as well as cerebrospinal fluid (1 ml by net weight) from mild cognitive impairment (MCI) subjects enrolled in the James J Peters VAMC study (“Markers of Transition to AD in the Veterans with MCI”) and Icahn School of Medicine at Mount Sinai (ISMMS) AD Research Center participants were used for studies.

Animal models. Human ApoE4^{+/+} or ApoE3^{+/+} knock-in (KI) mouse models without (Grootendorst J et al., Behavioural brain research 2005, Rodriguez G A et al., Learn Mem 2013, and Wang C et al., Neurobiology of disease 2005) or with 5×FAD background (Balu D et al., Neurosci Lett 2019, and Tai L M et al., J Lipid Res 2017) were genotyped (Zhong L et al., Mol Neurodegener 2016). Sex as a biological variable was taken into considerations with inclusion of both male and female mice in the experiments.

Phospholipid analysis. Samples were subjected for lipid extraction, followed by the quantification by anion-exchange HPLC (Zhu L et al., The Journal of biological chemistry 2013, and Nasuhoglu C et al., Anal Biochem 2002).

Mouse Neuronal and human iPSC culture. Primary cortical neurons were cultured (Zhu L et al., Proc Natl Acad Sci USA 2015, and Zhu L et al., J Biol Chem 2013) before being fixed and stained for confocal microscopy analysis (Zeiss LSM) (Berg I et al., Nat Protoc 2010). In some conditions, bovine serum albumin (BSA) or receptor-associated protein (RAP) was included in the cultured media (N=3/group). Alternatively, cultured neurons were transfected with adeno-associated virus 2 (AAV2)-containing miR-195-5p or miR-374 or scramble controls for 5 days before subjected to analysis. The ApoE4^{+/+} and ApoE3^{+/+} iPSCs were differentiated into neural progenitor cells (NPCs) by dual SMAD inhibition followed by neural rosette selection and forebrain-specific patterning by 20 ng/ml FGF2 exposure (Bowles K R et al., PloS one 2019, and Tcw J et al., Stem cell reports 2017). These NPCs were purified by MACS for CD271⁻/CD133⁺ (Bowles K R et al., PloS one 2019) and differentiated to cortical neurons (Bardy C et al., Proc Natl Acad Sci USA 2015, and Paquet D et al., Nature 2016) and a homogeneous population of astrocytes (Tcw J et al., Stem cell reports 2017, and Shaltouki A et al., Stem Cells 2013) before subjected to viral transfection and confocal microscopy analysis. Alternatively, mouse cortical neurons derived from ApoE4^{+/+} and ApoE3^{+/+} KI mice with synj1^{+/+} or synj1^{-/-} genotypes were co-cultured with ApoE4^{+/+} and ApoE3^{+/+} iPSC-derived pure astrocytes. In some experiments, cultured neurons were transfected with AAV2-containing miR-195-5p, scramble controls or miR195 inhibitors that specifically prevent miR-195-5p binding to its target mRNA before co-culturing with iPSC-derived astrocytes. Cultured iPSC-derived neurons and astrocytes were then incubated with lysotracker red for various time periods before fixation and double-stained for pTau and a nuclear marker DAPI (blue) for confocal microscopy analysis (Zeiss). N=4-5/condition.

Stereotaxic injection and behavior studies. Eight to nine weeks old male and female ApoE3^{+/+} and ApoE4^{+/+} KI mice without 5×FAD background (N=19-23/group), or with 5×FAD background (N=15-17/group) were placed in the stereotaxic apparatus with AAV2 or scramble virus administered into the dorsal CA1 regions of bilateral hippocampal brain regions using pressure injection (Labonte B et al., Nat Med 2017, and Passini M A et al., The Journal of neuroscience 2006). Injection volumes (0.5-2.0 μl) were delivered over 10 min to avoid tissue damage. Six to nine months after viral delivery, mice were tested with the NOR task (Zhu L et al., Proc Natl Acad Sci USA 2015, Elder G A et al., J Neurotrauma 2012, and Howlett D R et al., Brain Res 2004). Mice were randomized for genotype and sex, and blinded throughout the behavior data collection and analysis, surgical manipulations, and sample collection. Animals were excluded from behavior analysis if the total exploration time was less than 4 seconds or if they had an illness that prevented them from reliably completing the behavior tests.

Brain and neuronal sample preparation and biochemical analysis. Snap-frozen mouse hemi-brains or cultured neurons were harvested in lysis buffer (Lane R F et al., The Journal of neuroscience 2010) and processed via step-wise solubilization (Lane R F et al., The Journal of neuroscience 2010, and Kawarabayashi T et al. The Journal of neuroscience 2001) followed by SDS-PAGE to determine levels of synj1, dyn1, holoAPP, and CTFs. Levels of Aβ₄₂, Aβ₄₀, pTau, Tau and ApoE were determined using high-sensitive ELISA kits. Some tissue was used for miRNA and RNA extraction followed by qPCR and RNA-seq analysis. Some animals underwent perfusion followed by brain tissue section for immunohistochemical staining of amyloid plaque, synj1 and pTau.

Differential gene expression analysis for miR-195-5p treated mice. The RNA-seq samples collected from mouse brains were profiled on the Illumina HiSeq platform. Quality control of generated reads was performed using FASTQC (0.11.8). The raw sequencing reads were aligned to the GRCm38 mouse genome (release 95) using star aligner (V2.5.0b). Following the read alignment, gene expression was quantified at the gene level based on Ensembl gene model GRCm38.95 using FeatureCounts (Liao Y et al., Bioinformatics 2014). Genes with at least one count per million (CPM) in the samples were considered as expressed and hence retained for further analysis. The trimmed mean of M-values normalization method (Robinson M D et al., Genome Biol 2010), was used to adjust for sequencing library size differences. Differential expression analysis was then performed on the quality controlled and normalized gene expression data using the R package limma (V3.34.0). The comparisons were carried out between miR195-treated and scramble control samples stratified by sex and APOE genotype. Multiple tests were adjusted using the BH FDR method.

Functional enrichment analysis. The functional enrichment analysis was carried out for genes significantly correlated with miRNAs in human ROSMAP dataset, predicted target genes of each miRNA of interest, and differentially expressed genes identified from miR195-5p-treated mouse RNA-seq dataset. These genes were queried against the molecular signatures database (MSigDB v6.1) using Fisher's Exact Test and gene set enrichment analysis (GSEA) (Subramanian A et al., Proc Natl Acad Sci USA 2005).

Antibodies and reagents. The anti-synj1 (rabbit polyclonal Ab, Novus, RRID: AB_11047653), anti-pTau AT8 and Tau-5 (ThermoFisher, RRID: AB_223647 and 10980631), anti-Rab5 (Santa Cruz Biotechnology, RRID:AB_628191), anti-β actin and tubulin (Santa Cruz Biotechnology, RRID:AB_476697 and 477498), anti-holoAPP MAB348 and 6E10 (Millipore RRID:AB_94882 and 564201), anti-beta-Amyloid (Cell Signaling Technology, RRID: AB_2056585), anti-MAP2 (Abcam, RRID:AB 297885), anti-dynamin clone 41 for (BD bioscience; RRID:AB_3976413), anti-mouse and rabbit HRP (ThermoFisher, RRID:AB_2556542 and 2540618), Texas-Red and Alexa₅₅₅ conjugated anti-mouse and rabbit IgG (ThermoFisher, RRID:AB_10374713, 10983944, 2535987 and 1090271) were purchased. AAV2-containing miR-195-5p, miR-374, scramble controls and miR-195 inhibitors were generated and obtained from ABM Inc. with detailed sequence information available (Am00100, Amm1017200 and Amm3026700). The miRNA extraction kit and qPCR probes for specific miRNAs were purchased from Exiqon Inc. The qPCR probes for actin (Hs1060665_g1), synj1 (Hs00953234_m1 and Mm01210539_m1), gapdh (Mm99999915_g1), RNU6B (NR 002752), 18s and 45s (4331182, Mm03928990_g1) were also purchased from ThermoFisher.

Statistical analysis. The sample sizes of each experiment were chosen based on power calculations derived from previous similar studies which allowed the determination of group sizes needed to achieve statistically significant results. The experiments including controls were performed in randomly assigned groups. Experimenters were blinded to the experimental condition of the animal while conducting experiments. The conditions were revealed after quantification was completed. Levels of miR-195-5p and miR-374 were normalized to U6 and RNU6B (internal controls) while synj1 mRNA normalized to GAPDH and 18s, and then expressed as Log₂fold of changes when compared to controls. Levels of synj1, dyn1, pTau, Tau, and holoAPP were normalized to β-actin levels and expressed as a percentage of the control. Absolute Aβ₄₂, Aβ₄₀, pTau, Tau, and ApoE concentrations were quantitatively determined by ELISA and expressed as a percentage of the control. Independent-samples t-tests were used to determine significant mean differences (the threshold for significance sets at p<0.05). ANOVA with post-hoc tests were used to determine group differences for multiple comparisons. Pearson correlation coefficients were calculated to determine the linear relationship between the two variables. Equality of variance was checked for statistical comparisons. When independent-samples (-tests were used and equality of variances of compared groups were not the same, the Welch's corrections were applied. Statistical analysis was performed using Prism 8.0.

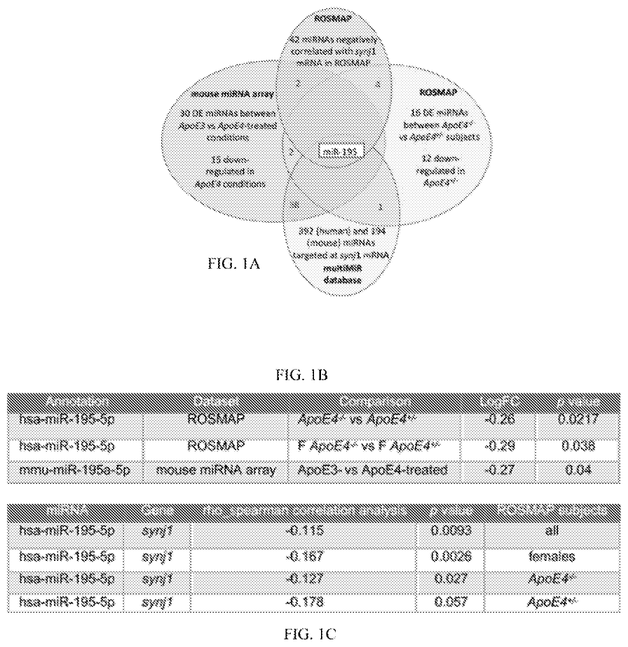

Results. miR-195 is identified as a top candidate miRNA involved in APOE-regulated synj1 expression. First, differential expression analysis was performed between ApoE4^{-/-} (ε3/ε3; N=111) and ApoE4^{+/-} (ε3/ε4; N=24) carriers on human miRNA profiles in ROSMAP datasets. Sixteen significantly differentially expressed miRNAs (p<0.05) were identified, twelve of which with reduced expression in the ApoE4^{+/-} carriers (FIG. 1A). In parallel, miRNA array studies of ApoE^{-/-} hippocampal neurons treated with ApoE3 or ApoE4-conditioned media (CM) was performed (FIG. TA). Thirty significantly differentially expressed miRNAs (p<0.05) were identified, fifteen of which with reduced expression in the ApoE4-treated conditions (FIG. 6A). Among these miRNAs, miR-195 is differentially expressed miRNA between ApoE4⁺ and ApoE4⁻ conditions that is commonly shared between human and mouse datasets (hsa-miR-195-5p and mmu-miR-195a-5p; FIG. 1B). Another miRNA, miR-155 is also identified in both human and mouse datasets (hsa-miR-155 and mmu-miR-155-5p) but in opposite trends (higher in human ApoE4^{+/-} carriers and lower in mouse ApoE4-treated conditions).

Next, prediction of miRNAs that putatively bind with synj1 mRNA using 14 compiled multiMiR database (Ru Y, et al. Nucleic acids research 2014; 42(17): e133-e133) was performed. From a total of 392 (human)/194 (mouse) miRNAs targeted at synj1 mRNA (FIG. 1A), miR-195-5p was predicted as a top candidate in the human mirDB database (Wong N et al., Nucleic acids research 2015, and Wang X, Bioinformatics 2016) (predicting score: 99.9/100). MiR-195 was also predicted as a top candidate miRNA targeted at synj1 in several other databases (FIG. 6B) such as elmmo (predicting score: 0.80/1), and diana_micro (predicting score: 0.79/1).

Furthermore, the correlation between synj1 mRNA and the miRNAs in ROSMAP dataset were examined using Spearman's correlation test. MiR-195-5p is negatively correlated with synj1 mRNA levels in human subjects (FIG. 1C: r=−0.115, p=0.0093). Similarly, negative correlations between miR-195-5p and synj1 mRNA levels are seen in female subjects (r=−0.167, p=0.0026) and in ApoE3^{+/+} carriers (r=−0.127, p=0.027). A negative correlation trend can be seen in ApoE4^{+/-} carriers as well but with no statistical significance (r=−0.178, p=0.057), suggesting a possible weakened or perturbed network regulation between miR-195 and synj1 in the presence of ApoE4 allele. Separately, miR-374b also showed negative correlation with synj1 mRNA (r=−0.09, p=0.04). Additionally, mmu-miR-374b-5p is differentially expressed between ApoE3 and ApoE4-treated conditions but the differences in hsa-miR-374-5p levels between human ApoE4^{+/-} and ApoE4^{-/-} carriers are not statistically significant (p=0.188).

To better understand the molecular pathways miR-195-5p regulates, the functions of predicted miR-195-5p targeted genes and those significantly correlated with miR-195-5p in the ROSMAP dataset were investigated. The top enriched functions for target genes and genes negatively correlated with miR-195-5p include regulation of neuronal and synaptic function, neurogenesis, and differentiation, while functions of genes positively correlated with miR-195-5p are enriched in the circulatory system and vasculature development.

Together, these results show a role of miR-195-5p as a top candidate miRNA in regulating the ApoE-synj1-PIP₂ pathways.

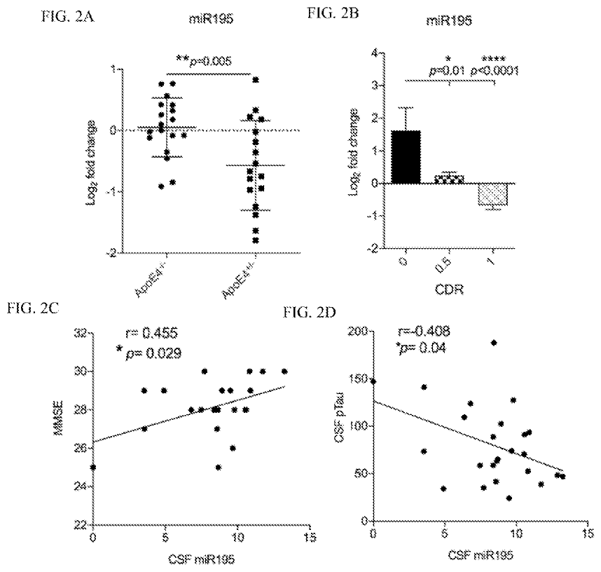

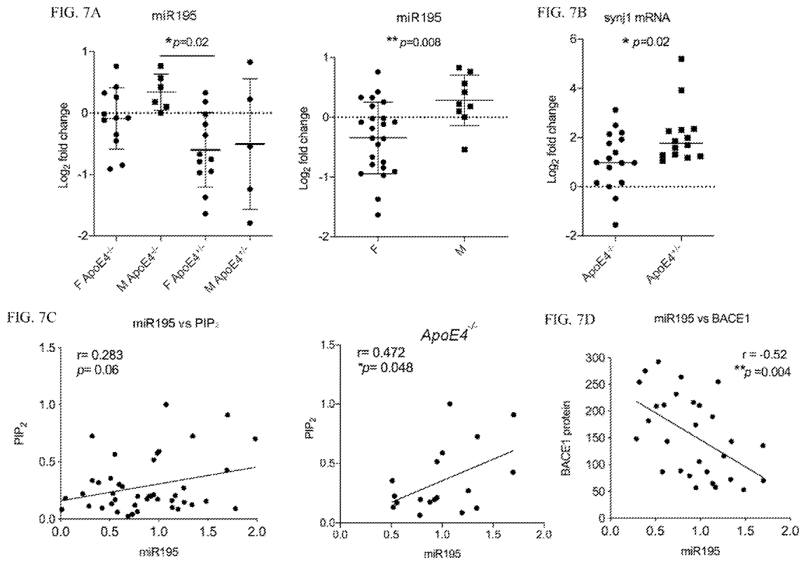

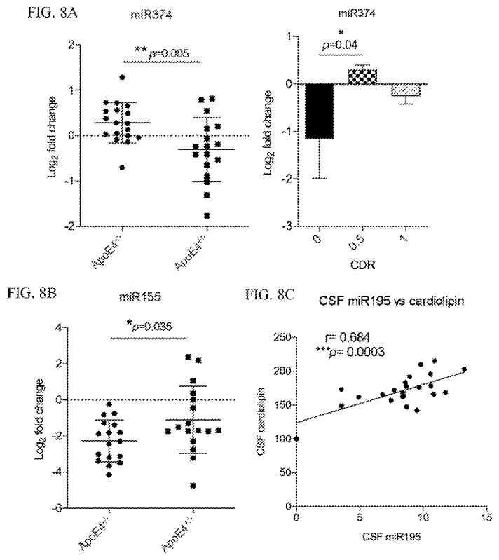

Reduction of brain miR-195-5p levels is associated with ApoE4 genotype, disease progression and cognitive decline. To validate differential expression patterns of miR-195 between ApoE4^{+/-} and ApoE4^{-/-} carriers, miR-195-5p levels were examined in human brain tissue and CSF samples. It was found that miR-195-5p levels were reduced in parietal cortex tissues derived from ApoE4^{+/-} mild cognitive impairment (MCI) and early AD subjects with clinical dementia rating (CDR) scores between 0.5 and 1 compared to levels in ApoE4^{-/-} donors (FIG. 2A). Interestingly, a pattern of reduction in miR-195-5p levels was observed along with disease progression from normal aging to MCI and early AD (FIG. 2B), similar to what was previously seen with PIP₂ and phosphoinositol (PI) changes in early AD development (Zhu L et al., Proc Natl Acad Sci USA 2015). A significant reduction in miR-195-5p was found in female subjects compared to male subjects (FIG. 7A), with differences also noted between male ApoE4^{-/-} versus female ApoE4^{+/-} subjects. Consistently, a reciprocal elevation of synj1 mRNA levels was observed in ApoE4^{+/-} subjects when compared to levels in ApoE4^{-/-} subjects (FIG. 7B). A positive correlation was noted between brain miR-195-5p and PIP₂ levels in ApoE4^{-/-} carriers with CDR 0.5-1, and a positive correlation trend was seen in CDR 0-1 subjects regardless of ApoE genotypes (FIG. 7C). No correlation was seen between brain miR-195-5p and other phospholipid species, e.g., PI and phosphoinositol phosphate (PIP). No correlation was seen between brain miR-195-5p and other variables such as post-mortem interval (PMI) and age. A negative correlation was observed between brain miR195 and another known target of miR-195, beta-secretase 1 (BACE-1) (Zhu H C et al., Brain Res Bull 2012) expression in the CDR 0.5-1 cohort (FIG. 8D). However, no correlation between miR-195 and Aβ levels was observed.

There were statistically significant differences in human miR-374 levels between ApoE4^{+/-} and ApoE4^{-/-} subjects (hsa-miR374-5p; FIG. 8A). However, there were no significant changes in miR-374 levels along the disease progression except a transient elevation at MCI stage (FIG. 8A). Consistently with ROSMAP data, statistically significant differences in human miR-155 levels with higher levels in ApoE4^{+/-} subjects (hsa-miR155-5p; FIG. 8B) was observed. No significant differences were seen in miR-155 levels along disease progression. Moreover, no significant differences were seen in miR-195-5p or miR-374 levels between ApoE4^{+/-} and ApoE4^{-/-} subjects of normal aging or advanced AD either (CDR 0 or 3 and above), suggesting the functional involvement of miR-195-5p in early disease development and acceleration by ApoE4 genotype.

Using cerebrospinal fluid (CSF) samples from a cohort of MCI subjects with CDR 0.5 (MCI defined by clinical examination and neuropsychological assessments), it was found that CSF miR-195-5p levels were positively correlated with cognitive performance measured by mini-mental status examination (MMSE; FIG. 2C), and negatively correlated with total tau levels (FIG. 2D). While CSF PIP₂ levels were below the detectable range, a positive correlation was seen between CSF cardiolipin and miR-195-5p (FIG. 8C), suggesting a potential involvement of miR-195-5p in mitochondrial function (Monteiro-Cardoso V F et al., Journal of Alzheimer's disease 2015, and Chicco A J, et al., American journal of physiology Cell physiology 2007).

Together, these results show that reduction of brain and CSF miR-195-5p levels is associated with ApoE4 genotype, cognitive decline, and tau pathology during early AD development.

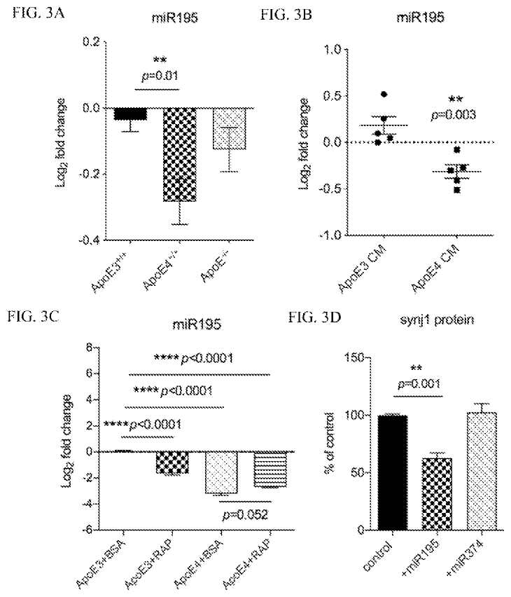

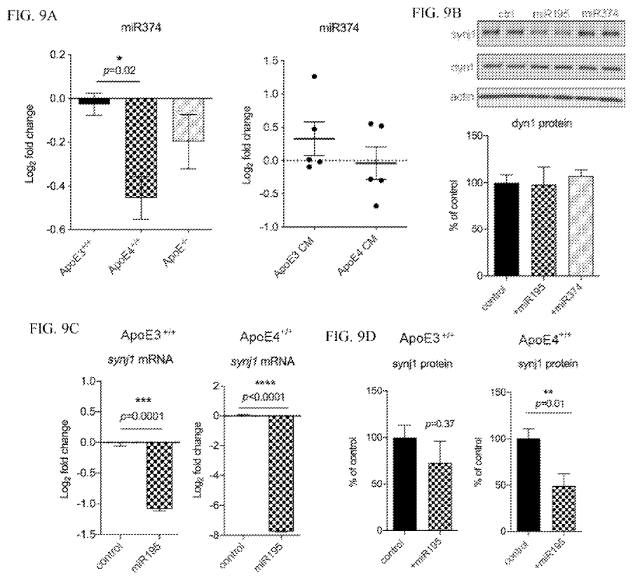

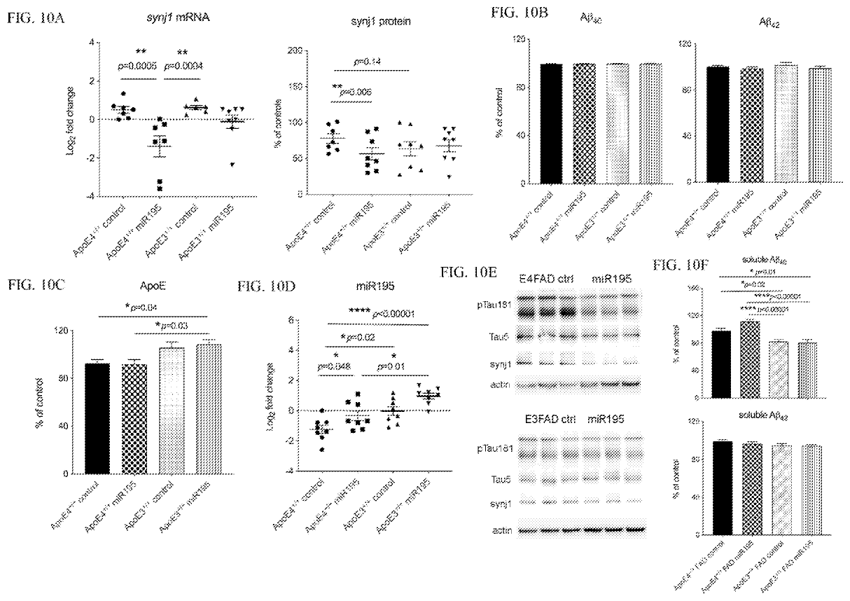

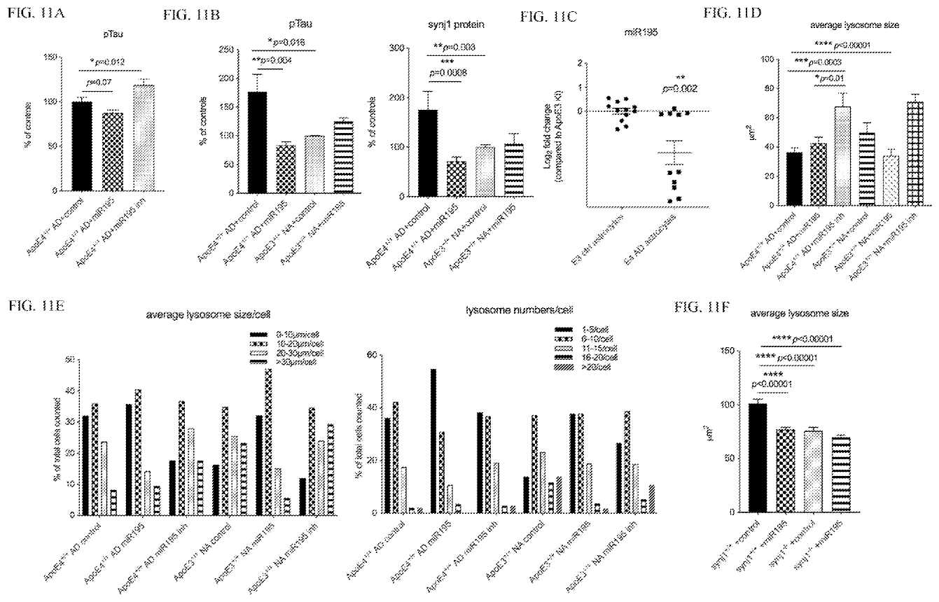

MiR-195-5p expression is reduced in ApoE4 mouse brains and cultured neurons. Next, it was investigated whether differences in miR-195-5p levels can be recapitulated in mouse models and primary neurons. It was found that the levels of miR-195-5p were lower in 12-month old ApoE4^{+/+} mouse brains compared to ApoE3^{+/+} mice (FIG. 3A). A nominal reduction in miR-195-5p levels was seen in ApoE^{-/-} brains. Similarly, miR-374 was decreased in ApoE4^{+/+} mouse brains when compared to ApoE3 conditions (FIG. 9A) with a nominal reduction in ApoE^{-/-} mice. In cultured ApoE^{-/-} hippocampal neurons, levels of miR-195-5p were consistently lower with ApoE4 CM from astrocytes compared to those with ApoE3 CM (FIG. 3B). A nominal difference was noted in miR-374 levels in neurons treated with ApoE4 CM when compared to those treated with ApoE3 CM (FIG. 9A) but failed to achieve statistical significance due to large variations among samples.

Treatment of ApoE-receptor associated protein (RAP), an inhibitor of ApoE receptors (LaDu M J et al., Neurochemistry international 2001, and Qiu Z et al., J Biol Chem 2004) abolished differential expression patterns of miR-195-5p relative to control (BSA: bovine serum albumin). The RAP treatment in the presence of ApoE3-CM led to a reduction in miR-195-5p levels (FIG. 3C), whereas in ApoE4-CM treated conditions, miR-195-5p levels were much lower at baseline with a trend of improvement following RAP treatment. These results suggest that astrocyte-derived ApoE likely binds to ApoE receptors on neurons leading to changes in neuronal miR-195-5p and ApoE4 exhibits loss-of-function effects on neuronal miR-195-5p expression.

Next, it was determined whether up-regulation of miR-195-5p in ApoE4 conditions could modulate expression levels of its predicted target gene, synj1. Over-expression of miR-195-5p but not miR-374 significantly reduced synj1 protein levels in ApoE-neurons treated with ApoE4 CM (FIG. 3D; synj1 levels with miR-195-5p: 62.87±4.48% of controls, p=0.001; with miR-374: 102.4±7.77% of controls, p=0.93). No changes were seen in expression levels of another endocytic adapter protein dynamin 1 (dyn1; FIG. 9B), suggesting a specific effect of miR-195-5p on synj1 expression. Similarly, miR-195 over-expression in ApoE3^{+/+} or ApoE4^{+/+} neurons resulted in synj1 expression reduction in both mRNA and protein levels (FIGS. 9C and D). It should be noted that ApoE4^{+/+} neurons exhibited more dramatic changes with over-expression of miR-195-5p in synj1 mRNA (ApoE3^{+/+} w miR-195-5p log₂FC: −1.084±0.035 versus ApoE4^{+/+} w miR-195-5p: −7.751±0.043; FIG. 9C), and protein levels (ApoE3^{+/+} w miR-195-5p 70.9±21.2% versus ApoE4^{+/+} w miR-195-5p 48.0±9.84% of controls; FIG. 9D) than ApoE3^{+/+} neurons, possibly due to much lower baseline levels in ApoE4^{+/+} cells making them more sensitive to miR-195-5p manipulations.

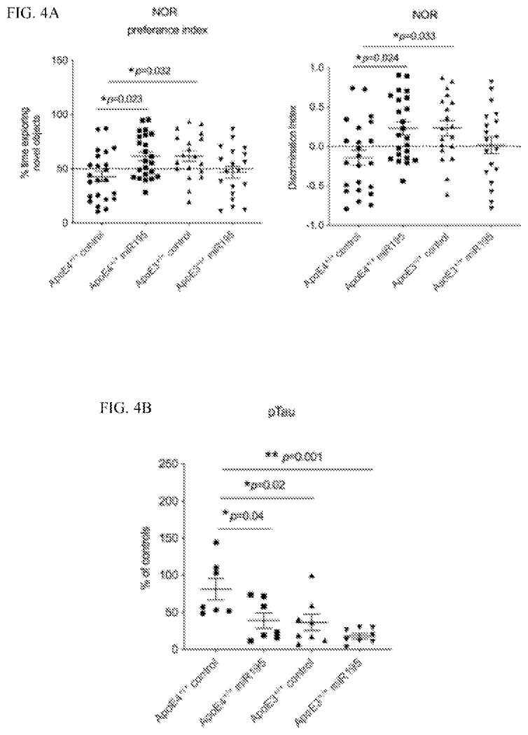

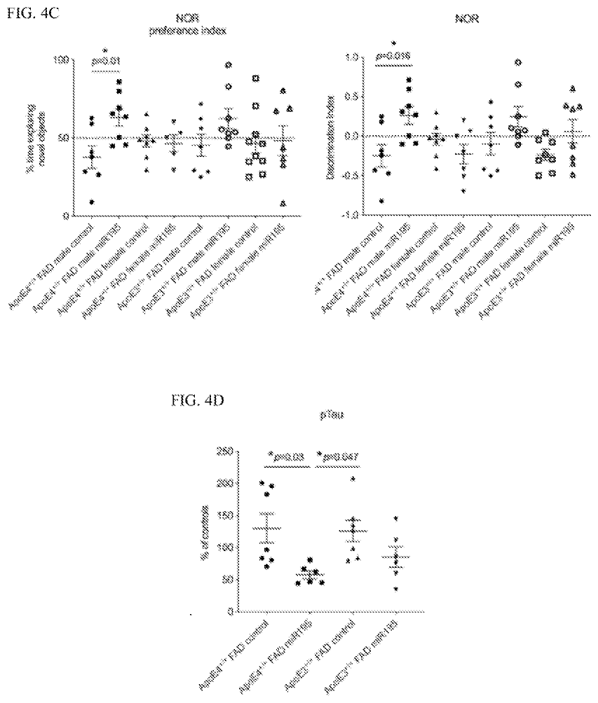

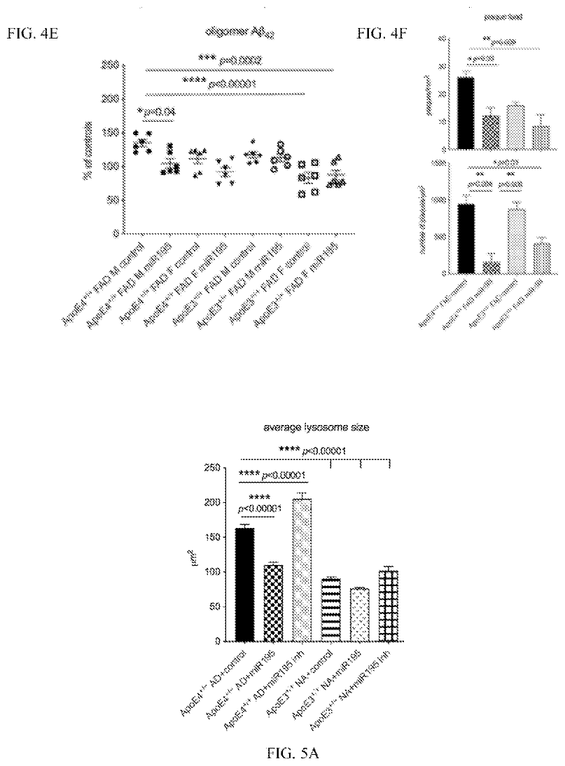

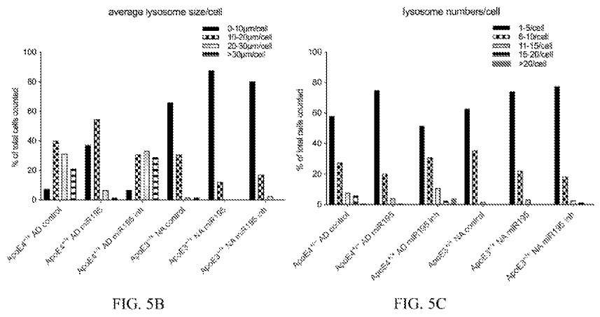

Over-expression of miR-195-5p rescues cognitive deficits and ameliorates AD-associated pathologies in ApoE4 mouse models. Next, it was determined if over-expressing miR-195-5p could rescue ApoE4-associated cognitive dysfunction in vivo using ApoE4^{-/+} and ApoE3^{+/+} KI mice without and with AD transgenic background. It was previously demonstrated that male human ApoE4^{+/+} KI mice manifested memory impairments as measured by novel object recognition (NOR) tests with an inability to discriminate between novel and familiar objects²⁶. Here it was found that ApoE4^{+/+} KI mice spent less time exploring novel objects than ApoE3^{+/+} KI mice did (FIG. 4A preference index: ApoE4^{+/+} versus ApoE3^{+/+} scramble controls: 42.9% versus 61.7%, p=0.032), consistent with previously observations²⁶. This deficit was completely abolished by viral delivery of miR-195-5p bilateral hippocampi of ApoE4^{+/+} KI mice (FIG. 4A, 61.5%, p=0.023 when compared to ApoE4^{+/+} scramble controls). However, no statistically significant differences were seen between scramble and miR-195-5p over-expressing ApoE3^{+/+} animals. Moreover, the discrimination index studies using the difference in exploration time for novel versus familiar object (Antunes M et al., Cognitive processing 2012) showed consistent results suggesting that impaired discrimination behaviors in ApoE4^{+/+} KI mice were completely rescued by miR-195-5p over-expression (FIG. 4A discrimination index: ApoE4^{+/+} versus ApoE3^{+/+} scramble controls: −0.144 versus 0.234; p=0.033; ApoE4^{+/-} scramble controls versus ApoE4^{+/+} miR-195-5p: −0.144 versus 0.231; p=0.024). The total amount of exploration time was comparable among groups.

Over-expressing miR-195 also reduced brain phospho-Tau (pTau) levels in ApoE4 mouse brains (FIG. 4B; ApoE4^{+/+} scramble controls versus ApoE4^{+/+} miR-195-5p: 81.3 versus 39.1% of controls; p=0.04). Consistently, levels of synj1 mRNA and protein levels were reduced in ApoE4^{+/+} mouse brains with miR-195-5p over-expression (FIG. 10A). Trends of reduction but to lesser degrees in pTau, synj1 mRNA and protein levels were seen in ApoE3^{+/+} mice with miR-195-5p over-expression. No significant changes were seen in endogenous Aβ₄₀, Aβ₄₂ or ApoE levels with over-expression of miR-195-5p in ApoE4^{+/+} or ApoE3^{+/+} mouse brains (FIGS. 10B and 10C). qPCR confirmed elevated miR-195-5p levels after viral manipulations (FIG. 10D).

Similar experiments were performed in ApoE4^{+/+} or ApoE3^{+/+} mouse models with 5×FAD background with AD-related pathological, neuro-inflammatory, and behavioral phenotypes manifesting at 4-8 months of age (Balu D et al., Neurosci Lett 2019, and Tai L M et al., J Lipid Res 2017). Sex dimorphic responses were noted in these mouse models with male ApoE4^{+/+} FAD mice being most sensitive to miR-195-5p manipulations (FIG. 4C preference index: ApoE4^{+/+} FAD scramble controls versus miR-195-5p: 37.8% versus 63.1%; p=0.01; discrimination index: ApoE4^{+/+} scramble controls versus miR-195-0.245 versus 0.262; p=0.016). p-Tau reduction was also observed in ApoE4^{+/+} FAD mice with miR-195-5p over-expression (FIGS. 4D and TOE). Similar changes were seen in total Tau levels in ApoE4^{+/+} FAD miR-195-5p over-expression mouse brains. A dramatic reduction in brain oligomer Aβ₄₂ measured by ELISA (FIG. 4E) and amyloid plaque burden determined by plaque numbers and plaque density (FIG. 4F) in ApoE4^{+/+} and ApoE3^{+/+} FAD mouse brains was found with miR-195-5p over-expression. However, no significant changes were seen in levels of soluble Aβ₄₀, Aβ₄₂(FIG. 10F), holo-APP or BACE-1 after miR-195-5p over-expression.

It was also examined if miR-195-5p over-expression leads to changes in gene expression patterns and downstream pathways in EFAD mouse brains. Again, most differentially expressed genes (DEGs) are enriched in regulation of neuron, synapse, and immune functions. Further GSEA studies Subramanian A, et al. Proc Natl Acad Sci USA 2005; 102(43): 15545-15550) suggested top pathways perturbed by miR-195-5p over-expression are mitochondrial related pathways, consistent with studies in human brain dataset with top pathways enriched for genes negatively correlated with miR-195-5p involved in mitochondrial function.

Together, these results suggest that elevating miR-195-5p levels in ApoE4 mouse models without and with AD background can rescue ApoE4- and AD-related cognitive deficits and pathological changes.

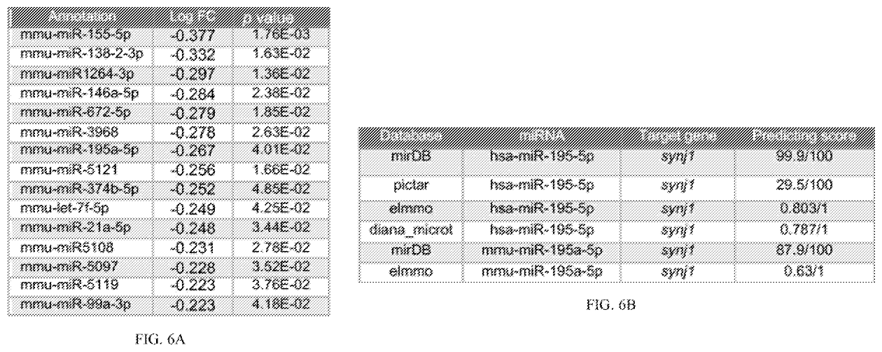

Over-expression of miR-195 endo-lysosomal defects in iPSC-derived brain cells of ApoE4 AD subjects. Next, it was investigated if manipulating miR-195-5p levels could ameliorate AD-related pathologies using human induced pluripotent stem cells (hiPSCs)-derived neuron and astrocyte co-culture from ApoE4^{+/+} AD subjects and ApoE3^{+/+} normal aging subjects (TCW et al., bioRxiv; https://doi.org/10.1101/713362). At baseline, ApoE4^{+/+} neurons (human iPSC or mouse) manifested enlarged lysosomes and increased numbers of lysosomes within each cell when compared to ApoE3^{+/+} counterparts (FIGS. 5A-C). The average size of lysosomes measured by area was 163.5 μm² in ApoE4^{+/+} versus 90.4 μm² in ApoE3^{+/+} neurons (FIG. 5C, p<0.00001). There were 46.7% of ApoE4^{+/+} neurons with the diameter of lysosomes ranged from 10 to 20 μm, whereas 66.1% of ApoE3^{+/+} neurons with the diameter ranged between 0 to 10 μm. There were 18.4% ApoE4^{+/+} neurons with >10 lysosomes/cell, whereas 1.6% ApoE3^{+/+} neurons with >10 lysosomes/cell. Over-expression of miR-195 in ApoE4^{+/+} neurons led to a significant reduction in lysosome size (109.94 m², p<0.00001). The diameter of lysosomes and numbers of lysosomes per cell in ApoE4^{+/+} neurons after miR-195 over-expression (53.3% of neurons with the diameter ranged between 0 to 10 μm; 1.7% with >10 lysosomes/cell) were also shifted towards the lysosomal phenotypes in ApoE3^{+/+} neurons at baseline. In contrast, treatment with a miR-195 inhibitor exacerbated the lysosomal phenotypes of ApoE4^{+/+} neurons (average size: 205.4 μm², p<0.00001) and increased numbers of lysosomes per cell (40% of neurons with the diameter of lysosomes >30 μm; 25% of cells with >10 lysosomes/cell). No significant differences were seen in ApoE3^{+/+} neurons treated with miR-195-5p over-expression or inhibition when compared to the baseline. The pTau levels were reduced with over-expressing miR-195-5p and increased with miR-195-5p inhibition as demonstrated by fluorescent staining (FIG. 11A) and ELISA (FIG. 11B).

MiR-195-5p levels were also lower in cultured iPSC-derived astrocytes from ApoE4^{+/+} AD subjects when compared to those in ApoE3^{+/+} normal aging (NA) iPSC-derived astrocytes (FIG. 11C, p=0.002). The effects of miR-195-5p inhibitor on lysosomes can also be seen in ApoE4^{+/+} astrocytes with a significant increase in average size of lysosomes (FIG. 11D, control versus miR-195-5p inhibitor treatment 36.5 μm² versus 67.7 μm², p<0.00001; FIG. 11E 8.3% of control astrocytes with the diameter of lysosomes >30 μm versus 17.6% of miR-195-5p inhibitor treated). However, no significant differences were seen in lysosomes of ApoE4^{+/+} astrocytes with miR-195-5p treatment when compared to control, or between ApoE4^{+/+} versus ApoE3^{+/+} astrocytes at baseline.

Similar experiments were performed using mouse synj1^{+/+} and synj1^{-/-} neurons co-cultured with ApoE4^{+/+} iPSC-derived astrocytes in the presence or absence of miR-195-5p over-expression. It was found that genetic knockout of synj1 manifested similar effects on lysosomal phenotypes as miR-195-5p over-expression. The average size of lysosomes was 101.5 μm² in synj1^{+/+} versus 77.0 μm² in synj1^{-/-} neurons (FIG. 11F, p<0.00001). With miR-195 over-expression, the size of lysosomes was reduced to 75.8 μm² in synj1^{+/+} neurons (p<0.00001). However, over-expression of miR-195-5p in synj1^{-/-} neurons did not exhibit any additive effects (69.7 μm²), suggesting that miR-195-5p indeed acts through its target gene synj1 to rescue AD-related lysosomal defects.

Together, these results show that elevating miR-195-5p levels in human iPSC-derived ApoE4^{+/+} AD brain cells can rescue lysosomal defects, whereas inhibiting miR-195-5p can exacerbate these phenotypes.

Discussion AD is a complex, multifactorial neurodegenerative process, and accumulating evidence indicates the importance of miRNAs in AD pathogenesis. The studies described herein characterize the functional involvement of a miRNA, miR-195-5p in ApoE4-associated pathology. More importantly, these data reveal a regulatory role of miR-195-5p in the ApoE4 genotype-associated cognitive and lysosomal defects that contribute to AD development.

Dysregulation in brain miRNAs has been described in human subjects and mouse models of AD with proposed involvement of AP-dependent and AP-independent pathways (Goodall E F et al., Frontiers in cellular neuroscience 2013, Lukiw W J et al., Neuroreport 2007, and Sierksma A et al., Mol Neurodegener 2018). The studies described herein demonstrate miR-195-5p reduction during AD development, which correlates with early disease progression but not with advanced stages of AD. ApoE4 genotype accelerates miR-195-5p reduction, which coincides with cognitive decline and increased tau pathology (FIG. 2). Patterns of changes in phosphoinositol (PI) metabolites correlated with disease conversion from normal aging to early AD (Zhu L et al., Proc Natl Acad Sci USA 2015). The results disclosed herein further indicate miR-195-5p changes are consistent with alterations in brain PIP₂ levels (FIG. 7C). These findings highlight utilizing miR-195-5p levels as surrogate biomarkers to monitor brain PIP₂ homeostasis and cognitive performance and detect early AD development and progression.

Functional enrichment studies of human dataset and mouse transcriptomic dataset implicate the roles of miR-195-5p in regulating neuronal and synaptic function, neurogenesis, and differentiation. The data indicate that restoring miR-195-5p levels in vivo rescues ApoE4-associated cognitive deficits (FIGS. 4A and 4C), ameliorates amyloid plaque burden (FIGS. 4E and 4F) and pTau levels (FIGS. 4B and D), and improves lysosomal defects in cultured human iPSC-derived brain cells (FIG. 5). While no reduction in endogenous mouse Aβ in ApoE KI mouse models (FIG. 10B) or soluble Aβ levels in EFAD mice (FIG. 10F) was found, dramatic decreases in oligomer Aβ levels and plaque burden was observed with over-expression of miR-195-5p (FIGS. 4E and 4F). In addition, a negative correlation was found between miR-195 and BACE1 expression in human brain tissue (r=−0.516, p=0.004; FIG. 7D), consistent with a previous report (Zhu H C et al., Brain Res Bull 2012). However, no changes in BACE-1 levels were seen with miR-195-5p over-expression. Together, these data suggest that miR-195-5p most likely regulates Aβ clearance instead of Aβ generation, and restoration of lysosomal function may facilitate these processes, as indicated in prior reports that synj1 reduction accelerates lysosomal clearance of Aβ (Zhu L et al., J Biol Chem 2013).

Moreover, a functional role of miR-195-5p in regulating pTau levels is shown (FIG. 4 and FIGS. 11A and 11B), consistent with recent findings that down-regulation of synj1 prevents mild TBI-induced tau hyper-phosphorylation (Cao J et al., Sci Rep 2017). It was previously reported that the exosomal secretion of tau may play an important role in tau spread, which could be regulated by miRNAs (Asai H et al., Nat Neurosci 2015). miR-195-5p may serve an important role in modulating tau pathology secondary to impaired clearance through the lysosomal pathway and/or accelerated spread through the exosomal secretory pathway.

The results show that differential regulation of miR-195-5p expression by ApoE isoforms is mediated through binding to ApoE receptors on neurons, and the ApoE4 genotype loses the ability to regulate miR-195-5p levels (FIG. 3C). In addition, these data showing ApoE^{-/-} with lower miR-195-5p expression like ApoE4 (FIG. 3A) further supports loss-of-function effects of ApoE4 on miR-195-5p expression leading to increased synj1 mRNA and protein expression. Over-expression of miR-195-5p in synj1^{-/-} neurons failing to exhibit any additive effects on lysosomal enlargement (FIG. 11F) further strengthens the concepts that miR-195 rescues AD-related phenotypes through its target gene, synj1. It should be noted that ApoE4^{-/-} carriers exhibit higher sensitivity to miR-195-5p manipulations than ApoE4^{-/-} subjects (FIGS. 4 and 10; FIGS. 5 and 11), possibly due to much lower baseline miR-195-5p levels. Furthermore, differences in miR-195-5p levels between ApoE4^{+/-} and ApoE4^{-/-} conditions are seen in neurons as well as in other brain cells such as astrocytes (FIG. 11C). The cell-type specific changes in miR-195-5p may contribute to different aspects of disease pathogenesis. These data show that reduction in neuronal miR-195-5p levels contributes to cognitive and synaptic dysfunction, while reduction in astrocytic miR-195-5p levels plays a role in defects in the secretory pathways leading to accelerated tau accumulation and spread.

Sex impact in miR-195-5p expression was also examined. In ROSMAP dataset, the differences in miR-195-5p levels between ApoE4^{+/-} and ApoE4^{-/-} carriers persist in female subjects (FIG. 1B), similarly to a previous report of sex-specific effects on brain PIP₂ homeostasis (Zhu L et al., Proc Natl Acad Sci USA 2015). Sex dimorphism is also noted in miR-195-5p expression with much lower levels in female subjects, particularly in ApoE4^{-/-} carriers (FIG. 7A). EFAD mice also exhibited sex dimorphic responses to miR-195-5p manipulations with improved cognitive function and reduced oligomer Aβ levels in male but not female EFAD mice (FIGS. 4C and 4E). Age-related changes in miR-195-5p expression and synj1 mRNA levels have also been noted in ApoE KI and EFAD mouse brains, with differential expression more prominent between ApoE4^{+/+} and ApoE4^{-/-} mice more prominent at 12 months of age compared to a younger age, whereas differences in miR-195-5p in EFAD mice are already evident at 4 months of age, suggesting that ApoE-genotype associated miR-195-5p changes can be exacerbated by aging and/or manifestations of AD pathologies.

While AD manifests as a multi-faceted disease process, targeting a specific miRNA to restore dysregulated networks and pathways at multiple levels could provide a promising avenue for future drug development. Therapeutic strategies directed at ApoE4 have been and are actively explored in several preclinical and clinical studies such as immunotherapies, antisense oligonucleotide treatments, gene editing, modulators of ApoE expression, as well as small molecules to enhance ApoE lipidation, to correct its structures, to compete receptor binding, and to inhibit ApoE-Aβ interaction (Cao J et al., Mol Neurodegener 2018, and Williams T et al., Mol Neurodegener 2020). The findings here show a therapeutic direction that modulates ApoE4 pathogenic function by a miRNA miR-195-5p through brain PIP₂ lipid signaling pathways with multiple beneficial effects besides impact on Aβ and tau pathology.

In summary, these studies show a mechanistic link between ApoE4 genotype-specific changes in brain miR-195-5p expression with AD-related phenotypes including brain phospholipid dysregulation, cognitive deficits, lysosomal defects, and tau pathologies. These studies also provide a therapeutic strategy for targeting at a specific miRNA miR-195-5p.

Abbreviations. AAV2: adeno-associated virus 2; AD: Alzheimer's Disease; ApoE: Apolipoprotein E; BH: Benjamini-Hochberg's; BSA: bovine serum albumin; CSF: cerebrospinal fluid; DEG: differentially expressed gene; FDR: false discovery rate; FGF2: fibroblast growth factor 2; FPKM: fragments per kilobase of transcript per million mapped reads; GSEA: gene set enrichment analysis; hiPSC: human inducible pluripotent stem cells; HPLC: high performance liquid chromatography iPSC: inducible pluripotent stem cells; KI: knock-in; MCI: mild cognitive impairment; miRNA: micro-RNA; NBTR: NIH Brain and Tissue Repository; NPC: neural progenitor cells; PI: phosphoinositol; PIP: phosphoinositol monophosphate; PIP₂: phosphoinositol biphosphate; PML: post-mortem interval; RAP: receptor associated protein; RIN: RNA integrity number; ROSMAP: Religious Orders Study and the Rush Memory and Aging Project; synj1: synaptojanin 1; TBI: traumatic brain injury; TMM: Trimmed Mean of M-values.

### Example 2: MicroRNA-195-5p is an Antiflammatory miRNA Regulating Microglial Function and can Alleviate Ischemia-Induced Microglial Dysfunction and Neuronal Injury

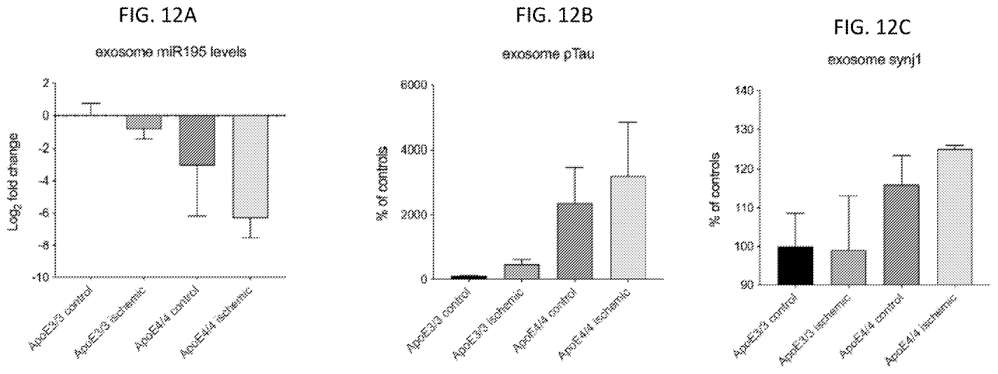

ApoE3 and ApoE4 iPSC-derived astrocytes were incubated for 3 days prior to treatment with sodium hydrosulfite (Na2S2O4) for 1 hour to induce ischemia followed by changes into serum-free culture media overnight for collection of exosomes. Then the exosomal content was characterized; and it was found that the miR-195-5p levels in exosomes derived from ischemic conditions are significantly lower than those in control conditions, with reciprocal increases in exosomal synj1 and pTau levels (FIG. 12).

As shown in FIG. 12, the amount of miR-195-5p in ApoE4 exosomes was less than that in ApoE3/3 exosomes and further reduced in ischemic conditions. In contrast, the levels of pTau and synj1 in exosome of ApoE4/4 (+/− ischemic conditions) are much higher than those in controls (presented as percentages of controls with 100% as ApoE3/3 control without ischemic conditions).

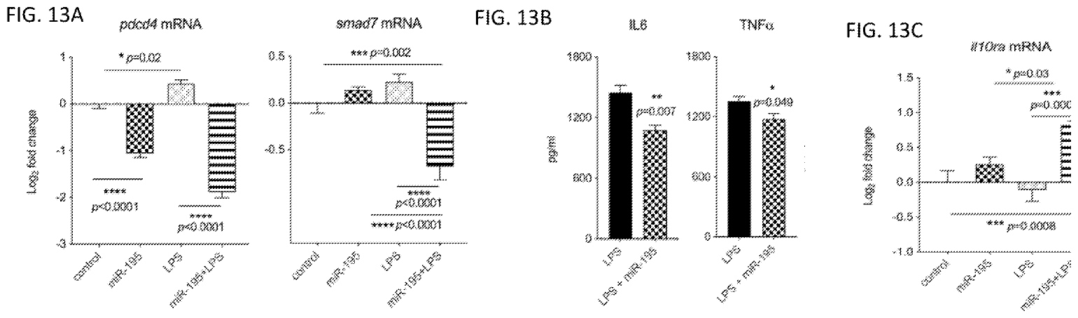

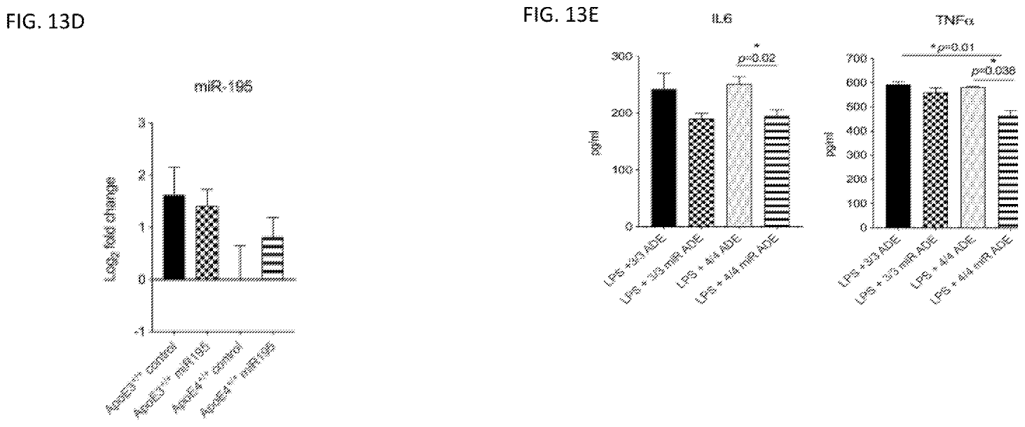

It was also found that over-expression of miR-195-5p in microglia inhibits LPS-induced increases in expression of pdcd4 and smad7, attenuates LPS-induced proinflammatory cytokine release and augments anti-inflammatory gene expression (FIG. 13). For example, LPS (at 0.2 μg/ml) treatment for overnight in BV2 cells leads to increased expression of pdcd4 and smad7 (FIG. 13A) and reduced expression of il10ra (FIG. 13C). However, over-expression of miR-195-5p dramatically reduces expression levels of pdcd4 and smad7 and increases il10a expression in the presence of LPS. Over-expression of miR-195-5p also attenuates LPS-induced pro-inflammatory cytokine release (IL-6 and TNFα; FIG. 13B). Interestingly, exosomes derived from APOE4/4 astrocytes (ADEs) contain less miR-195-5p compared to ADEs from APOE3/3 (FIG. 13D), and over-expression of miR-195-5p in iPSC-derived astrocytes leads to increased miR-195 levels in exosomes which can attenuate LSP-induced pro-inflammatory cytokine release (FIG. 13E). For these experiments, purified exosomes were derived from human APOE3/3 and 4/4 iPSC-derived astrocytes w/wo miR-195 over-expression detected by an exosome marker, ALIX. Fluorescence labeled exosomes were taken up by IBA1+ microglia cells. Together, these results support the role of miR-195 as an anti-inflammatory miRNA in regulating microglial function.

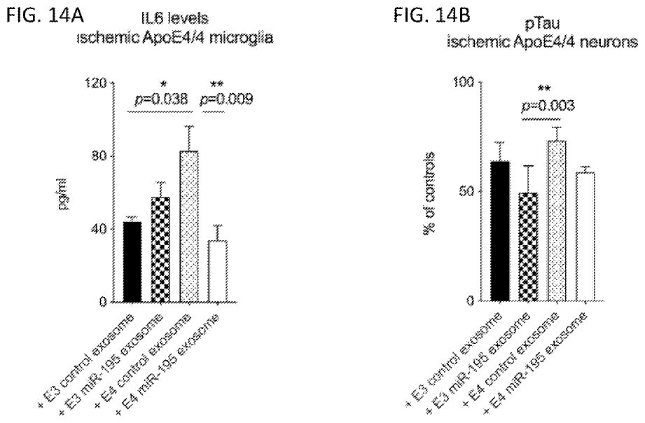

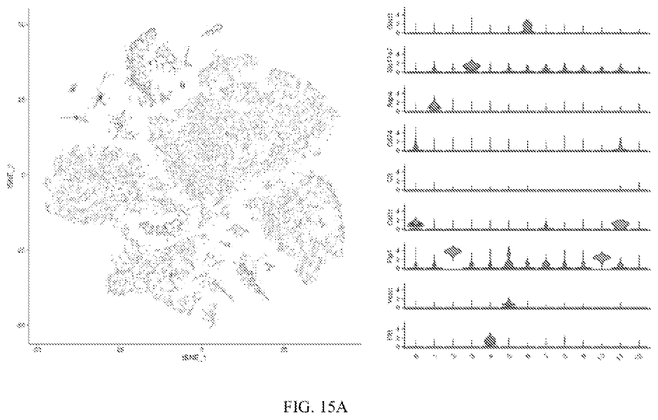

Treating ApoE4 microglia or neurons exposed to Na₂S₂O₄-induced ischemic conditions with exosomes derived from ApoE4 astrocytes overexpressing miR-195 reduced proinflammatory cytokine IL-6 release and decreased pTau production (FIG. 14). These data show a therapeutic role of exosomal miR-195 in alleviating ischemia induced microglial dysfunction and neuronal injury.

### Example 3: MicroRNA-195-5p Regulates Microglial Function and Neuroinflammation in Alzheimer's Disease

Changes in microglia-specific gene profiles with miR-195-5p over-expression in E4FAD mouse brain. scRNA-seq analysis of EFAD mouse brains with miR-195-5p over-expression was performed (FIG. 15; N=4 pooled mice/condition). Single cell suspensions were prepared and processed with the 10× Genomics Chromium platform. After quality control (QC), the gene-level UMIs data were normalized by a regularized negative binomial regression analysis (Hafemeister, C. & Satija, R. Genome Biology 20, 296, (2019)). Genes were selected that were detected in more than one cell and cells with sequencing reads in 200˜4,000 genes and mitochondrial read rate of less than 50%. This resulted in a dataset of total 26,332 cells and 18,834 genes for further analysis. Next, linear dimensional reduction were first performed using principal component analysis (PCA). The significant principal components as determined by a JackStraw permutation procedure were selected for cell clustering using Seurat's graph-based clustering approach (Butler, A., et al. Nature Biotechnology 36, 411, (2018)). The normalized dataset was projected onto a 2D space using t-distributed Stochastic Neighbor Embedding (t-SNE; FIGS. 15A and 15C) (van der Maaten, L. & Hinton, G. Machine Learning 87, 33-55 (2012)) or Uniform Manifold Approximation and Projection for Dimension Reduction (UMAP) (McInnes, L., Healy, J. & Melville, J. arXiv 1802.03426 (2018)). To identify and remove doublet artifacts in the scRNA-seq data, DoubletFinder (McGinnis, C. S., et al. Cell systems 8, 329-337 (2019)) was used, an R package implemented to interface with Seurat. After removing predicted doublets from the clusters, the cluster marker genes were observed and the expression patterns of known gene markers were analyzed to annotate clusters into major cell-types: excitatory neurons (Ex; marked by NRGN), inhibitory neurons (In; GAD1), astrocytes (Ast; AQP4, GFAP), oligodendrocytes (Oli; PLP1, MBP), microglia (Mic; CSF1R, CD74), oligodendrocyte progenitor cells (Opc; VCAN), and endothelial cells (End; FLT1). To identify cluster-specific signatures or differentially expressed genes (DEGs) between sample groups, non-parametric tests such as Wilcoxon rank sum test were employed. By employing a deep neural network-based single-cell clustering approach, DESC (Li, X. et al. bioRxiv, 530378 (2019)), twelve cell clusters were identified with annotation to known major brain cell types including microglia (C0), astrocytes (C1), neurons (C3) and oligodendrocytes (C2) (FIG. 15A). Top GO pathways enrichment studies of cluster-specific DEGs suggest that miR-195-5p over-expression up-regulates genes involved in mitochondrial and synaptic function within microglial cluster (C0) (FIG. 15B). Sub clustering of the microglial cluster (C0) identified three major subsets (FIG. 15C) with different gene signatures in each microglia sub-cluster (FIG. 15D). For example, sub-cluster Mic.C0 was enriched for genes involved in regulation of cell death and response to cytokine, sub-cluster Mic.C1 was enriched for MHC class II protein complex, myeloid leukocyte activation and vacuole, while sub-cluster Mic.C2 was enriched for NADH dehydrogenase activity. Studies of top GO pathways enriched with sub-cluster DEGs suggest that miR-195-5p down-regulates innate immune system and effector responses in Mic.C0 and Mic.C2 microglial sub-clusters, as well as translation and ribosome activities in Mic.C1 sub-cluster, and up-regulates genes involved in oxidative phosphorylation and ATP metabolic processes in three sub-clusters. These data show a role of miR-195-5p in the regulation of microglial function and neuro-inflammation in AD.

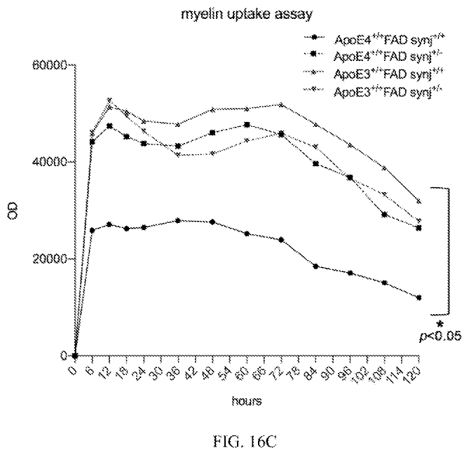

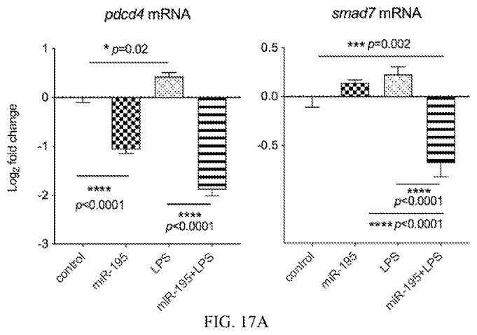

APOE4⁺ microglia with reduced miR-195 levels and increased Synj1 expression manifest with impaired phagocytic activities and lysosomal enlargement that are rescued by Synj1 reduction. miR-195 levels in APOE4⁺ neurons and astrocytes are reduced with increased synj1 expression compared to that in APOE3⁺ cells (Cao, J. et al. Molecular psychiatry, (2020)). Data further suggests that miR195 levels are lower with higher synj1 protein expression levels in cultured APOE4⁺ microglia compared to those in APOE3⁺ microglia (FIGS. 16A and B). Using PHrodo-conjugated myelin to determine microglial phagocytic activities, it was found that APOE4^{+/+} synj1^{+/+} microglia manifests with reduced amounts of myelin uptake, and that these phenotypes are most prominent at the first 6 hours of incubation and remain constant throughout 72 hours of studies when compared to APOE3^{+/+}synj1^{+/+} microglia, suggesting impaired phagocytic activities in these microglia (FIG. 16C). In addition, the slope of degradation of myelin taken inside of microglia was slower in APOE4^{+/+}synj1^{+/+} microglia than that of APOE3^{+/+}syn1^{+/+} microglia. Furthermore, lysosomal enlargement was observed in cultured APOE4^{+/+}synj1^{+/+} microglia, similar to observations in human iPSC-derived AD APOE4^{+/+} neuron and astrocyte co-culture (FIG. 16B and Cao, J. et al. Molecular psychiatry, (2020). These phenotypes can be rescued with synj1 haploinsufficiency (FIG. 16C black dashed curve for APOE4^{+/+}synj1^{+/-} microglia), with no significant differences seen between microglial culture of APOE3^{+/+}synj1^{+/+} and APOE3^{+/+}synj1^{+/-} genotypes. These findings suggest a regulatory role of miR-195/synj1 in APOE4-induced microglial dysfunction.

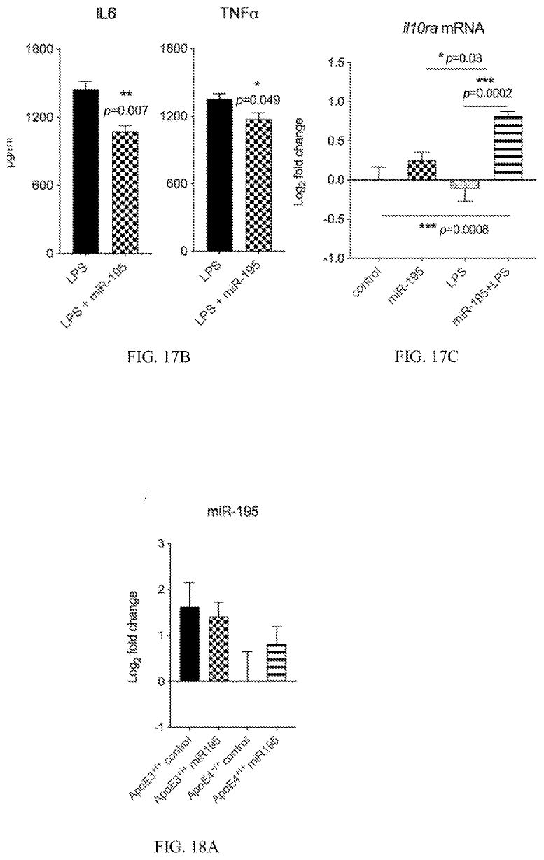

Over-expression of miR-195-5p in microglia inhibits LPS-induced proinflammatory responses and augments anti-inflammatory responses. The results show that over-expression of miR-195-5p in microglia inhibits LPS-induced increases in expression of inflammatory genes pdcd4 and smad7, attenuates LPS-induced proinflammatory cytokine release and augments anti-inflammatory gene expression. The results also show that LPS (at 0.2 g/ml) treatment for overnight in BV2 cells leads to increased expression of pdcd4 and smad7 (FIG. 17A) and reduced expression of il10ra (FIG. 17C). However, over-expression of miR-195-5p reduces expression levels of pdcd4 and smad7 and increases il10a expression in the presence of LPS. Over-expression of miR-195-5p also attenuates LPS-induced pro-inflammatory cytokine release (IL-6 and TNFα; FIG. 17B).

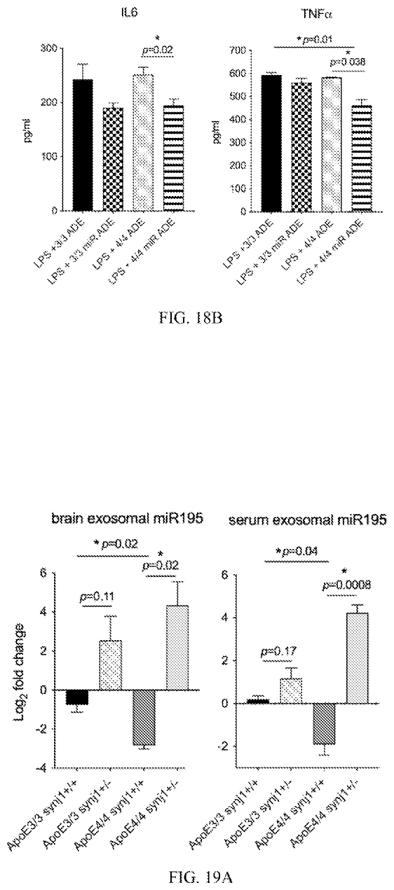

Moreover, exosomes derived from APOE4/4 astrocytes (ADEs) contained much lower miR-195-5p levels than those in ADEs from APOE3/3 and over-expression of miR-195-5p increased exosomal miR-195-5p levels (FIG. 18A), which can attenuate LSP-induced pro-inflammatory cytokine release (FIG. 18B). A picture of uptake of fluorescence-labeled exosomes into cultured microglia shows that exosomal miR-195 uptake into microglia modulates inflammatory responses. Purified exosomes derived from human APOE3/3 and 4/4 iPSC-derived astrocytes with and without miR-195 over-expression were detected by an exosome marker, ALIX. Fluorescence labeled exosomes were taken up by IBA1l mouse E3FAD or E4FAD microglia. Together, these results support the role of miR-195-5p as an anti-inflammatory miRNA in modulating microglial function.

### Example 4: A Role of Exosomal miR-195-5p as a Target Engagement Biomarker for Brain ApoE-Synj1-PIP₂ Pathway

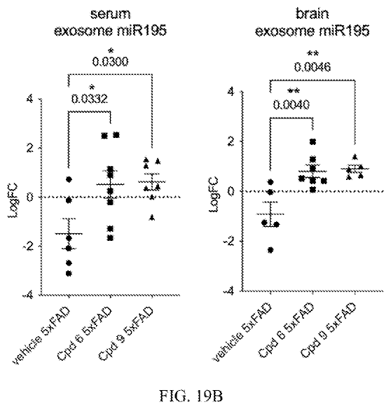

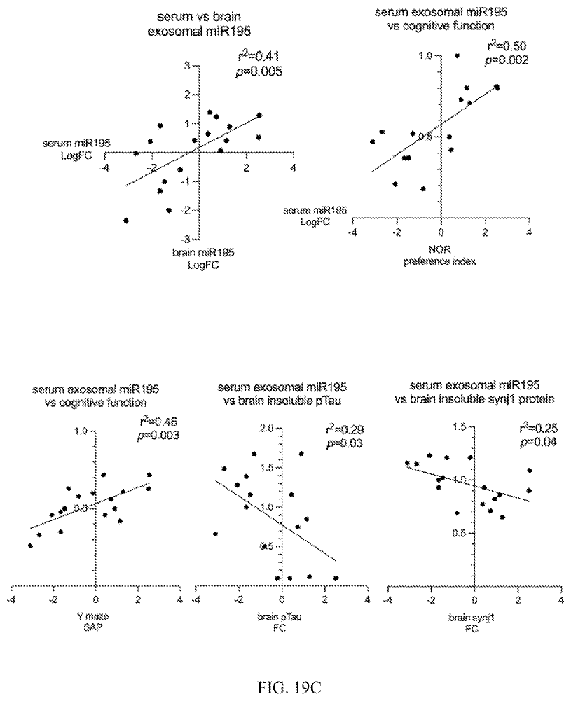

As disclosed herein, a study was performed to examine exosomal fractions of brain and serum samples of synj1 haploinsufficiency mice (ApoE3 synj1^{+/-} and ApoE4 synj1^{+/-}) as well as of mice treated with synj1-lowering agents (SynaptoCpd #9 and Cpd #6). Exosomes were purified using sucrose gradient fractionation methods and the purity of exosome fractions were evaluated by western blot analysis of exosomal protein markers such as ALIX, and uptake studies of fluorescence tagged exosomes by human iPSC-derived astrocyte cultures. The results show that brain and serum exosomal miR-195-5p levels were lower in ApoE4 synj1^{+/+} mice when compared ApoE3 synj1^{+/+} mice. In synj1 haploinsufficiency mice, there were significantly increased exosomal miR-195-5p in brain and serum samples of ApoE4 synj1^{+/-} mice (FIG. 19A). Moreover, it was found that serum and brain exosomal miR-195-5p levels were much higher in Cpd #6 or Cpd #9-treated mice when compared to controls (FIG. 19B). In addition, serum exosomal miR-195-5p levels were positively correlated with brain exosomal miR-195-5p levels and cognitive performance (NOR preference index and Y maze SAP scores), and reversely correlated with brain insoluble pTau and synj1 protein levels in drug-treated mouse cohorts (FIG. 19C).

What is claimed is:

1. A method of treating cognitive impairment in a subject, the method comprising:
administering to the subject a therapeutically effective amount of a composition comprising miR-195-5p or a fragment or variant thereof.

2. The method of claim 1, further comprising determining the expression level of a miR-195-5p in a sample obtained from the subject before the administration of the composition comprising miR-195-5p, wherein the expression level of miR-195-5p is lower when compared to a reference sample.

3. The method of claim 2, wherein the reference sample is obtained from a subject that does not have or has not been diagnosed as having cognitive impairment.

4. A method of treating cognitive impairment in a subject, the method comprising:
administering a composition comprising miR-195-5p to the subject, wherein the subject has been diagnosed with a cognitive impairment by:
i) determining, in a sample obtained from the subject, the expression level of a miR-195-5p, and
ii) comparing the expression level of the miR-195-5p in the sample obtained from the subject with the expression level of the miR-195-5p in a reference sample,
wherein a lower expression level of the miR-195-5p in the sample obtained from the subject indicates a cognitive impairment in the subject.

5. The method of claim 4, wherein the reference sample is obtained from a subject that does not have or has not been diagnosed as having said cognitive impairment.

6. A method of ameliorating one or more symptoms of cognitive impairment in a subject, the method comprising administering to the subject a therapeutically effective amount of a composition comprising miR-195-5p or a fragment or variant thereof.

7. A method of reducing synaptojanin 1 (synj1) activity or expression in a subject, the method comprising administering to the subject a therapeutically effective amount of a composition comprising miR-195-5p or a fragment or variant thereof.

8. A method of inhibiting synaptojanin 1 (synj1) activity or expression in a subject, the method comprising administering to the subject a therapeutically effective amount of a composition comprising miR-195-5p or a fragment or variant thereof.

9. A method of increasing amyloid β-protein (Aβ) clearance in a subject, the method comprising administering to the subject a therapeutically effective amount of a composition comprising miR-195-5p or a fragment or variant thereof.

10. A method of reducing traumatic brain injury (TBI)-induced elevation in tau hyper-phosphorylation in a subject, the method comprising administering to the subject a therapeutically effective amount of a composition comprising miR-195-5p or a fragment or variant thereof.

11. A method of reducing amyloid plaque burden in a subject, the method comprising administering to the subject a therapeutically effective amount of a composition comprising miR-195-5p or a fragment or variant thereof.

12. A method of reducing tau hyper-phosphorylation in a subject, the method comprising administering to the subject a therapeutically effective amount of a composition comprising miR-195-5p or a fragment or variant thereof.

13. A method of reducing IL-6 or TNFα release in a subject, the method comprising administering to the subject a therapeutically effective amount of a composition comprising miR-195-5p or a fragment or variant thereof.

14. A method of decreasing phosphorylated tau production in a subject, the method comprising administering to the subject a therapeutically effective amount of a composition comprising miR-195-5p or a fragment or variant thereof.

15. A method of treating or alleviating ischemia induced microglial dysfunction and neuronal injury in a subject, the method comprising administering to the subject a therapeutically effective amount of a composition comprising miR-195-5p or a fragment or variant thereof.

16. A method of rescuing Alzheimer's disease-related lysosomal defects in a subject, the method comprising administering to the subject a therapeutically effective amount of a composition comprising miR-195-5p or a fragment or variant thereof.

17. The method of claims 1-6, wherein the cognitive impairment is Alzheimer's disease, mild cognitive impairment, Lewy body dementia (LBD), frontotemporal dementia (FTD), vascular dementia, mixed dementia, or Down Syndrome.

18. The method of any of the preceding claims, wherein the subject is identified in need of treatment before the administering step.

19. The method of any of the preceding claims, wherein the subject is a human.

20. The method of any of the preceding claims, wherein the miR-195-5p or the fragment or variant thereof is administered systemically.

21. The method of claim 20, wherein miR-195-5p or the fragment or variant thereof is located in a vector.

22. The method of claim 21, wherein the vector is a plasmid, cosmid, phagemid or a viral vector.

23. The method of claim 21, wherein the vector further comprises a lipid, lipid emulsion, liposome, nanoparticle or exosomes.

24. The method of claim 20, wherein miR-195-5p or the fragment or variant is comprised in a lipid, lipid emulsion, liposome, nanoparticle or exosome.

25. The method of claim 22, wherein the viral vector is an adenovirus, an adeno-associated virus, a lentivirus or a herpes simplex virus.

26. The method of any of the preceding claims, wherein the miR-195-5p is hsa-miR-195-5p comprising the nucleotide sequence set forth in SEQ ID NO: 1.

27. The method of any of the preceding claims, further comprising administering a therapeutically effective amount of a composition comprising a compound selected from the group consisting of:

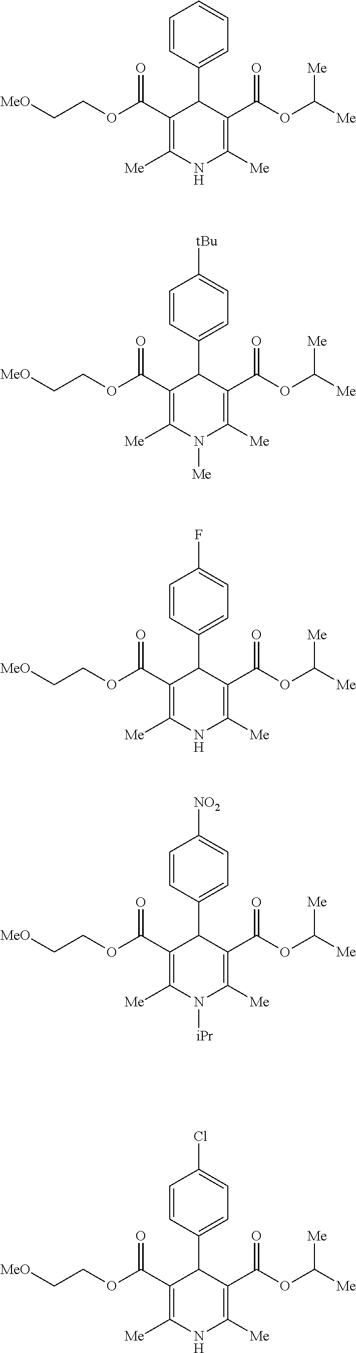 [SMILES: COCCOC(=O)C1=C(C)NC(C)=C(C(=O)OC(C)C)C1c1ccccc1 ; COCCOC(=O)C1=C(C)N(C)C(C)=C(C(=O)OC(C)C)C1c1ccc(C(C)(C)C)cc1 ; COCCOC(=O)C1=C(C)NC(C)=C(C(=O)OC(C)C)C1c1ccc(F)cc1 ; COCCOC(=O)C1=C(C)N(C(C)C)C(C)=C(C(=O)OC(C)C)C1c1ccc([N+](=O)[O-])cc1 ; COCCOC(=O)C1=C(C)NC(C)=C(C(=O)OC(C)C)C1c1ccc(Cl)cc1]

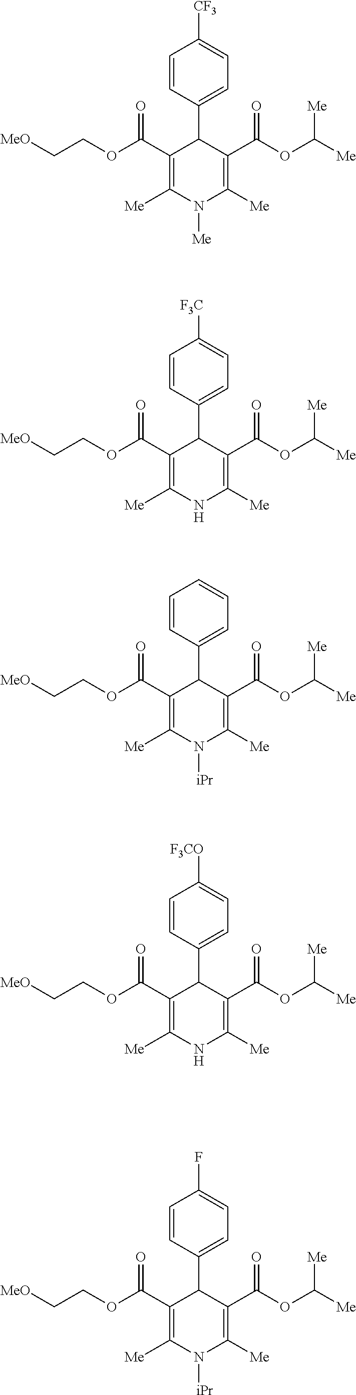 [SMILES: COCCOC(=O)C1=C(C)N(C)C(C)=C(C(=O)OC(C)C)C1c1ccc(C(F)(F)F)cc1 ; COCCOC(=O)C1=C(C)NC(C)=C(C(=O)OC(C)C)C1c1ccc(C(F)(F)F)cc1 ; COCCOC(=O)C1=C(C)N(C(C)C)C(C)=C(C(=O)OC(C)C)C1c1ccccc1 ; COCCOC(=O)C1=C(C)NC(C)=C(C(=O)OC(C)C)C1c1ccc(OC(F)(F)F)cc1 ; COCCOC(=O)C1=C(C)N(C(C)C)C(C)=C(C(=O)OC(C)C)C1c1ccc(F)cc1]

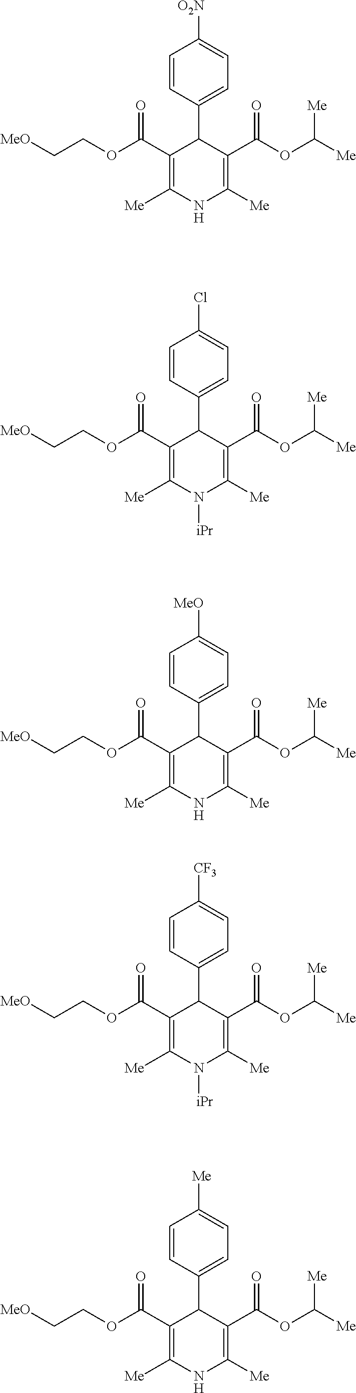 [SMILES: COCCOC(=O)C1=C(C)NC(C)=C(C(=O)OC(C)C)C1c1ccc([N+](=O)[O-])cc1 ; COCCOC(=O)C1=C(C)N(C(C)C)C(C)=C(C(=O)OC(C)C)C1c1ccc(Cl)cc1 ; COCCOC(=O)C1=C(C)NC(C)=C(C(=O)OC(C)C)C1c1ccc(OC)cc1 ; COCCOC(=O)C1=C(C)N(C(C)C)C(C)=C(C(=O)OC(C)C)C1c1ccc(C(F)(F)F)cc1 ; COCCOC(=O)C1=C(C)NC(C)=C(C(=O)OC(C)C)C1c1ccc(C)cc1]

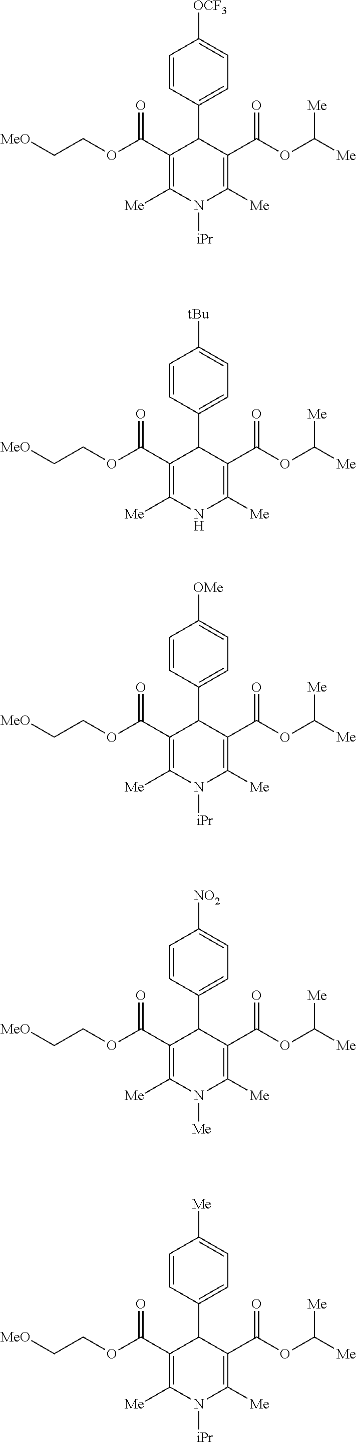 [SMILES: COCCOC(=O)C1=C(C)N(C(C)C)C(C)=C(C(=O)OC(C)C)C1c1ccc(OC(F)(F)F)cc1 ; COCCOC(=O)C1=C(C)NC(C)=C(C(=O)OC(C)C)C1c1ccc(C(C)(C)C)cc1 ; COCCOC(=O)C1=C(C)N(C(C)C)C(C)=C(C(=O)OC(C)C)C1c1ccc(OC)cc1 ; COCCOC(=O)C1=C(C)N(C)C(C)=C(C(=O)OC(C)C)C1c1ccc([N+](=O)[O-])cc1 ; COCCOC(=O)C1=C(C)N(C(C)C)C(C)=C(C(=O)OC(C)C)C1c1ccc(C)cc1]

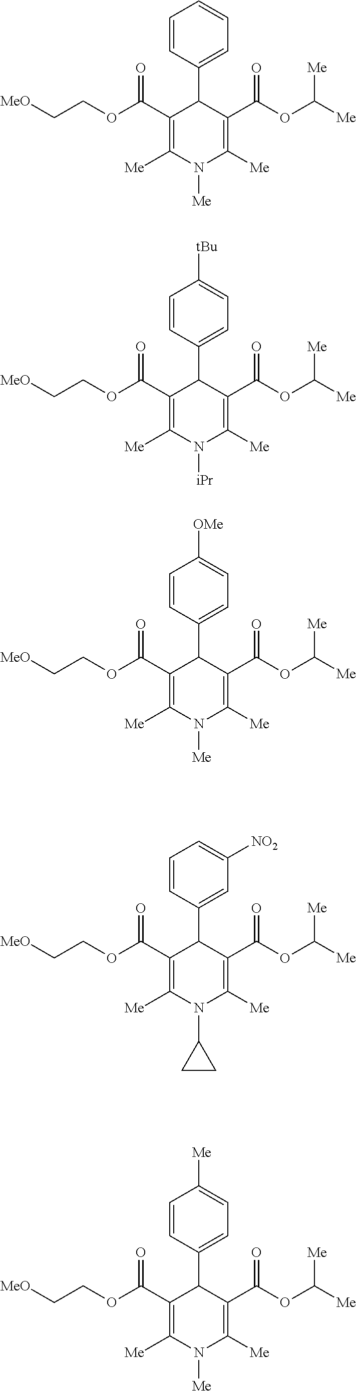

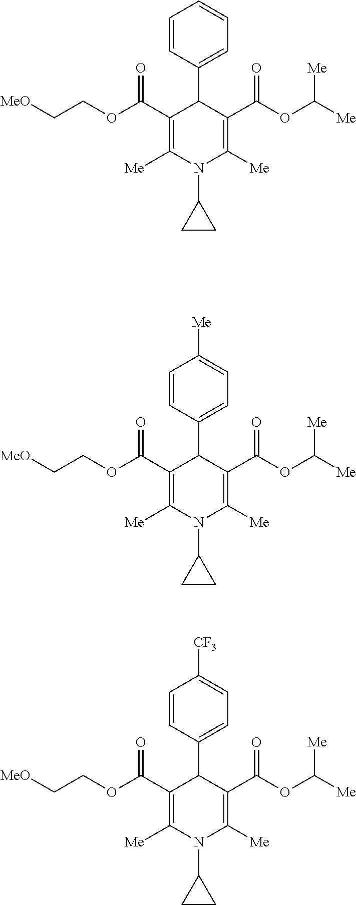 [SMILES: COCCOC(=O)C1=C(C)N(C2CC2)C(C)=C(C(=O)OC(C)C)C1c1ccccc1 ; COCCOC(=O)C1=C(C)N(C2CC2)C(C)=C(C(=O)OC(C)C)C1c1ccc(C)cc1 ; COCCOC(=O)C1=C(C)N(C2CC2)C(C)=C(C(=O)OC(C)C)C1c1ccc(C(F)(F)F)cc1]

28. A method comprising:
(a) obtaining or having obtained a plasma, serum or cerebrospinal fluid sample from a subject;
(b) measuring the expression level of miR-195-5p in the plasma, serum or cerebrospinal fluid sample;
(c) identifying the subject as being in need for treatment with a composition comprising miR-195-5p when the level of miR-195-5p is lower than a level of miR-195-5p in a control sample; and
(d) administering a composition comprising miR-195-5p or a fragment or variant thereof to the subject identified as in need of treatment.

29. The method of claim 28, wherein the subject has a cognitive impairment.

30. The method of claim 29, wherein the cognitive impairment is Alzheimer's disease, mild cognitive impairment, Lewy body dementia (LBD), frontotemporal dementia (FTD), vascular dementia, cerebrovascular disease, ischemic, mixed dementia, or Down Syndrome.

31. The method of claim 28, wherein the composition comprising miR-195-5p is administered systemically, intranasally or intrathecally.

32. The method of claim 31, wherein the miR-195-5p is located in a vector.

33. The method of claim 32, wherein the vector is a plasmid, cosmid, phagemid or viral vector.

34. The method of claim 32, wherein the vector comprises a lipid, lipid emulsion, liposome, nanoparticle or exosomes.

35. The method of claim 28, wherein the miR-195-5p is comprised in a lipid, lipid emulsion, liposome, nanoparticle or exosome.

36. The method of claim 33, wherein the viral vector is an adenovirus, an adeno-associated virus, a lentivirus or a herpes simplex virus.

37. The method of claim 32, wherein the vector comprises a lipid, lipid emulsion, liposome, nanoparticle or exosomes.

38. The method of claim 28, further comprising administering a therapeutically effective amount of a composition comprising a compound selected from the group consisting of:

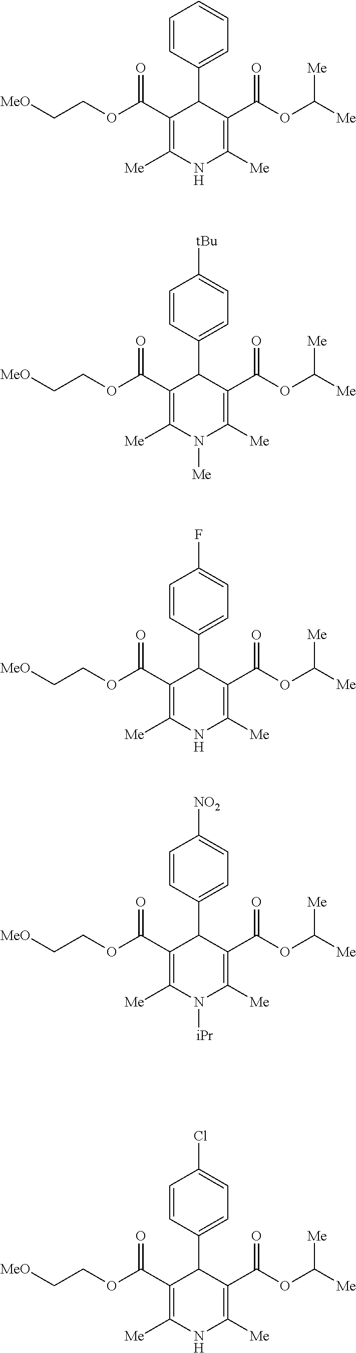 [SMILES: COCCOC(=O)C1=C(C)NC(C)=C(C(=O)OC(C)C)C1c1ccccc1 ; COCCOC(=O)C1=C(C)N(C)C(C)=C(C(=O)OC(C)C)C1c1ccc(C(C)(C)C)cc1 ; COCCOC(=O)C1=C(C)NC(C)=C(C(=O)OC(C)C)C1c1ccc(F)cc1 ; COCCOC(=O)C1=C(C)N(C(C)C)C(C)=C(C(=O)OC(C)C)C1c1ccc([N+](=O)[O-])cc1 ; COCCOC(=O)C1=C(C)NC(C)=C(C(=O)OC(C)C)C1c1ccc(Cl)cc1]

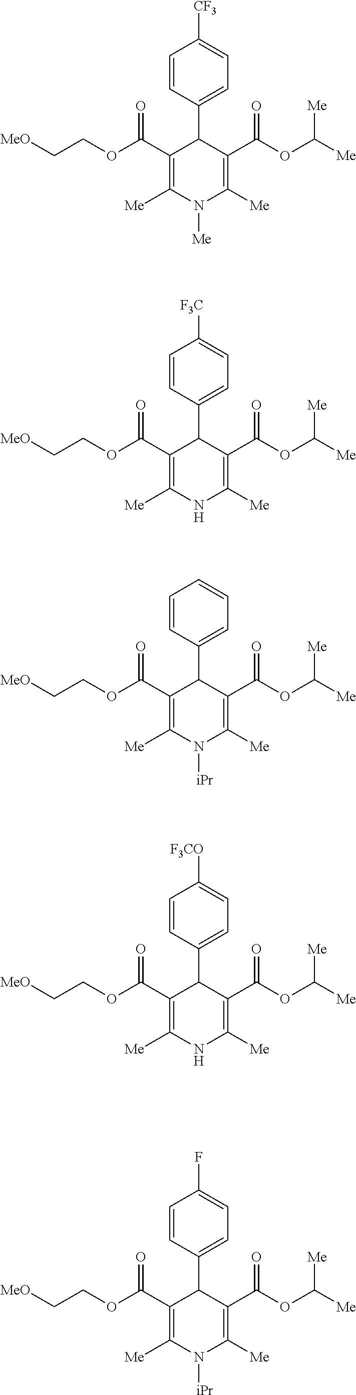 [SMILES: COCCOC(=O)C1=C(C)N(C)C(C)=C(C(=O)OC(C)C)C1c1ccc(C(F)(F)F)cc1 ; COCCOC(=O)C1=C(C)NC(C)=C(C(=O)OC(C)C)C1c1ccc(C(F)(F)F)cc1 ; COCCOC(=O)C1=C(C)N(C(C)C)C(C)=C(C(=O)OC(C)C)C1c1ccccc1 ; COCCOC(=O)C1=C(C)NC(C)=C(C(=O)OC(C)C)C1c1ccc(OC(F)(F)F)cc1 ; COCCOC(=O)C1=C(C)N(C(C)C)C(C)=C(C(=O)OC(C)C)C1c1ccc(F)cc1]

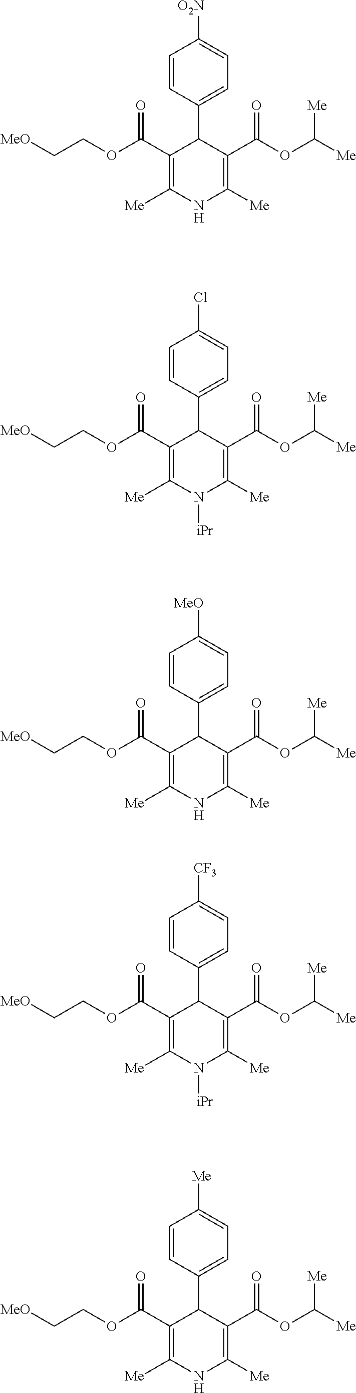 [SMILES: COCCOC(=O)C1=C(C)NC(C)=C(C(=O)OC(C)C)C1c1ccc([N+](=O)[O-])cc1 ; COCCOC(=O)C1=C(C)N(C(C)C)C(C)=C(C(=O)OC(C)C)C1c1ccc(Cl)cc1 ; COCCOC(=O)C1=C(C)NC(C)=C(C(=O)OC(C)C)C1c1ccc(OC)cc1 ; COCCOC(=O)C1=C(C)N(C(C)C)C(C)=C(C(=O)OC(C)C)C1c1ccc(C(F)(F)F)cc1 ; COCCOC(=O)C1=C(C)NC(C)=C(C(=O)OC(C)C)C1c1ccc(C)cc1]

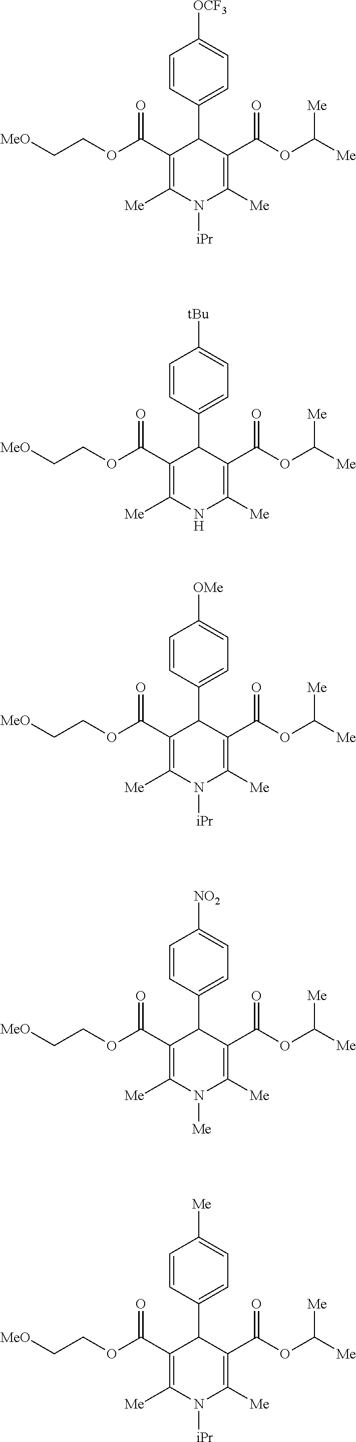 [SMILES: COCCOC(=O)C1=C(C)N(C(C)C)C(C)=C(C(=O)OC(C)C)C1c1ccc(OC(F)(F)F)cc1 ; COCCOC(=O)C1=C(C)NC(C)=C(C(=O)OC(C)C)C1c1ccc(C(C)(C)C)cc1 ; COCCOC(=O)C1=C(C)N(C(C)C)C(C)=C(C(=O)OC(C)C)C1c1ccc(OC)cc1 ; COCCOC(=O)C1=C(C)N(C)C(C)=C(C(=O)OC(C)C)C1c1ccc([N+](=O)[O-])cc1 ; COCCOC(=O)C1=C(C)N(C(C)C)C(C)=C(C(=O)OC(C)C)C1c1ccc(C)cc1]

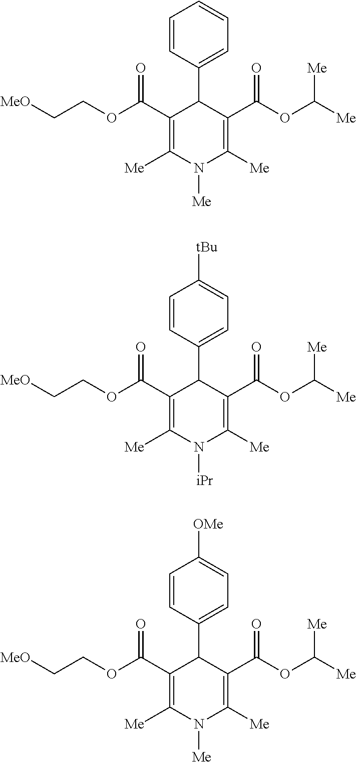

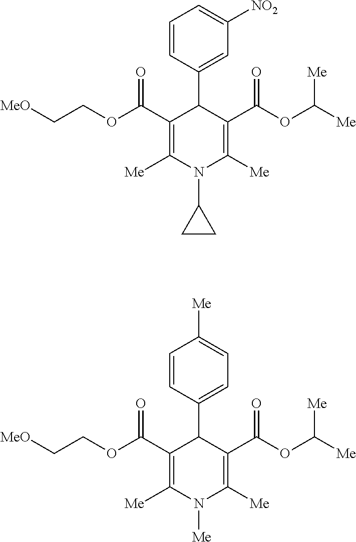 [SMILES: COCCOC(=O)C1=C(C)N(C2CC2)C(C)=C(C(=O)OC(C)C)C1c1cccc([N+](=O)[O-])c1 ; COCCOC(=O)C1=C(C)N(C)C(C)=C(C(=O)OC(C)C)C1c1ccc(C)cc1]

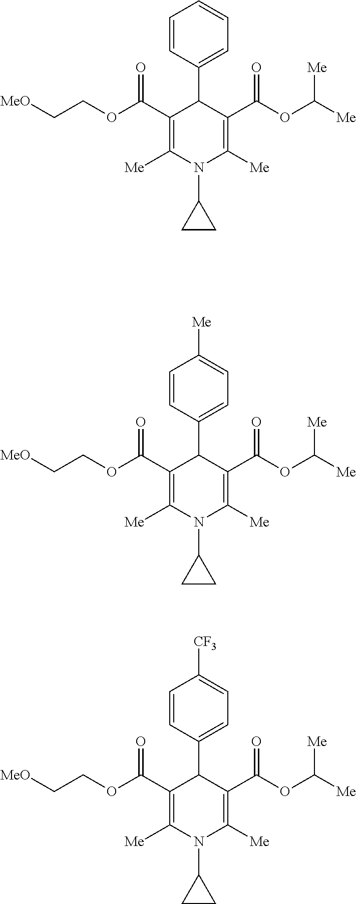 [SMILES: COCCOC(=O)C1=C(C)N(C2CC2)C(C)=C(C(=O)OC(C)C)C1c1ccccc1 ; COCCOC(=O)C1=C(C)N(C2CC2)C(C)=C(C(=O)OC(C)C)C1c1ccc(C)cc1 ; COCCOC(=O)C1=C(C)N(C2CC2)C(C)=C(C(=O)OC(C)C)C1c1ccc(C(F)(F)F)cc1]

39. A method of diagnosing a subject with a cognitive impairment the method comprising:
a) measuring the expression level of miR-195-5p in a sample obtained from the subject;
b) determining the subject has said cognitive impairment if the expression level of miR-195-5p is lower than the expression level of miR-195-5p of a reference sample, wherein the corresponding reference value is the average value of the expression level of miR-195-5p in healthy subjects; and
c) treating the subject for said cognitive impairment.

40. The method of claim 39, wherein the expression level of miR-195-5p is determined by quantitative PCR.

41. The method of claim 40, wherein the step of treating the subject for said cognitive impairment comprises administering to the subject a therapeutically effective amount of a composition comprising miR-195-5p.

42. A method of determining whether a subject has a cognitive impairment, the method comprising:
a) detecting the expression level of miR-195-5p in a sample obtained from the subject;
b) comparing the expression level of miR-195-5p in the sample from the subject to the expression level of miR-195-5p from a reference sample; and
c) determining the subject does not have a cognitive impairment when the expression level of miR-195-5p in the subject's sample is the same or higher than the expression level of miR-195-5p from a reference sample or determining the subject does have a cognitive impairment when the expression level of miR-195-5p in the subject's sample is lower than the level of miR-195-5p from the reference sample.

43. The method of claim 42, further comprising administering to the subject diagnosed with said cognitive impairment a therapeutically effective amount of a composition comprising miR-195-5p.

44. The method of any of the claims above, wherein the sample is serum, cerebrospinal fluid or plasma.


SEQUENCE LISTING (5 sequences)

SEQ ID NO: 1 - RNA, 21 nt, Artificial Sequence
  Synthetic construct
uagcagcacagaaauauuggc

SEQ ID NO: 2 - RNA, 22 nt, Artificial Sequence
  Synthetic construct
ccaauauuggcugugcugcucc

SEQ ID NO: 3 - RNA, 7 nt, Artificial Sequence
  Synthetic construct
agcagca

SEQ ID NO: 4 - RNA, 87 nt, Artificial Sequence
  Synthetic construct
agcuucccuggcucuagcagcacagaaauauuggcacagggaagcgagucugccaauauuggcugugcugcuccaggcaggguggug

SEQ ID NO: 5 - RNA, 21 nt, Artificial Sequence
  Synthetic construct
uagcagcacagaaauauuggc